(19) United States
(12) Patent Application Publication
(10) Pub. No.: US 2026/0308538 A1
(43) Pub. Date: Oct. 8, 2026

(54) SYNTHETIC MMAB GENES AND AAV VECTORS TO TREAT COBALAMIN B DEFICIENCY

(71) Applicant: The United States of America, as represented by the Secretary, Department of Health and Human Servi, Bethesda, MD (US)

(72) Inventors: Charles P. Venditti, Potomac, MD (US); Eun-Young Choi, Bethesda, MD (US)

(73) Assignee: The United States of America, as represented by the Secretary, Department of Health and Human Servi, Bethesda, MD (US)

(21) Appl. No.: 19/478,200

(22) PCT Filed: Apr. 5, 2024
(86) PCT No.: PCT/US2024/023432

§ 371 Date: Oct. 23, 2025

Related U.S. Application Data
(60) Provisional application No. 63/499,013, filed on Apr. 28, 2023.

(51) Int. Cl. C12N 9/10; A61K 38/45; A61K 48/00; A61P 3/00; C12N 15/86

(52) U.S. Cl. CPC C12N 9/1085; A61K 38/45; A61K 48/005; A61P 3/00; C12N 15/86; C12Y 205/01017; C12N 2750/14143

(57) ABSTRACT

Synthetic polynucleotides encoding human MMAB (Metabolism Of Cobalamin Associated B) enzyme (synMMAB) and exhibiting augmented expression in cell culture and/or in a mammal are described herein. Related recombinant expression vectors, host cells, populations of cells, and pharmaceutical compositions relating to the synMMAB polynucleotides are also described. Methods of treating a disease or condition mediated by MMAB enzyme are also described.

### CROSS-REFERENCE TO RELATED APPLICATIONS

This patent application claims the benefit of U.S. Provisional Patent Application No. 63/499,013, filed Apr. 28, 2023, which is incorporated by reference.

### STATEMENT REGARDING FEDERALLY SPONSORED RESEARCH OR DEVELOPMENT

This invention was made with Government support under project number 1ZIAHG200318-19 by the National Institutes of Health, National Human Genome Research Institute. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 96,899 Byte XML file named “770632.XML,” dated Apr. 5, 2024.

### BACKGROUND OF THE INVENTION

Isolated methylmalonic acidemia (MMA) comprises a relatively common and heterogeneous group of inborn errors of metabolism. There are four major distinct genetic etiologies of isolated MMA. Mutations in the methylmalonyl-CoA mutase (MMUT) apoenzyme, the mitochondrial 5′-deoxyadenosylco(I)balamin transferase (MMAB) (Metabolism Of Cobalamin Associated B), or in the MMAA (Metabolism Of Cobalamin Associated A) enzyme define the mut, cb1B, and cb1A complementation groups, respectively. Cells from patients with absent MMUT activity are designated mut°; cells with detectable but abnormal activity are designated mut. Children with MMAA mutations have a greater chance of responding clinically to supplemental vitamin B₁₂, and usually are not as severely affected as children with mut forms of MMA. Most of those with MAB mutations (cb1B class MMA) clinically resemble patients with mut° MMA for the reason that they have completely absent MMUT activity because they cannot synthesize 5′-deoxyadenosylcobalamin, the cofactor for the hMUT enzyme. Patients with cb1D-variant 2 MMA, an ultra-rare inborn error of metabolism (IEM) with only several patients in the world recognized to date, resemble mut° and non-B12 responsive cb1B patients in terms of biochemical and clinical severity.

Most affected individuals with vitamin B12 non-responsive isolated MMA display severe multisystemic disease characterized by metabolic instability, chronic renal disease, and neurological complications. The treatment entails adherence to a low protein diet, carnitine supplementation and vigilant clinical monitoring. However, despite meticulous medical management, patients with MMUT and MMAB deficiency suffer from substantial mortality and morbidity related to the disease. Most commonly, premature death occurs in the setting of an acute metabolic crisis and has led to the use of elective liver or liver/kidney transplantation as a treatment some patients. While these experimental surgical procedures do not completely cure the biochemical phenotype, metabolic stability is restored after liver transplantation and the propensity for acute decompensation is eliminated. However, the patients must adhere to a life-long regimen of anti-rejection and immune suppression medicines and suffer the sequelae experienced by all transplant recipients, including the risk for graft failure, rejection, and have an increased incidence of malignancy. There are currently no gene therapies that have been developed for this severe pediatric metabolic disorder. Accordingly, there exists a need for improved compositions and methods for treating diseases and conditions mediated by MMAB enzyme.

### BRIEF SUMMARY OF THE INVENTION

An aspect of the invention provides a synthetic MMAB (Metabolism Of Cobalamin Associated B) polynucleotide (synMMAB) selected from the group consisting of: (a) a polynucleotide comprising the nucleic acid sequence of any one of SEQ ID NOs: 1-6; (b) a polynucleotide having a nucleic acid sequence with at least about 80% identity to the nucleic acid sequence of any one of SEQ ID NOs: 1-6 and encoding a polypeptide having an amino acid sequence at least about 90% identical to SEQ ID NO: 21 and having equivalent or increased expression in a mammalian host cell relative to expression of SEQ ID NO: 7 in the same host cell, wherein the polynucleotide does not have the nucleic acid sequence of SEQ ID NO: 7; and (c) a polynucleotide having a nucleic acid sequence with at least about 80% identity to the nucleic acid sequence of nucleotides 97-753 of any one of SEQ ID NOs: 1-6 and encoding a polypeptide having an amino acid sequence at least about 90% identical to SEQ ID NO: 23, and having equivalent or increased expression in a mammalian host cell relative to expression of nucleotides 97-753 of SEQ ID NO: 7 in the same host cell, wherein the polynucleotide does not have the nucleic acid sequence of nucleotides 97-753 of SEQ ID NO: 7.

Further aspects of the invention provide recombinant expression vectors, host cells, populations of cells, and pharmaceutical compositions relating to the synMMAB polynucleotides of the invention.

Still further aspects of the invention provide methods of treating a disease or condition mediated by MMAB enzyme.

Another aspect of the invention provides a method of detecting the presence of a synthetic MMAB polynucleotide (synMMAB) in a biological sample from a mammal, the method comprising: (a) obtaining at least one test sample comprising isolated nucleic acid from a biological sample from a mammal; (b) contacting any of the inventive synthetic polynucleotide described herein with the at least one test sample under conditions allowing for a complex to form between the synthetic polynucleotide and the isolated nucleic acid of the test sample; (c) detecting the complex; and (d) comparing a presence of the complex in the at least one test sample with an absence of complex from a negative sample that lacks the synthetic polynucleotide, wherein detection of the complex is indicative of the presence of the synthetic polynucleotide in the biological sample from the mammal.

### BRIEF DESCRIPTION OF THE SEVERAL VIEWS OF THE DRAWINGS

FIG. 1A is a map of the electrophoresis PCR products of MMAB T1-4, T1-5, and T1-8 that used PCR primers flanking the site for the guide RNA that was used to engineer mutations in the MMAB gene.

FIG. 1B is an alignment of the sequences of MMAB T1-NC, MMAB T1-4, MMAB T1-5, and MMAB T1-8. The gRNA sites are shaded in black and INDEL mutations are marked by arrows.

FIG. 1C is an image of a western blot showing the level of MMAB expression in HEK 293 MMAB KO T1-4 cells (lane 1), HEK 293 MMAB KO T1-8 cells (lane 2), HEK 293 MMAB T1-5 cells (lane 3), and parental control HEK 293 cells (lane 4).

FIGS. 2A-2E show an alignment of MMAB polynucleotides.

FIG. 2F is a phylogenetic tree of MMAB polynucleotides.

FIGS. 3A-3B are a map of the single-stranded AAV cassette (FIG. 3A) and the self-complementary AAV cassette (FIG. 3B).

FIGS. 4A-4C are images of western blots for expression of MMAB and bar graphs showing the results of western blot analysis in HEK 293 MMAB KO T1-8 cells transfected with no plasmid (T1-8-0), pAAV EF1L MMAB WT plasmid (T1-8-1), pAAV EF1L synMMAB1 (T1-8-2), pAAV EF1L synMMAB2 plasmid (T1-8-3), pscAAV EF1s MMAB WT BGH plasmid (T1-8-4), pscAAV EF1s MMAB WT RGB plasmid (T1-8-5), pscAAV EF1s synMMAB1 plasmid (T1-8-6), pscAAV EF1s synMAB2 plasmid (T1-8-7), pscAAV EF1s synMMAB3 plasmid (T1-8-8), pscAAV EF1s synMAB4 plasmid (T1-8-9), pscAAV EF1s synMMAB5 plasmid (T1-8-10), and pscAAV EF1s synMMAB6 plasmid (T1-8-11), compared to HEK293 MMAB WT cells when using 2.5 pg of plasmid (FIG. 4A), 1 μg of plasmid (FIG. 4B), and 0.25 pg of plasmid (FIG. 4C).

FIGS. 5A-5C are images of western blots for expression of MMAB and bar graphs showing the results of western blot analysis in HEK 293 MMAB KO T1-4 cells transfected with no plasmid (T1-4-0), pAAV EF1L MMAB WT plasmid (T1-4-1), pAAV EF1L synMAAB1 (T1-4-2), pAAV EF1L synMMAB2 plasmid (T1-4-3), pscAAV EF1s MMAB WT BGH plasmid (T1-4-4), pscAAV EF1s MMAB WT RGB plasmid (T1-4-5), pscAAV EF1s synMMAB1 plasmid (T1-4-6), pscAAV EF1s synMMAB2 plasmid (T1-4-7), pscAAV EF1s synMMAB3 plasmid (T1-4-8), pscAAV EF1s synMMAB4 plasmid (T1-4-9), pscAAV EF1s synMMAB5 plasmid (T1-4-10), and pscAAV EF1s synMMAB6 plasmid (T1-4-11), compared to HEK293 MMAB WT when using 2.5 pg of plasmid (FIG. 5A), 1 μg of plasmid (FIG. 5B), and 0.25 μg of plasmid (FIG. 5C).

FIGS. 6A-6B are an image of a western blot for expression of MMAB (FIG. 6A) and a bar graph showing the results of western blot analysis in HEK 293 MMAB KO T1-8 cells given no treatment, or infected with AAV8 EF1L synMMAB2(ss) (striped bars) at moiety of infection (moi) doses of 5E+4, 1E+5, 5E+5, and 1E+6 or AAV8 EF1s synMMAB5(sc) (solid bars) at moiety of infection (moi) doses of 2.5E+4, 5E+4, 2.5E+5, and 5E+6, compared to HEK293 MMAB WT cells (FIG. 6B).

FIGS. 7A-7B are an image of a western blot for expression of MMAB (FIG. 7A) and a bar graph showing the results of western blot analysis in HEK 293 MMAB KO T1-8 cells given no treatment, or infected with AAV9 EF1L synMMAB2(ss) (striped bars) at moiety of infection (moi) doses of 5E+4, 1E+5, 5E+5, and 1E+6 or AAV9 EF1s synMMAB5(sc) (solid bars) at moiety of infection (moi) doses of 2.5E+4, 5E+4, 2.5E+5, and 5E+6, compared to HEK293 MMAB WT cells (FIG. 7B).

FIGS. 8A-8B are an image of a western blot for expression of MMAB (FIG. 10A) and a bar graph showing the results of western blot analysis in HEK 293 MMAB KO T1-4 cells given no treatment, or infected with AAV8 EF1L synMMAB2(ss) (striped bars) at moiety of infection (moi) doses of 5E+4, 1E+5, 5E+5, and 1E+6 or AAV8 EF1s synMMAB5(sc) (solid bars) at moiety of infection (moi) doses of 2.5E+4, 5E+4, 2.5E+5, and 5E+6, compared to HEK293 MMAB WT cells (FIG. 8B).

FIGS. 9A-9B are an image of a western blot for expression of MMAB (FIG. 9A) and a bar graph showing the results of western blot analysis in HEK 293 MMAB KO T1-4 cells (striped bars) and HEK 293 MMAB KO T1-8 cells (solid bars) given no treatment, or infected with AAV9 EF1L synMMAB2(ss) at moiety of infection (moi) doses of 5E+4, 1E+5, 5E+5, and 1E+6, compared to HEK293 MA/AB WT cells (FIG. 9B).

FIG. 10 presents a line graph showing the probability of survival for WT mice, heterozygous Mmab^{WT/Δ3-7} mice, homozygous Mmab^{Δ3/Δ3-7} mice, and homozygous Mmab^{Δ3-7/Δ3-7} mice systemically administered scAAV9 EF1s synMMAB5 on DOL1 (Mt+scAAV9 EF1a is synMMAB5). FIG. 10 presents data collected from mice treated with approximately 5e10 GC/pup.

FIG. 11 is a bar graph showing circulating plasma methylmalonic acid (MMA) levels in micromoles (uM) in MMAB^{WT/WT};Tg^{ROSA26-MCK-Mmab} mice, MMAB^{WT/R180W};Tg^{ROSA26-MCK-Mmab} mice, MMAB^{R180W/R180W};Tg^{ROSA26-MCK-Mmab} mice, and MMAB^{R180W/R180W};Tg^{ROSA26-MCK-Mmab} mice treated with the ssAAV9 EF1L synA/MAB2 or scAAV9 EF1a is synMMAB5 vectors at a time point in young adulthood (circle) and at a time point two weeks later or two weeks after infection.

FIG. 12 is a line graph showing results of a 1-¹³C propionate oxidation in MMAB^{WT/WT};Tg^{ROSA26-MCK-Mmab} mice (circle), MMAB^{R180W/R180W};Tg^{ROSA26-MCK-Mmab} mice (square), and MMAB^{R180W/R180W};Tg^{ROSA26-MCK-Mmab} mice treated with the ssAAV9 EF1L synMMAB2 (upward triangle) or scAAV9 EF1s synMMAB5 (downward triangle) vectors two weeks after infection.

FIGS. 13A-13B is an image of a western blot (WB) for expression of MMAB and a bar graph showing the results of western blot analysis in the liver of three MMAB^{WT/WT};Tg^{ROSA26-MCK-Mmab} mice, three MMAB^{WT/R180W};Tg^{ROSA26-MCK-Mmab} mice, three MMAB^{R180W/R180W}:Tg^{ROSA26-MCK-Mmab} mice, three MMAB^{R180W/R180W}:Tg^{ROSA26-MCK-Mmab} mice treated with the ssAAV9 EF1L synMMAB2, and three MMAB^{R180W/R180W};Tg^{ROSA26-MCK-Mmab} mice treated with the scAAV9 EF1s synMMAB5 vectors (FIG. 13A), and a bar graph showing the same western blot analysis with the data averaged for three mice in each group (FIG. 13B).

FIG. 13C is a bar graph showing the results of RT-qPCR analysis of MMAB mRNA expression levels in the liver of MMAB^{WT/WT};Tg^{ROSA26-MCK-Mmab} mice(circle), MMAB^{WT/R180W};Tg^{ROSA26-MCK-Mmab} mice(circle), MMAB^{R180W/R180W};Tg^{ROSA26-MCK-Mmab} mice, and MMAB^{R180W/R180W};Tg^{ROSA26-MCK-Mmab} mice treated with the ssAAV9 EF1L synMMAB2 or scAAV9 EF1s synMMAB5 vectors (square) compared to endogenous levels of mouse Mmab mRNA (circles).

FIGS. 14A-14B is an image of a western blot for expression of MMAB and a bar graph showing the results of western blot analysis in the kidney of three MMAB^{WT/WT};Tg^{ROSA26-MCK-Mmab} mice, three MMAB^{WT/R180W};Tg^{ROSA26-MCK-Mmab} mice, three MMAB^{R180W/R180W};Tg^{ROSA26-MCK-Mmab} mice, three MMAB^{R180W/R180W};Tg^{ROSA26-MCK-Mmab} mice treated with the ssAAV9 EF1L synMMAB2, and three MMAB^{R180W/R180W};Tg^{ROSA26-MCK-Mmab} mice treated with the scAAV9 EF1s synMMAB5 vectors (FIG. 14A), and a bar graph showing the same western blot analysis with the data averaged for three mice in each group (FIG. 14B).

FIG. 14C is a bar graph showing the results of RT-qPCR analysis of MMAB mRNA expression levels in the kidney of MMAB^{WT/WT};Tg^{ROSA26-MCK-Mmab} mice(circle), MMAB^{WT/R180W};Tg^{ROSA26-MCK-Mmab} mice(circle), MMAB^{R180W/R180W};Tg^{ROSA26-MCK-Mmab} mice (circle), and MMAB^{R180W/R180W};Tg^{ROSA26-MCK-Mmab} mice treated with the ssAAV9 EF1L synMMAB2 or scAAV9 EF1s synMMAB5 vectors (square) compared to endogenous levels of mouse Mmab mRNA (circles).

FIG. 15 is an illustration of the process of ss MMAB AAV vector optimization for humans showing the changes in plasmid maps to develop the Final ss AAV cassette (pAAV EF1 MMAB Kan).

FIGS. 16A-16B are images of western blots bar graphs showing the results of western blot analysis for expression of MMAB in BEK 293 MMAB KO T1-4 cells (FIG. 16A) and BEK 293 MMAB KO T1-8 cells (FIG. 16B) for cells given no treatment, or transfected with pAAV EF1L synA MAB2, pAAV EF1L synMMAB2 KC0002, pAAV EF1L MMAB KAN, compared to BEK 293 MMAB WT cells.

FIG. 17 is an illustration of the process of sc MMAB AAV vector optimization for humans showing the changes in plasmid maps to develop the Final sc AAV cassette (pscAAV EF1a is MMAB Kan).

FIG. 18 is an image of a western blot showing the results of expression of MMAB in HEK 293 MMAB KO T1-4 cells given no treatment, or transfected with pAAV EF1L synMMAB2, pAAV EF1L MMAB Kan, pscAAV EF1s synMMAB5, pscAAV EF1s MMAB Kan, compared to HEK 293 MMAB WT cells.

FIG. 19 is a bar graph showing expression of MMAB in HEK 293 MMAB KO T1-4 cells given no treatment, or transfected with pAAV EF1L synMMAB2, pAAV EF1L MMAB Kan, pscAAV EF1s synMMAB5, pscAAV EF1s MMAB Kan, compared to HEK 293 MMAB WT cells.

FIG. 20A is a plot of data representing the survival of Mmab^{Δ3-7/Q228X} mice challenged with a diet consisting of a 10-fold increase in Isoleucine and Valine with ( [symbol] ) and without (•) treatment with the AAV9.EF1 LMMAB Kan vector. FIG. 20B is a plot of data representing the pMMA levels measured from Mmab^{Δ3-7/Q228X} mice, Mmab^{Q228X/Q228X} mice, and Mmab^{Δ3-7/Δ3-7};Tg^{MCK-Mmab} (C57BL/6) mice challenged with the high Isoleucine/Valine diet with and without treatment the AAV9.EF1 LMMAB Kan vector.

FIGS. 21A-21C present data concerning the phenotype of the Mmab^{Δ3-7/Δ3-7} (C57BL/6× FVB/N) mouse strain. FIG. 21A presents a survivability probability plot comparing Mmab^{Δ3-7/Δ3-7} (C57BL/6× FVB/N) homozygous mice with heterozygous mice and wild-type mice. FIG. 21B presents data concerning the average mass of Mmab^{Δ3-7/Δ3-7} (C57BL/6× FVB/N) homozygous mice, heterozygous mice, and wild-type mice measured between 22 and 26 days old; data for males are presented in the left column, those for females are presented in the right column. FIG. 21C presents data concerning the pMMA levels for Mmab^{Δ3-7/Δ3-7} (C57BL/6× FVB/N) homozygous mice, heterozygous mice, and wild-type mice.

FIG. 22 presents graphs presenting data concerning the metabolic response in Mmab^{Δ3-7/Δ3-7} mice as compared to wild-type mice and after scAAV9 treatment. Data concerning pMMA are presented in the left panel, while data 1-¹³C propionate oxidation are presented in the right panel. The vectors employed were those depicted in FIG. 3, which were delivered at approximately 5E+10 viral genomes (VG)/pup. Data were obtained six months for wild-type mice and post-neonatal treatment of Mmab^{Δ3-7/Δ3-7} mice with scAAV9, except those for untreated Mmab^{Δ3-7/Δ3-7} mice, which were collected at 25 days of age.

FIG. 23 presents images demonstrating the histopathology in Mmab^{Δ3-7/Δ3-7} mouse liver (middle panel), as compared to wild-type mice (left panel) and after neonatal scAAV9 MMAB gene therapy administered to Mmab^{Δ3-7/Δ3-7} mice (right panel). Data for the untreated Mmab^{Δ3-7/Δ3-7} mice and wild-type mice were collected at 45 days of age, while those for the treated Mmab^{Δ3-7/Δ3-7} mice were collected at 51 days of age.

FIG. 24 presents data concerning the levels of MMAB expression seen in the liver, heart, kidney, and muscle tissue from untreated Mmab^{Δ3-7/Δ3-7} mice, as compared to wild-type mice and after neonatal scAAV9 MMAB gene therapy administered to Mmab^{Δ3-7/Δ3-7} mice. Data were obtained six months for wild-type mice and post-neonatal treatment of Mmab^{Δ3-7/Δ3-7} mice with scAAV9, while those for untreated Mmab^{Δ3-7/Δ3-7} mice, which were collected at 25 days of age.

FIGS. 25A-25D present images of tissue obtained from Mmab^{Δ3-7/Δ3-7}(C57BL/6×FVB/N) mice following neonatal treatment with scAAV9 EF1s synMMAB5 (5E10 GC/pup) and probed with synMMAB5 RNA in an RNA in situ hybridization study (RNAscope). FIG. 25A depicts brain, liver, heart, kidney, muscle, spleen. FIG. 25B depicts liver tissue; FIG. 25C depicts heart tissue; and FIG. 25D depicts kidney tissue.

FIGS. 26A-26F present images of Western blots for expression of MMAB (FIGS. 26A, 26C, and 26E, top panel: AAV9 EF1L MMAB Kan (ss); bottom panel: AAV9 EF1s MMAB Kan (sc)) and bar graphs (FIGS. 26B, 26D, and 26F) showing the results of western blot analysis in HEK 293 MMAB KO T1-8 cells (FIGS. 26A-26D) or HEK 293 MMAB KO T1-4 cells (FIGS. 26E and 26F) given no treatment, or infected with AAV9 EF1L MMAB Kan (ss) or AAV9 EF1s MMAB Kan (sc) at moiety of infection (moi) doses of 5E+4, 1E+5, 5E+5, and 1E+6 (FIGS. 26A and 26B), or AAV9 EF1L MMAB Kan (ss) at moiety of infection (moi) doses of 5E+4, 1E+5, 5E+5 and 1E+6, and AAV9 EF1s MMAB Kan (sc) at moiety of infection (moi) doses of 1E+5, 2E+5, 1E+6, and 2E6 (FIGS. 26C, 26D, 26E, 26F), compared to HEK293 MMAB WT cells. For the Western blotting, total protein was loaded at 12.5 μg/lane (FIGS. 26A and 26B) or 25p g/lane (FIGS. 26C, 26D, 26E and 26F). The vectors employed in the experiments leading to FIGS. 26A and 26B were constructed independently of those employed for FIGS. 26C-26F.

FIGS. 27A and 27B are line graphs showing the probability of survival for WT mice (+/+), heterozygous Mmab^{WT/Δ3-7} ((C57BL/6× FVB/N)) mice (+/−), homozygous Mmab^{Δ3-7/Δ3-7} ((C57BL/6× FVB/N)) mice (−/−), and homozygous Mmab^{Δ3-7/Δ3-7} mice systemically administered AAV9.EF1L MMAB Kan (ss) or scAAV9.EF1s MMAB Kan (sc) on DOL1 or at wean. The vectors employed in the experiment leading to FIG. 27A were constructed independently of that employed for FIG. 27B.

FIGS. 28A and 28B present data concerning the metabolic response in Mmab^{Δ3-7/Δ3-7} (C57BL/6× FVB/N) mice as compared to heterozygotes and wild-type mice and to the homozygotes after treatment with AAV9.EF1L MMAB Kan (ssAAV9) or scAAV9.EF1s MMAB Kan (scAAV9). Data concerning pMMA are presented in FIG. 28A; data concerning FGF21 levels are presented in FIG. 28B.

FIGS. 29A and 29B present data concerning the expression of MMAB in the liver (FIG. 29A) and kidney (FIG. 29B) tissue of Mmab^{Δ3-7/Δ3-7} (C57BL/6× FVB/N) mice as compared to heterozygotes and wild-type mice and to the homozygotes after treatment with AAV9.EF1L MMAB Kan (ss) or scAAV9.EF1s MMAB Kan (sc).

FIG. 30 is a graph presenting the survival probability of mouse strains including the Mmab^{Δ3-7/Δ3-7};Tg^{MCK-Mmab} (C57BL/6) strain.

FIG. 31 presents data concerning the pMMA levels for mouse strains containing the Tg^{MCK-Mmab} allele, including mice also homozygous for the Mmab^{Δ3-7} trait, heterozygous Mmab^{WT/Δ3-7} mice, and wild-type with respect to the Mmab^{Δ3-7} trait.

FIG. 32 presents data concerning the levels of MMAB expression seen in the liver, heart, kidney, and muscle tissue from untreated Mmab^{Δ3-7/Δ3-7};Tg^{MCK-Mmab} (C57BL/6) mice and in Tg^{MCK-Mmab} mice, which were either wild-type or heterozygous with respect to Mmab.

FIGS. 33A and 33B present data concerning plasma MMAB levels measured in untreated (pretreated) neonatal Mmab^{Δ3-7/Δ3-7};Tg^{MCK-Mmab} (C57BL/6) mice and for those mice 14 days, 30 days, 60 days and 90 days (as indicated in each figure, respectively) post-treatment with either AAV9.EF1L MMAB Kan (ss) or scAAV9.EF1s MMAB Kan (sc). The vectors employed in the experiment leading to FIG. 33A were constructed independently of that employed for FIG. 33B.

### DETAILED DESCRIPTION OF THE INVENTION

Aspects of the invention provide synthetic MMAB polynucleotides (synMMAB polynucleotides) that may provide any one or more of a variety of advantages. For example, the inventive synMMAB polynucleotides may provide enhanced expression in mammalian cells. Compared to wild-type (WT) human polynucleotides encoding MMAB enzyme, the inventive synMMAB polynucleotides are codon-optimized to enhance expression upon administration. The inventive synMMAB polynucleotides may provide any one or more of improved survival, reduction in metabolites, increased metabolic capacity to C13 propionic acid oxidation, and robust MMAB expression in the liver and kidney, e.g., in a mouse model of model of cb1B MMA.

The inventive synMMAB polynucleotides may, advantageously, be useful as a therapeutic, via viral or non-viral mediated gene delivery, to restore MMAB enzyme function in MMA patients. The inventive synMMAB polynucleotides may reduce or prevent metabolic instability and may ameliorate disease progression. The inventive synMMAB polynucleotides may also be useful for the in vitro production of MMAB enzyme for use in enzyme replacement therapy for MMA. Because the MMAB enzyme may also mediate other disorders of branched chain amino acid oxidation, the inventive synMMAB polynucleotides may be useful for treating conditions other than cb1B MMA.

The inventive synMMAB polynucleotides may provide increased expression of the MMAB gene relative to naturally occurring human MMAB sequences. In aspects, the inventive synMMAB polynucleotides were designed to not alter the naturally occurring human MMAB enzyme amino acid sequence. They were also designed to have any one or more of increased transcriptional, translational, and protein refolding efficacy. This design was accomplished by, e.g., any one or more of the evaluation of human codon biases, evaluation of GC, CpG, and negative GpC content, evaluation of the interaction between the codon and anti-codon, reducing or eliminating cryptic splicing sites and RNA instability motifs, and compatibility with the vector backbones.

As used herein, “MMAB” refers to human Metabolism Of Cobalamin Associated B enzyme (also referred to as “MMAB enzyme,” “cob(I)alamin adenosyltransferase,” or “5′-deoxyadenosylco(I)balamin transferase”). This protein catalyzes the final step in the conversion of vitamin B(12) into 5′-adenosylcobalamin (AdoCbl), a vitamin B12-containing coenzyme for methylmalonyl-CoA mutase. The amino acid sequence of human WT MMAB enzyme is set forth in SEQ ID NO: 21. The gene encoding naturally occurring human MMAB enzyme is referred to as MMAB (also referred to as “cbB”). The WT human MMAB nucleic acid sequence is set forth in SEQ ID NO: 7. The polynucleotides encoding synthetic MMAB enzyme are referred to as synAM/AB. Naturally occurring human MMAB is referred to as MMAB, while synthetic MMAB is designated as synA/IAB, even though the two are identical at the amino acid level.

The MMAB enzyme is processed after transcription, translation, and translocation into the mitochondrial inner space. During this importation and maturation process, the mitochondrial transit peptide, which corresponds to amino acids 1-32 of SEQ ID NO: 21, are removed to produce the mature MMAB enzyme, which is comprised of residues 33-250. The amino acid sequence of the wild type MMAB mitochondrial transit peptide is MAVCGLGSRLGLGSRLGLRGCFGAARLLYPRF (SEQ ID NO: 22). The amino acid sequence of the mature MMAB enzyme is SEQ ID NO: 23.

An aspect of the invention provides a synthetic MMAB polynucleotide (synMMAB) selected from the group consisting of: (a) a polynucleotide comprising the nucleic acid sequence of any one of SEQ ID NOs: 1-6; (b) a polynucleotide having a nucleic acid sequence with at least about 80% identity to the nucleic acid sequence of any one of SEQ ID NOs: 1-6 and encoding a polypeptide having an amino acid sequence at least about 90% identical to SEQ ID NO: 21, and having equivalent or increased expression in a mammalian host cell relative to expression of SEQ ID NO: 7 in the same host cell, wherein the polynucleotide does not have the nucleic acid sequence of SEQ ID NO: 7; and (c) a polynucleotide having a nucleic acid sequence with at least about 80% identity to the nucleic acid sequence of nucleotides 97-753 of any one of SEQ ID NOs: 1-6 and encoding a polypeptide having an amino acid sequence at least about 90% identical to SEQ ID NO: 23, and having equivalent or increased expression in a mammalian host cell relative to expression of nucleotides 97-753 of SEQ ID NO: 7 in the same host cell, wherein the polynucleotide does not have the nucleic acid sequence of nucleotides 97-753 of SEQ ID NO: 7.

In one aspect of the invention, codon optimization was employed to create a highly active and synthetic MMAB polynucleotide. Accordingly, in an aspect, the synthetic polynucleotide is codon-optimized. “Codon optimization” refers to the process of altering a naturally occurring polynucleotide sequence to enhance expression in the target organism, e.g., humans. Codon optimization involves determining the relative frequency of a codon in the protein-encoding genes in the human genome. For example, isoleucine can be encoded by AUU, AUC, or AUA, but in the human genome, AUC (47%), AUU (36%), and AUA (17%) are variably used to encode isoleucine in proteins. Therefore, in the proper sequence context, AUA would be changed to AUC to allow this codon to be more efficiently translated in human cells.

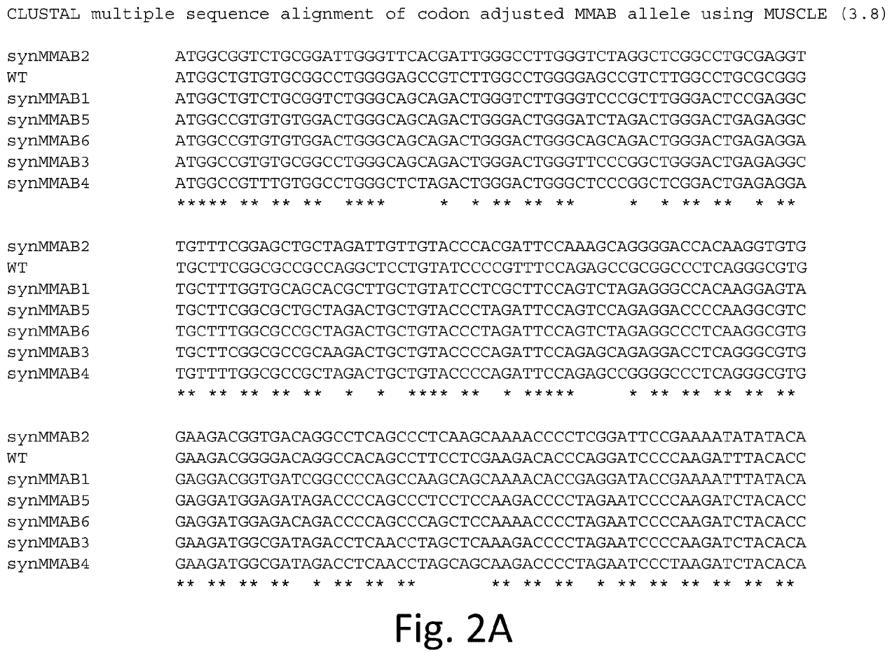

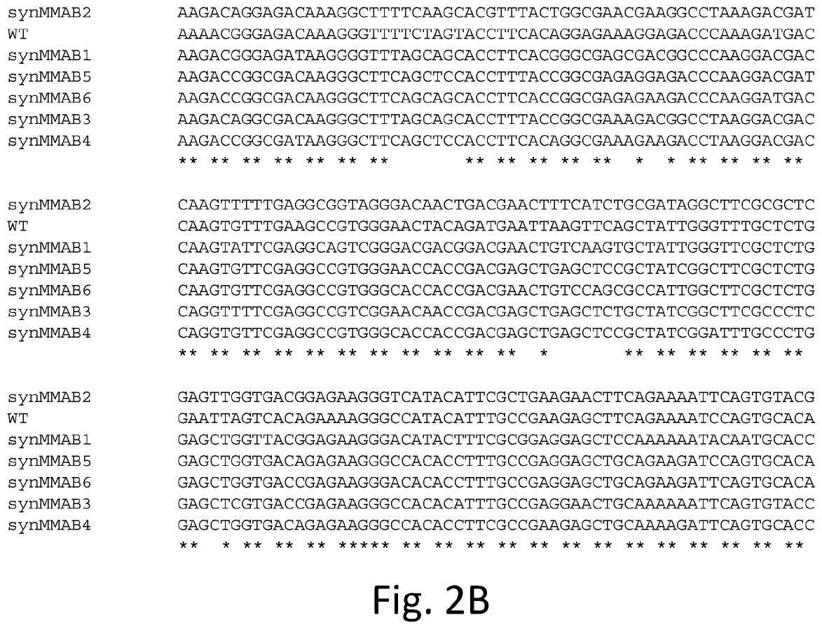

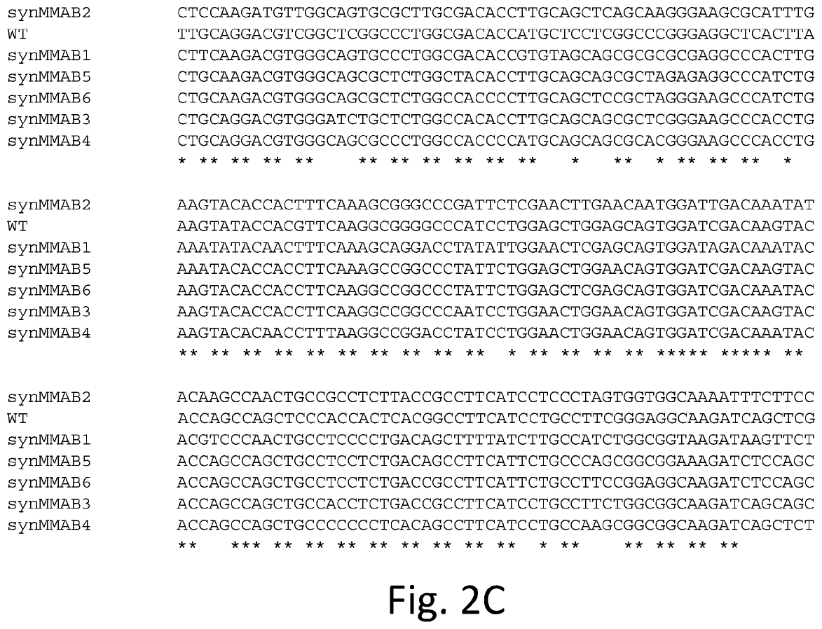

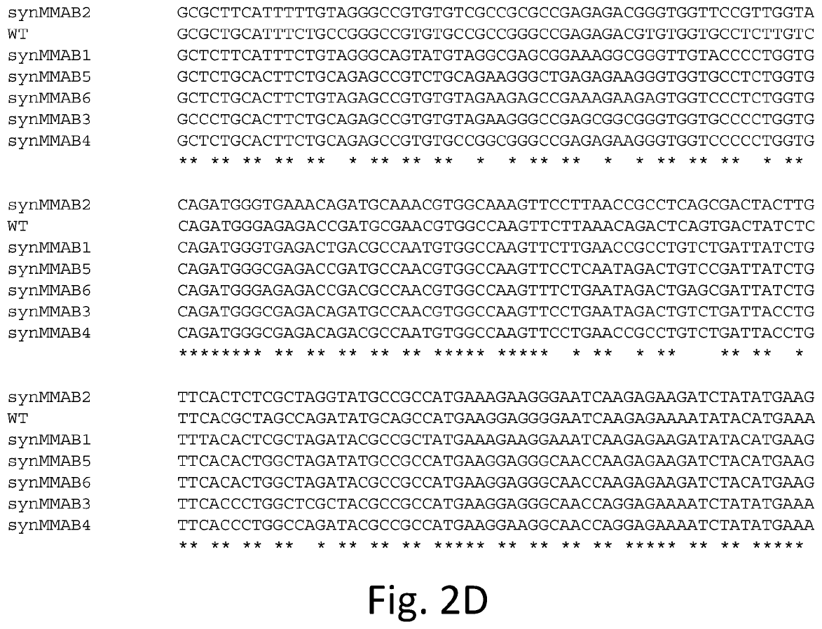

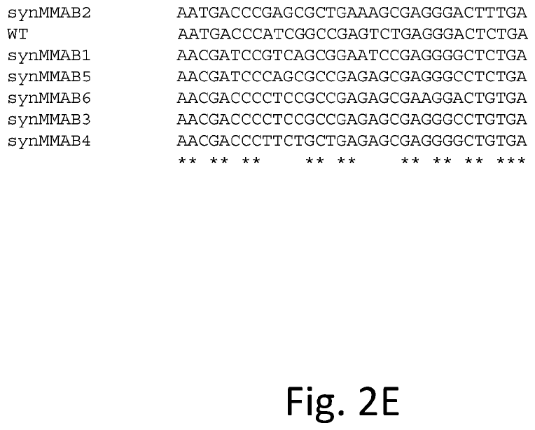

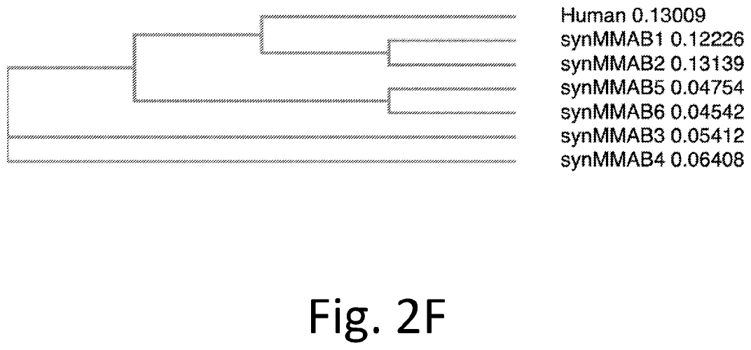

In the subject application, the human MMAB gene has been altered to replace codons that occur less frequently in human genes with those that occur more frequently and/or with codons that are frequently found in highly expressed human genes. A series of synthetic codon optimized and adjusted MMAB genes were developed (Table 2). An alignment (FIGS. 2A-2E) and phylogenetic analysis prepared with CLUSTAL W (FIG. 2F) reveals that the synMMAB polynucleotides are highly divergent at the nucleotide level while maintaining WT MMAB amino acid sequence identity.

Thus, as aspect of the invention comprises MMAB enzyme-encoding, synthetic polynucleotides comprising the nucleic acid sequence of any one of SEQ ID NOs: 1-6.

Another aspect of the invention provides a polynucleotide having a nucleic acid sequence with at least about 80% identity to the nucleic acid sequence of any one of SEQ ID NOs: 1-6 and encoding a polypeptide having an amino acid sequence at least about 90% identical to SEQ ID NO: 21 and having equivalent or increased expression in a mammalian host cell relative to expression of SEQ ID NO: 7 in the same host cell, wherein the polynucleotide does not have the nucleic acid sequence of SEQ ID NO: 7. Additional aspects of the invention provide a polynucleotide having a nucleic acid sequence with at least about 81%, at least about 82%, at least about 83%, at least about 84%, at least about 85%, at least about 86%, at least about 87%, at least about 88%, at least about 89%, 90%, at least about 91%, at least about 92%, at least about 93%, at least about 94%, at least about 95%, at least about 96%, at least about 97%, at least about 98%, or at least about 99% identity to the nucleic acid sequence of any one of SEQ ID NOs: 1-6 and encoding a polypeptide having an amino acid sequence at least about 90% identical to SEQ ID NO: 21, wherein the polynucleotide does not have the nucleic acid sequence of SEQ ID NO: 7.

Another aspect of the invention provides a polynucleotide having a nucleic acid sequence with at least about 80% identity to the nucleic acid sequence of nucleotides 97-753 of any one of SEQ ID NOs: 1-6 and encoding a polypeptide having an amino acid sequence at least about 90% identical to SEQ ID NO: 23, and having equivalent or increased expression in a mammalian host cell relative to expression of nucleotides 97-753 of SEQ ID NO: 7 in the same host cell, wherein the polynucleotide does not have the nucleic acid sequence of nucleotides 97-753 of SEQ ID NO: 7. Additional aspects of the invention provide a polynucleotide having a nucleic acid sequence with at least about 81%, at least about 82%, at least about 83%, at least about 84%, at least about 85%, at least about 86%, at least about 87%, at least about 88%, at least about 89%, 90%, at least about 91%, at least about 92%, at least about 93%, at least about 94%, at least about 95%, at least about 96%, at least about 97%, at least about 98%, or at least about 99% identity to the nucleic acid sequence of nucleotides 97-753 of any one of SEQ ID NOs: 1-6 and encoding a polypeptide having an amino acid sequence at least about 90% identical to SEQ ID NO: 23, wherein the polynucleotide does not have the nucleic acid sequence of nucleotides 97-753 of SEQ ID NO: 7.

In one aspect, the inventive synthetic polynucleotide encodes a polypeptide with 100% sequence identity to the naturally occurring, human MMAB enzyme (SEQ ID NO: 21) or the mature WT MMAB enzyme of SEQ ID NO: 23.

In another aspect, inventive synthetic polynucleotide encodes a polypeptide with at least about 90% identity to SEQ ID NO: 21 or the mature WT MMAB enzyme of SEQ ID NO: 23 and retaining the naturally occurring human MMAB protein function i.e., the capacity to catalyze the final step in the conversion of vitamin B(12) into adenosylcobalamin (AdoCbl). Additional aspects provide a synthetic polynucleotide that encodes a polypeptide with at least about 91%, at least about 92%, at least about 93%, at least about 94%, at least about 95%, at least about 96%, at least about 97%, at least about 98%, or at least about 99% identity to SEQ ID NO: 21 or the mature WT MMAB enzyme of SEQ ID NO: 23 and retaining the naturally occurring human MMAB protein function.

In one aspect, the MMAB enzyme encoded by the inventive polynucleotides retains at least about 90% of the naturally occurring human MMAB protein function, i.e., the capacity to catalyze the final step in the conversion of vitamin B(12) into adenosylcobalamin (AdoCbl). In another aspect, the encoded MMAB enzyme retains at least about 91%, at least about 92%, at least about 93%, at least about 94%, at least about 95%, at least about 96%, at least about 97%, at least about 98%, or at least about 99% of the naturally occurring human MMAB enzyme function. This enzyme function can be measured, for example, by measuring the ability to provide any one or more of improved survival, reduction in metabolites, and increased metabolic capacity to C13 propionic acid oxidation e.g., in a mouse model of model of cb1B MMA, for example, as described in the Examples below.

In some aspects, the synthetic polynucleotide exhibits improved expression in a mammalian host cell relative to the expression of naturally occurring human MMAB polynucleotide sequence. The improved expression is due to the polynucleotide comprising codons that have been optimized relative to the naturally occurring human MMAB polynucleotide sequence of SEQ ID NO: 7 (or nucleotides 97-753 of SEQ ID NO: 7). In one aspect, the synthetic polynucleotide has at least about 25%, at least about 30%, at least about 35%, at least about 40%, at least about 45%, at least about 50%, at least about 55%, at least about 60%, at least about 65%, at least about 70%, at least about 75%, or at least about 80% of less commonly used codons replaced with more commonly used codons. In additional aspects, the polynucleotide has at least about 85%, at least about 90%, or at least about 95% of less commonly used codons replaced with more commonly used codons, and demonstrate equivalent or enhanced expression of MMAB in a mammalian cell as compared to the expression of SEQ ID NO: 7 (or nucleotides 97-753 of SEQ ID NO: 7) in the same mammalian host cell.

In some aspects, the synthetic polynucleotide sequences of the invention provide equivalent or increased expression in a mammalian host cell relative to expression of SEQ ID NO: 7 (or nucleotides 97-753 of SEQ ID NO: 7) (as demonstrated by expression of the polynucleotide of SEQ ID NO: 7 (or nucleotides 97-753 of SEQ ID NO: 7) in the same mammalian host cell). In some aspects, the inventive polynucleotide exhibits an increase in expression in a mammalian host cell relative to the expression SEQ ID NO: 7 (or nucleotides 97-753 of SEQ ID NO: 7) in the same mammalian host cell. In some aspects, the inventive polynucleotide preferably encodes a polypeptide that retains at least about 80% of the enhanced MMAB expression (as demonstrated by expression of the polynucleotide of any one of SEQ ID NOs: 1-6 (or nucleotides 97-753 of any one of SEQ ID NOs: 1-6) in a mammalian host cell). In additional aspects, the polypeptide retains at least 85%, at least about 90%, at least about 95% or about 100% of the enhanced expression observed with the polynucleotide of any one of SEQ ID NOs: 1-6 (or nucleotides 97-753 of any one of SEQ ID NOs: 1-6) in a mammalian host cell. In aspects, the mammalian host cell is a human host cell or a mouse host cell. In some aspects, the mammalian host cell is a kidney cell or a liver cell from human or mouse.

In designing the synMMAB of the present invention, the following considerations were balanced. For example, the fewer changes that are made to the nucleotide sequence of SEQ ID NO: 7 (or nucleotides 97-753 of SEQ ID NO: 7), the less potential there is to alter the secondary structure of the sequence, which can have a significant impact on gene expression. The introduction of undesirable restriction sites was also reduced, facilitating the subcloning of synMMAB into the recombinant expression vector. However, a greater number of changes to the nucleotide sequence of SEQ ID NO: 7 (or nucleotides 97-753 of SEQ ID NO: 7) allows for more convenient identification of the translated and expressed message, e.g. mRNA, in vivo. Additionally, a greater number of changes to the nucleotide sequence of SEQ ID NO: 7 (or nucleotides 97-753 of SEQ ID NO: 7) provides for increased likelihood of greater expression. These considerations were balanced when arriving at the nucleic acid sequences of SEQ ID NO: 1-6 (or nucleotides 97-753 of any one of SEQ ID NOs: 1-6). The polynucleotide sequences encoding synMMAB allow for increased expression of the synMMAB polynucleotide relative to naturally occurring human MMAB polynucleotides. Because the sequences are novel, they may facilitate detection using nucleic acid-based assays.

MMAB has a total of 250 amino acids and synMMAB contains approximately 250 codons corresponding to said amino acids. In SEQ ID NOs: 1-6, codons were changed from that of the natural human MMAB. However, despite changes from SEQ ID NO: 7, SEQ ID NOs: 1-6 encode the amino acid sequence SEQ ID NO: 21 of WT, human MMAB. Codons for SEQ ID NOs: 1-6 were changed in accordance with the equivalent amino acid positions of SEQ ID NO: 21. In this aspect, the amino acid sequence for natural human MMAB has been retained.

Similarly, in nucleotides 97-753 of SEQ ID NOs: 1-6, codons were changed from that of the natural human AMAB. However, despite changes from nucleotides 97-753 of SEQ ID NO: 7, nucleotides 97-753 of SEQ ID NOs: 1-6 encode the amino acid sequence SEQ ID NO: 23 of WT, mature human MMAB. Codons for nucleotides 97-753 of SEQ ID NOs: 1-6 were changed in accordance with the equivalent amino acid positions of SEQ ID NO: 23. In this aspect, the amino acid sequence for natural human mature MMAB has been retained.

It can be appreciated that partial reversion of the designed synMMAB to codons that are found in MMAB can be expected to result in nucleic acid sequences that, when incorporated into appropriate vectors, can also exhibit the desirable properties of any one or more of SEQ ID NOs: 1-6 (or nucleotides 97-753 of any one of SEQ ID NOs: 1-6). For example, such partial reversion or hybrid variants can have MMAB expression from a vector inserted into an appropriate host cell that is equivalent to that of any one or more of SEQ ID NOs: 1-6 (or nucleotides 97-753 of any one of SEQ ID NOs: 1-6). For example, aspects of the invention include nucleic acids in which at least about 1 altered codon, at least about 2 altered codons, at least about 3, altered codons, at least about 4 altered codons, at least about 5 altered codons, at least about 6 altered codons, at least about 7 altered codons, at least about 8 altered codons, at least about 9 altered codons, at least about 10 altered codons, at least about 11 altered codons, at least about 12 altered codons, at least about 13 altered codons, at least about 14 altered codons, at least about 15 altered codons, at least about 16 altered codons, at least about 17 altered codons, at least about 18 altered codons, at least about 20 altered codons, at least about 25 altered codons, at least about 30 altered codons, at least about 35 altered codons, at least about 40 altered codons, at least about 50 altered codons, at least about 55 altered codons, at least about 60 altered codons, at least about 65 altered codons, at least about 70 altered codons, at least about 75 altered codons, at least about 80 altered codons, at least about 85 altered codons, at least about 90 altered codons, at least about 95 altered codons, at least about 100 altered codons, at least about 110 altered codons, at least about 120 altered codons, at least about 130 altered codons, at least about 130 altered codons, at least about 140 altered codons, at least about 150 altered codons, at least about 160 altered codons, at least about 170 altered codons, or at least about 180 altered codons, in any one of SEQ ID NOs: 1-6 (or nucleotides 97-753 of any one of SEQ ID NOs: 1-6) are reverted to native codons according to SEQ ID NO: 7 (or nucleotides 97-753 of SEQ ID NO: 7), and having equivalent or increased expression in a mammalian host cell as compared to expression of any one of SEQ ID NOs: 1-6 (or nucleotides 97-753 of any one of SEQ ID NOs: 1-6) in the same mammalian host cell. Alternately, at least about 1%, 2%, 3%, 4%, 5%, 10%, 15%, 20%, 25%, 30%, 35%, 40%, 45%, 50%, 55%, 60%, 65%, 70%, or 75% of the altered codon positions in any one of SEQ ID NOs: 1-6 (or nucleotides 97-753 of any one of SEQ ID NOs: 1-6) are reverted to the native sequence according to SEQ ID NO: 7 (or nucleotides 97-753 of SEQ ID NO: 7), and having equivalent or increased expression in a mammalian host cell as compared to expression of any one of SEQ ID NOs: 1-6 (or nucleotides 97-753 of any one of SEQ ID NOs: 1-6) in the same mammalian host cell.

The exact differences between SEQ ID NOs: 1-6 and SEQ ID NO: 7 demonstrate that the synMMAB polynucleotides are distinct from wild type human MMAB. Table 3 depicts the pairwise nucleotide identity between SEQ ID NOs: 1-6 and SEQ ID NO: 7. The values range between 73.1% and 77.8%. The exact positions of variation are depicted in a base-by-base comparison in FIGS. 2A-2E vs Table 3, and a phylogenetic analysis presented in FIG. 2F further demonstrates that SEQ ID NOs: 1-6 are distinct.

In some aspects, polynucleotides of the present invention do not share 100% identity with SEQ ID NO: 7 (or nucleotides 97-753 of SEQ ID NO: 7). In other words, in some aspects, polynucleotides having 100% identity with SEQ ID NO: 7 (or nucleotides 97-753 of SEQ ID NO: 7) are excluded from the aspects of the present invention.

In one aspect of a synthetic polynucleotide according to the invention, the nucleic acid sequence is a DNA sequence (e.g., a cDNA sequence). In another aspect, the nucleic acid sequence is a RNA sequence or peptide-modified nucleic acid sequence.

In another aspect, the invention is directed to a recombinant expression vector comprising any of the synMMAB polynucleotides described herein. In aspects of the invention, the recombinant expression vector may be single-stranded or self-complementary.

In an aspect of the invention, the recombinant expression vector may be a viral vector, such as a lentiviral vector, a retroviral vector, an alphaviral vector, a vaccinial viral vector, an adenoviral vector, a herpes viral vector, a fowl pox viral vector, or an adeno-associated viral (AAV) vector. While, in the Examples presented herein, studied vectors are AAV vectors derived from strains or serotypes 8 or 9 (AAV8 or AAV9 vectors), when the inventive vector is an AAV vector, such need not be limited to these exemplary strains or serotypes but can instead be from any suitable strain or serotype (e.g., AAV1, AAV2, AAV3, AAV4, AAV5, AAV6, AAV7, AAV8, AAV9, AAV10, AAV11, AAV12, AAV44.9, AAAV, BAAV, and the like). In other aspects of the invention, the recombinant expression vector may be a non-viral vector, such as plasmid DNA, liposome-DNA complex (lipoplexes), or polymer-DNA complex (polyplexes).

In certain aspects, the recombinant expression vector comprises an expression cassette. The expression cassette may, for example, include any one or more of a 5′ inverted terminal repeat (ITR), a promoter, one or more introns, a 5′ untranslated region (Kozak sequence), any of the inventive synMMAB polynucleotides described herein, an mRNA stability element, such as the WPRE (woodchuck post-transcriptional regulatory element) or HPRE (hepatitis B derived post-translational response element), a polyadenylation signal (e.g., bovine growth hormone polyadenylation signal (BGH A) or the rabbit beta-globin polyadenylation signal (rBGA)), CpG nucleotides, and a 3′ ITR.

In another aspect of the recombinant expression vector according to the invention, the synthetic polynucleotide is operably linked to an expression control sequence (e.g., a promoter). The promoter may vary with the type of viral recombinant expression vector used. For example, the promoter may be a viral promoter. Viral promoters include, for example, the ubiquitous cytomegalovirus immediate early (CMV-IE) promoter, the chicken beta-actin (CBA) promoter, the simian virus 40 (SV40) promoter, the Rous sarcoma virus long terminal repeat (RSV-LTR) promoter, the Moloney murine leukemia virus (MoMLV) LTR promoter, and other retroviral LTR promoters. In a preferred aspect, the promoter is the EF1S (elongation factor 1 promoter, short) promoter or the EF1L (elongation factor 1 promoter, long) promoter.

In one specific aspect, the inventive synMMAB polynucleotide could be placed under the transcriptional control of a ubiquitous or tissue-specific promoter. The use of a tissue-specific promoter can restrict unwanted MMAB expression, as well as facilitate persistent MMAB expression. The inventive synMMAB polynucleotide could then be delivered into the systemic circulation, portal vein, or directly injected into a tissue or organ, such as the liver or kidney. In an aspect of the invention, the recombinant expression vector is configured for expression of the synMMAB polynucleotide in one or both of hepatic tissue and renal tissue. In addition to the liver or kidney, the brain, pancreas, eye, heart, lungs, bone marrow, and muscle may constitute targets for therapy. Other tissues or organs may be additionally contemplated as targets for therapy.

Tissue-specific promoters include, without limitation, Apo A-I, ApoE, hAAT, transthyretin, liver-enriched activator, albumin, PEPCK, and RNAPr promoters (liver), PAI-1, ICAM-2 (endothelium), MCK, SMC α-actin, myosin heavy-chain, and myosin light-chain promoters (muscle), cytokeratin 18, CFTR (epithelium), GFAP, NSE, Synapsin I, Preproenkephalin, dβH, prolactin, and myelin basic protein promoters (neuronal), and ankyrin, α-spectrin, globin, HLA-DRα, CD4, glucose 6-phosphatase, and dectin-2 promoters (erythroid).

Regulatable promoters (for example, ligand-inducible or stimulus-inducible promoters) are also contemplated for expression constructs according to the invention. The inventive recombinant expression vectors can be designed for either transient expression, for stable expression, or for both. Also, the recombinant expression vectors can be made for constitutive expression or for inducible expression.

In yet another aspect, the synMMAB polynucleotide could be used in ex vivo applications via packaging into a recombinant expression vector to create an integrating recombinant expression vector that could be used to permanently correct any cell type from a patient with MMAB deficiency. The synMMAB-transduced and corrected cells could then be used as a cellular therapy. Examples might include CD34+ stem cells, primary hepatocytes, or fibroblasts derived from patients with MMAB deficiency. Fibroblasts could be reprogrammed to other cell types using iPS methods well known to practitioners of the art. In yet another aspect, the synMMAB polynucleotide could be recombined using genomic engineering and editing techniques that are well known to practitioners of the art, such as ZFNs, TALENS and Cas/CRISPR, into the AMAB locus, a genomic safe harbor site, such as AAVS1, or into another advantageous location, such as into rDNA, the albumin locus, SERPINA 5 or 7, PCSK9, GAPDH, or a suitable expressed pseudogene.

In an aspect of the invention, the recombinant expression vector comprises the nucleic acid sequence of any one of SEQ ID NOs: 9-10 and 13-20.

Another aspect of the invention further provides a host cell comprising any of the polynucleotides or any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Examples of host cells are known in the art and include, for instance, DH5a E. coli cells, Chinese hamster ovarian cells, monkey VERO cells, COS cells, HEK293 cells, and the like. For purposes of amplifying or replicating the recombinant expression vector, the host cell is preferably a prokaryotic cell, e.g., a DH5a cell. For purposes of producing an MMAB enzyme, the host cell is preferably a mammalian cell. Most preferably, the host cell is a human cell. The host cell may be, for example, a kidney cell or a liver cell from human or mouse.

Also provided by an aspect of the invention is a population of cells comprising at least one host cell described herein. The population of cells can be a heterogeneous population comprising the host cell comprising any of the recombinant expression vectors described herein, in addition to at least one other cell, e.g., a host cell (e.g., a kidney or liver cell), which does not comprise any of the recombinant expression vectors. Alternatively, the population of cells can be a substantially homogeneous population, in which the population comprises mainly of host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one aspect of the invention, the population of cells is a clonal population comprising host cells comprising a recombinant expression vector as described herein.

The inventive synthetic polynucleotides, recombinant expression vectors, host cells (including populations thereof), all of which are collectively referred to as “inventive synMMAB material(s)” hereinafter, can be formulated into a composition, such as a pharmaceutical composition. In this regard, an aspect of the invention provides a pharmaceutical composition comprising any of the inventive synthetic polynucleotides, recombinant expression vectors, or host cells (including populations thereof), described herein, and a pharmaceutically acceptable carrier.

A pharmaceutical composition for treating an mammal by gene therapy may comprise a therapeutically effective amount of a recombinant expression vector comprising the synMMAB polynucleotide or a viral particle produced by or obtained from same. The pharmaceutical composition may be for human or non-human mammal usage. Typically, a physician will determine the actual dosage which will be most suitable for an individual mammal, and it will vary with the age, weight, and response of the particular individual mammal.

The composition may, in specific aspects, comprise a pharmaceutically acceptable carrier, diluent, excipient, or adjuvant. Such materials should be non-toxic and should not interfere with the efficacy of the synMMAB polynucleotide. Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington: The Science and Practice of Pharmacy, 23^{rd} Ed., Academic Press (2020).

As used herein, a “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In certain aspects, it may be preferable to include isotonic agents, for example, sugars, polyalcohols such as mannitol, sorbitol, or sodium chloride in the composition.

The choice of pharmaceutical carrier, excipient, or diluent can be selected with regard to the intended route of administration and standard pharmaceutical practice. The pharmaceutical compositions may comprise as, or in addition to, the carrier, excipient, or diluent any suitable binder(s), lubricant(s), suspending agent(s), coating agent(s), solubilizing agent(s), and other carrier agents that may aid or increase the viral entry into the target site (such as for example a lipid delivery system). For oral administration, excipients such as starch or lactose may be used. For parenteral administration, a sterile aqueous solution may be used, optionally containing other substances, such as salts or monosaccharides to make the solution isotonic with blood.

A pharmaceutical composition according to the invention may be administered alone or in combination with at least one other agent, such as a stabilizing compound, which may be administered in any sterile, biocompatible pharmaceutical carrier, including, but not limited to, saline, buffered saline, dextrose, and water. The pharmaceutical compositions may be administered to a patient alone, or in combination with other agents, modulators, or drugs (e.g., antibiotics).

The pharmaceutical composition may be in a variety of forms. These include, for example, liquid, semi-solid and solid dosage forms, such as liquid solutions (e.g., injectable and infusible solutions), dispersions or suspensions, tablets, pills, powders, liposomes and suppositories. Additional dosage forms contemplated include: in the form of a suppository or pessary; in the form of a lotion, solution, cream, ointment or dusting powder; by use of a skin patch; in capsules or ovules; in the form of elixirs, solutions, or suspensions; in the form of tablets or lozenges.

Routes of delivery of a synMMAB polynucleotide according to the invention may include, without limitation, injection (systemic or at target site), for example, intradermal, subcutaneous, intravenous, intraperitoneal, intraocular, subretinal, renal artery, hepatic vein, intramuscular injection; physical, including ultrasound(-mediated transfection), electric field-induced molecular vibration, electroporation, transfection using laser irradiation, photochemical transfection, gene gun (particle bombardment); parenteral and oral (including inhalation aerosols and the like).

Vehicles for delivery of a synthetic MMAB polynucleotide (synMMAB) according to the invention may include, without limitation, viral vehicles (for example, AAV, adenovirus, baculovirus, retrovirus, lentivirus, foamy virus, herpes virus, Moloney murine leukemia virus, Vaccinia virus, and hepatitis virus) and non-viral vehicles (for example, naked DNA, mini-circles, CELID, doggy-bone DNA, liposomes, ligand-polylysine-DNA complexes, nanoparticles, cationic polymers, including polycationic polymers such as dendrimers, synthetic peptide complexes, artificial chromosomes, and polydispersed polymers). Thus, dosage forms contemplated include injectables, aerosolized particles, capsules, and other oral dosage forms.

In another aspect, the invention comprises a method of treating a disease or condition mediated by MMAB enzyme. The disease or condition can, in one aspect, be MMA or an MMAB deficiency. In an aspect of the invention, the disease or condition is cb1B type MMA. This method may comprise administering to a mammal in need thereof a therapeutic amount of any of the inventive synthetic polynucleotides, recombinant expression vectors, host cells, populations of cells, or pharmaceutical compositions described herein. The MMAB enzyme is processed after transcription, translation, and translocation into the mitochondrial inner space.

The MMAB enzyme contains a mitochondrial transit peptide, SEQ ID: 22, that is removed to produce the mature MMAB enzyme SEQ ID: 23 in the mitochondrial matrix. In another aspect of this invention, the respective nucleic acids within nucleotides 1-96 of SEQ IDs: 1-6 may be used to generate heterologous transit peptides. Nucleotides 97-753 of SEQ ID NOs: 1-6 may be used to generate the mature MMAB enzyme that could be used for enzyme replacement therapy or as a source of processed MMAB that might be suitable for use in enzymology, to discover vitamin B12 (adosylcobalamin) analogues and inhibitors, and as the active component of enzyme replacement therapy.

Enzyme replacement therapy involves the administration of the functional enzyme (MMAB) to a mammal in a manner so that the enzyme administered will catalyze the reactions in the body that the mammal's own defective or deleted enzyme cannot. In enzyme replacement therapy, the defective enzyme can be replaced in vivo or repaired in vitro using the synthetic polynucleotide according to the invention. The functional enzyme molecule can be isolated or produced in vitro, for example. Enzyme replacement therapy may be accomplished by administration of the synthetic MMAB protein orally, sub-cutaneously, intra-muscularly, intravenously, or by other therapeutic delivery routes.

Accordingly, an aspect of the invention provides a method of treating a disease or condition mediated by MMAB enzyme, comprising: producing the MMAB enzyme, with or without the transit peptide, by expressing any of the inventive synthetic polynucleotides or recombinant expression vectors described herein by a host cell, and purifying the enzyme from the host cell; and administering to a mammal in need thereof the purified MMAB enzyme. The host cells may be as described herein with respect to other aspects of the invention. Methods of purifying enzymes are known in the art.

In another aspect, the invention includes the mature MMAB enzyme, e.g., of SEQ ID NO: 23, attached to a carrier, synthetic or heterologous mitochondrial leader sequence, charged or lipophilic small molecule to direct toward the mitochondria; conjugated or covalently modified to a peptide that targets the mitochondrial matrix; or encapsulated to deliver the mature MMAB to a subcellular organelle, cell type or tissue.

Methods for producing recombinant enzymes in vitro are known in the art. In vitro enzyme expression systems include, without limitation, cell-based systems (bacterial (for example, Escherichia coli, Corynebacterium, Pseudomonasfluorescens), yeast (for example, Saccharomyces cerevisiae, Pichia Pastoris), insect cell (for example, Baculovirus-infected insect cells, non-lytic insect cell expression), and eukaryotic systems (for example, Leishmania)) and cell-free systems (using purified RNA polymerase, ribosomes, tRNA, ribonucleotides). Viral in vitro expression systems are likewise known in the art. The enzyme isolated or produced according to the above-iterated methods exhibits, in specific aspects, at least about 90%, at least about 91%, at least about 92%, at least about 93%, at least about 94%, at least about 95%, at least about 96%, at least about 97%, at least about 98%, or at least about 99%, or about 100% homology to the naturally occurring human MMAB enzyme (with or without the transit peptide).

Gene therapy can involve in vivo gene therapy (direct introduction of the genetic material into the cell or body) or ex vivo gene transfer, which usually involves genetically altering cells prior to administration of the genetically altered cells. In one aspect, genome editing, or genome editing with engineered nucleases (GEEN) may be performed with the synMMAB polynucleotides of the present invention allowing synMMAB DNA to be inserted, replaced, or removed from a genome using artificially engineered nucleases. Any known engineered nuclease may be used such as Zinc finger nucleases (ZFNs), Transcription Activator-Like Effector Nucleases (TALENs), the CRISPR/Cas system, and engineered meganuclease re-engineered homing endonucleases. Alternately, the synMMAB polynucleotides of the present invention, in combination with a CASP/CRISPR, ZFN, or TALEN, can be used to engineer correction at the locus in a mammal's cell either in vivo or ex vivo, then, in one aspect, use that corrected cell, such as a fibroblast or lymphoblast, to create an iPS or other stem cell for use in cellular therapy.

Accordingly, an aspect of the invention provides a method of treating a disease or condition mediated by MMAB enzyme, comprising administering to a cell of a mammal in need thereof any of the inventive polynucleotides described herein, wherein the polynucleotide is inserted into the cell of the mammal via genome editing on the cell of the mammal using a nuclease selected from the group of ZFNs, TALENs, the clustered regularly interspaced short palindromic repeats (CRISPR/cas system) and meganuclease re-engineered homing endonucleases on a cell from the mammal. In an aspect of the invention, the method comprises administering the polynucleotide to an isolated cell of the mammal, and the method further comprises administering the cell to the mammal. In another aspect of the invention, the method comprises administering the polynucleotide to the cell of the mammal in vivo.

The inventive methods of treating a disease or condition mediated by MMAB enzyme may comprise administering a therapeutically effective amount of the synADAB material (or the genetically altered cell) to the mammal. A “therapeutically effective amount” refers to an amount effective, at dosages and for periods of time necessary, to achieve the desired therapeutic result. A therapeutically effective amount of the synMMAB material may vary according to factors such as the disease state, age, sex, and weight of the mammal, and the ability of the synMMAB material to elicit a desired response in the mammal. A therapeutically effective amount is also one in which any toxic or detrimental effects of the synMMAB material are outweighed by the therapeutically beneficial effects.

Dosage regimens may be adjusted to provide the optimum desired response (e.g., a therapeutic response). For example, a single bolus may be administered, several divided doses may be administered over time, or the dose may be proportionally reduced or increased as indicated by the exigencies of the therapeutic situation. It may be especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of the synMMAB material calculated to produce the desired therapeutic effect in association with a pharmaceutical carrier.

The term “treat,” as well as words stemming therefrom, as used herein, do not necessarily imply 100% or complete treatment. Rather, there are varying degrees of treatment of which one of ordinary skill in the art recognizes as having a potential benefit or therapeutic effect. In this respect, the inventive methods can provide any amount of any level of treatment of the disease or condition in a mammal. Furthermore, the treatment provided by the inventive method can include treatment of one or more conditions or symptoms of the disease or condition being treated. In another aspect, the invention is directed to the preclinical amelioration or rescue from the condition or disease state, for example, MMA, that the afflicted mammal exhibits. This may include symptoms, such as lethargy, lethality, metabolic acidosis, and biochemical perturbations, such as increased levels of methylmalonic acid in blood, urine, and body fluids.

The mammal referred to in the inventive methods can be any mammal. As used herein, the term “mammal” refers to any mammal, including, but not limited to, mammals of the order Rodentia, such as mice and hamsters, and mammals of the order Lagomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

Another aspect of the invention provides a method of detecting the presence of a synthetic MMAB polynucleotide (synMMAB) in a biological sample from a mammal. Such methods may be useful for any of a variety of applications, for example, detecting the presence of the synMAB polynucleotide in a biological sample from a mammal that has been administered any of the synMMAB materials as described herein.

The method may comprise obtaining at least one test sample comprising nucleic acid from a biological sample from a mammal. The biological sample may be a sample of any tissue from the mammal, for example, blood, kidney, liver, or any of the other tissues and organs described herein with respect to other aspects of the invention. In an aspect, the nucleic acid is isolated or purified from the biological sample to form the test sample. In another aspect, the test sample comprises the biological sample, and the nucleic acid in the biological sample is tested in situ.

The method may further comprise contacting any of the inventive synMMAB polynucleotides described herein with the at least one test sample under conditions allowing for a complex to form between the synMMAB polynucleotide and the nucleic acid of the test sample. In this regard, the method comprises contacting the nucleic acid of the test sample with the inventive synMMAB polynucleotide under conditions which allow the inventive synMMAB polynucleotide to specifically hybridize with the nucleic acid of the test sample as is known in the art. The method may comprise amplifying the inventive synMMAB polynucleotide and the nucleic acid of the test sample nucleic acid using any suitable type of polymerase chain reaction (PCR) as is known in the art.

In an aspect, the inventive synMAB polynucleotide further comprises a detectable label. The label may be any label suitable for detecting hybridization, e.g., a complex, of the inventive synMMAB polynucleotide with the nucleic acid of the test sample. Exemplary detectable labels may include any one or more of radioactive labels, non-radioactive labels, fluorescent labels, and chemiluminescent labels.

The method may further comprise detecting the complex. The complex may be detected using, for example, a radioactive label or a dye as is known in the art. In a preferred embodiment, the method comprises measuring light emitted from a fluorescent dye using, e.g., a laser. Detecting the complex may, optionally, further comprise measuring the amount of complex formed.

The method may further comprise comparing a presence of the complex in the at least one test sample with an absence of complex from a negative sample that lacks the inventive synMMAB polynucleotide. The presence of complex from the at least one test sample is indicative of the presence of the inventive synMMAB polynucleotide in the test sample and the absence of complex from the at least one test sample is indicative of the absence of the inventive synMMAB polynucleotide in the test sample. In an aspect of the invention, the method comprises determining a background level of signal generated by the label in the negative sample that lacks the inventive synMMAB polynucleotide and comparing the background level of signal with the level of signal detected in the test sample. A level of signal that is higher or lower in the test sample as compared to that measured in the negative sample may be indicative of the presence of the inventive synMMAB polynucleotide in the test sample.

The following examples further illustrate the invention but, of course, should not be construed as in any way limiting its scope.

### Example 1

This example demonstrates the development of HEK293 mutant MMAB knockout cell lines using CRISPR CAS9.

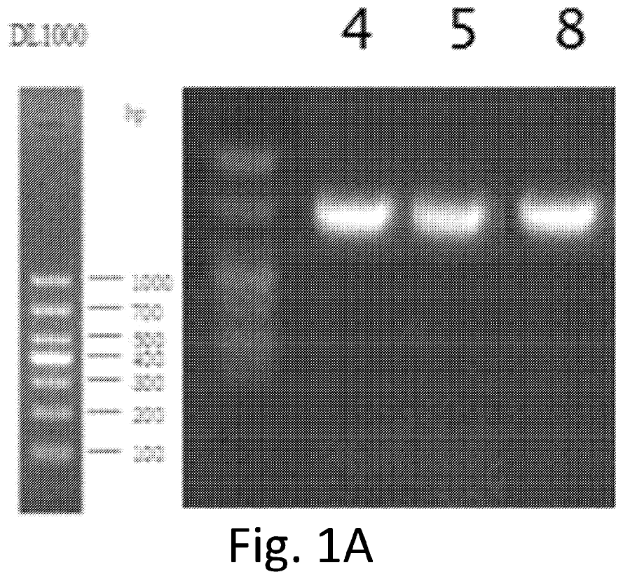

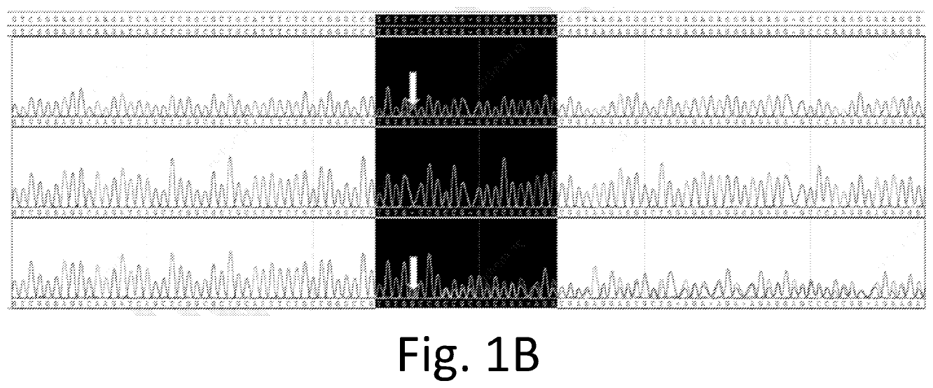

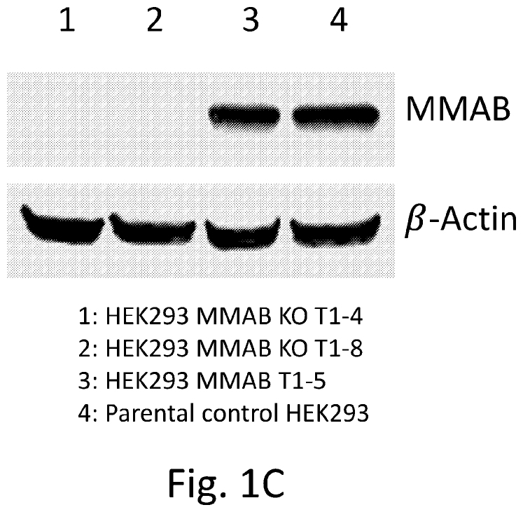

Using CRISPR gene editing, HEK293 cells were transfected with Cas9 and CRISPR guide RNAs obtained from GenScript USA Inc. and subjected to single cell cloning. After single cell cloning was completed, a wild type clone, T1-5, and two knockout clones T1-4 and T1-8 were further pursued and analyzed (Table 1). PCR was performed on T1-4, T1-5, and T1-8 followed by gel electrophoresis (FIG. 1A). Sequencing was also performed on T1-4, T1-5, and T1-8 and analyzed using CRISPR analysis tool (CAT) (FIG. 1). Clone T1-5 had no mutations, clone T1-4 possessed a homozygous mutation MMAB c.566dupG p.(Cys189TrpfsTer30), and Clone T1-8 possessed the compound genotype mutationMMAB c.566dupG p.(Cys189TrpfsTer30) and c.567_569del p.(Arg191del). Western blotting was performed for MMAB levels in HEK293T cells and HEK293 MA/AB clone T1-4 cells, HEK293 MMAB clone T1-5 cells, and HEK293 MA/AB clone T1-8 cells using 50 micrograms of cell lysate and Proteintech Ab (#11137-1-AP) at a dilution of 1:1000 (FIG. 1C). The HEK293 MA/AB clone T1-4 cells and HEK293 MMAB clone T1-8 cells both showed a lack of immunoreactive MMAB. These results suggest that the HEK293 A/JAB clone T1-4 and HEK293 MA/AB clone T1-8 cell lines harbor pathogenic mutations, with no immunoreactive enzyme MMAB.

TABLE 1

| Clone ID | INDELS | Genotype | Host Cell Line |
|---|---|---|---|
| Clone T1-4 | +1/+1 | Full allelic knockout | HEK293 |
| Clone T1-5 | 0/0 | Wildtype | HEK293 |
| Clone T1-8 | −2/+1 | Full allelic knockout | HEK293 |

In summary, these engineered cell lines lack MMAB expression and can be used to assess plasmid and AAV vectors designed to express MMAB. These knockout (KO) cell lines were employed in the experiments described below. A potency assay is presented for AAV8 and AAV9 vectors that follows the general approach: in a 6 well plate, variable doses of AAV8 or AAV9 are used to infect the MMAB T1-4 and/or T1-8 cells; the cells are incubated for 48 hours and then harvested; and the lysate is subjected to western analysis to detect MMAB.

### Example 2

This example demonstrates the development of synthetic codon optimized and adjusted MMAB genes.

A series of synthetic codon optimized and adjusted MMAB genes were developed (Table 2). A pairwise comparison of wild type MMAB with synMMAB polynucleotides was carried out. The percentage of nucleotide identity is listed in Table 3. A sequence alignment of wild type MMAB with synMMAB polynucleotides is shown in FIGS. 2A-2E. Analyses of wild type MMAB and synthetic MMAB polynucleotides (synMMAB) were carried out with CLUSTAL W for phylogenetic analysis (FIG. 2F). These analyses revealed that the synMMAB polynucleotides are highly divergent at the nucleotide level while maintaining wild-type MMAB amino acid sequence identity.

TABLE 2

| MMAB | |
|---|---|
| Gene | Sequence |
| synMMAB1-SEQ | ATGGCTGTCTGCGGTCTGGGCAGCAGACTGGGTCTTGGGTCCCGCTTG |
| ID NO: 1 | GGACTCCGAGGCTGCTTTGGTGCAGCACGCTTGCTGTATCCTCGCTTC |
| | CAGTCTAGAGGGCCACAAGGAGTAGAGGACGGTGATCGGCCCCAGCC |
| | AAGCAGCAAAACACCGAGGATACCGAAAATTTATACAAAGACGGGA |
| | GATAAGGGGTTTAGCAGCACCTTCACGGGCGAGCGACGGCCCAAGGA |
| | CGACCAAGTATTCGAGGCAGTCGGGACGACGGACGAACTGTCAAGTG |
| | CTATTGGGTTCGCTCTGGAGCTGGTTACGGAGAAGGGACATACTTTCG |
| | CGGAGGAGCTCCAAAAAATACAATGCACCCTTCAAGACGTGGGCAGT |
| | GCCCTGGCGACACCGTGTAGCAGCGCGCGCGAGGCCCACTTGAAATA |
| | TACAACTTTCAAAGCAGGACCTATATTGGAACTCGAGCAGTGGATAG |
| | ACAAATACACGTCCCAACTGCCTCCCCTGACAGCTTTTATCTTGCCAT |
| | CTGGCGGTAAGATAAGTTCTGCTCTTCATTTCTGTAGGGCAGTATGTA |
| | GGCGAGCGGAAAGGCGGGTTGTACCCCTGGTGCAGATGGGTGAGACT |
| | GACGCCAATGTGGCCAAGTTCTTGAACCGCCTGTCTGATTATCTGTTT |
| | ACACTCGCTAGATACGCCGCTATGAAAGAAGGAAATCAAGAGAAGAT |
| | ATACATGAAGAACGATCCGTCAGCGGAATCCGAGGGGCTCTGA |
| synMMAB2-SEQ | ATGGCGGTCTGCGGATTGGGTTCACGATTGGGCCTTGGGTCTAGGCTC |
| ID NO: 2 | GGCCTGCGAGGTTGTTTCGGAGCTGCTAGATTGTTGTACCCACGATTC |
| | CAAAGCAGGGGACCACAAGGTGTGGAAGACGGTGACAGGCCTCAGCC |
| | CTCAAGCAAAACCCCTCGGATTCCGAAAATATATACAAAGACAGGAG |
| | ACAAAGGCTTTTCAAGCACGTTTACTGGCGAACGAAGGCCTAAAGAC |
| | GATCAAGTTTTTGAGGCGGTAGGGACAACTGACGAACTTTCATCTGCG |
| | ATAGGCTTCGCGCTCGAGTTGGTGACGGAGAAGGGTCATACATTCGCT |
| | GAAGAACTTCAGAAAATTCAGTGTACGCTCCAAGATGTTGGCAGTGC |
| | GCTTGCGACACCTTGCAGCTCAGCAAGGGAAGCGCATTTGAAGTACA |
| | CCACTTTCAAAGCGGGCCCGATTCTCGAACTTGAACAATGGATTGACA |
| | AATATACAAGCCAACTGCCGCCTCTTACCGCCTTCATCCTCCCTAGTG |
| | GTGGCAAAATTTCTTCCGCGCTTCATTTTTGTAGGGCCGTGTGTCGCCG |
| | CGCCGAGAGACGGGTGGTTCCGTTGGTACAGATGGGTGAAACAGATG |
| | CAAACGTGGCAAAGTTCCTTAACCGCCTCAGCGACTACTTGTTCACTC |
| | TCGCTAGGTATGCCGCCATGAAAGAAGGGAATCAAGAGAAGATCTAT |
| | ATGAAGAATGACCCGAGCGCTGAAAGCGAGGGACTTTGA |
| synMMAB3-SEQ | ATGGCCGTGTGCGGCCTGGGCAGCAGACTGGGACTGGGTTCCCGGCT |
| ID NO: 3 | GGGACTGAGAGGCTGCTTCGGCGCCGCAAGACTGCTGTACCCCAGAT |
| | TCCAGAGCAGAGGACCTCAGGGCGTGGAAGATGGCGATAGACCTCAA |
| | CCTAGCTCAAAGACCCCTAGAATCCCCAAGATCTACACAAAGACAGG |
| | CGACAAGGGCTTTAGCAGCACCTTTACCGGCGAAAGACGGCCTAAGG |
| | ACGACCAGGTTTTCGAGGCCGTCGGAACAACCGACGAGCTGAGCTCT |
| | GCTATCGGCTTCGCCCTCGAGCTCGTGACCGAGAAGGGCCACACATTT |
| | GCCGAGGAACTGCAAAAAATTCAGTGTACCCTGCAGGACGTGGGATC |
| | TGCTCTGGCCACACCTTGCAGCAGCGCTCGGGAAGCCCACCTGAAGTA |
| | CACCACCTTCAAGGCCGGCCCAATCCTGGAACTGGAACAGTGGATCG |
| | ACAAGTACACCAGCCAGCTGCCACCTCTGACCGCCTTCATCCTGCCTT |
| | CTGGCGGCAAGATCAGCAGCGCCCTGCACTTCTGCAGAGCCGTGTGTA |
| | GAAGGGCCGAGCGGCGGGTGGTGCCCCTGGTGCAGATGGGCGAGACA |
| | GATGCCAACGTGGCCAAGTTCCTGAATAGACTGTCTGATTACCTGTTC |
| | ACCCTGGCTCGCTACGCCGCCATGAAGGAGGGCAACCAGGAGAAAAT |
| | CTATATGAAAAACGACCCCTCCGCCGAGAGCGAGGGCCTGTGA |
| synMMA4-SEQ | ATGGCCGTTTGTGGCCTGGGCTCTAGACTGGGACTGGGCTCCCGGCTC |
| ID NO: 4 | GGACTGAGAGGATGTTTTGGCGCCGCTAGACTGCTGTACCCCAGATTC |
| | CAGAGCCGGGGCCCTCAGGGCGTGGAAGATGGCGATAGACCTCAACC |
| | TAGCAGCAAGACCCCTAGAATCCCTAAGATCTACACAAAGACCGGCG |
| | ATAAGGGCTTCAGCTCCACCTTCACAGGCGAAAGAAGACCTAAGGAC |
| | GACCAGGTGTTCGAGGCCGTGGGCACCACCGACGAGCTGAGCTCCGC |
| | TATCGGATTTGCCCTGGAGCTGGTGACAGAGAAGGGCCACACCTTCGC |
| | CGAAGAGCTGCAAAAGATTCAGTGCACCCTGCAGGACGTGGGCAGCG |
| | CCCTGGCCACCCCATGCAGCAGCGCACGGGAAGCCCACCTGAAGTAC |
| | ACAACCTTTAAGGCCGGACCTATCCTGGAACTGGAACAGTGGATCGA |
| | CAAATACACCAGCCAGCTGCCCCCCCTCACAGCCTTCATCCTGCCAAG |
| | CGGCGGCAAGATCAGCTCTGCTCTGCACTTCTGCAGAGCCGTGTGCCG |
| | GCGGGCCGAGAGAAGGGTGGTCCCCCTGGTGCAGATGGGCGAGACAG |
| | ACGCCAATGTGGCCAAGTTCCTGAACCGCCTGTCTGATTACCTGTTCA |
| | CCCTGGCCAGATACGCCGCCATGAAGGAAGGCAACCAGGAGAAAATC |
| | TATATGAAAAACGACCCTTCTGCTGAGAGCGAGGGGCTGTGA |
| synMMAB5-SEQ | ATGGCCGTGTGTGGACTGGGCAGCAGACTGGGACTGGGATCTAGACT |
| ID NO: 5 | GGGACTGAGAGGCTGCTTCGGCGCTGCTAGACTGCTGTACCCTAGATT |
| | CCAGTCCAGAGGACCCCAAGGCGTCGAGGATGGAGATAGACCCCAGC |
| | CCTCCTCCAAGACCCCTAGAATCCCCAAGATCTACACCAAGACCGGCG |
| | ACAAGGGCTTCAGCTCCACCTTTACCGGCGAGAGGAGACCCAAGGAC |
| | GATCAAGTGTTCGAGGCCGTGGGAACCACCGACGAGCTGAGCTCCGC |
| | TATCGGCTTCGCTCTGGAGCTGGTGACAGAGAAGGGCCACACCTTTGC |
| | CGAGGAGCTGCAGAAGATCCAGTGCACACTGCAAGACGTGGGCAGCG |
| | CTCTGGCTACACCTTGCAGCAGCGCTAGAGAGGCCCATCTGAAATACA |
| | CCACCTTCAAAGCCGGCCCTATTCTGGAGCTGGAACAGTGGATCGACA |
| | AGTACACCAGCCAGCTGCCTCCTCTGACAGCCTTCATTCTGCCCAGCG |
| | GCGGAAAGATCTCCAGCGCTCTGCACTTCTGCAGAGCCGTCTGCAGAA |
| | GGGCTGAGAGAAGGGTGGTGCCTCTGGTGCAGATGGGCGAGACCGAT |
| | GCCAACGTGGCCAAGTTCCTCAATAGACTGTCCGATTATCTGTTCACA |
| | CTGGCTAGATATGCCGCCATGAAGGAGGGCAACCAAGAGAAGATCTA |
| | CATGAAGAACGATCCCAGCGCCGAGAGCGAGGGCCTCTGA |
| synMMAB6-SEQ | ATGGCCGTGTGTGGACTGGGCAGCAGACTGGGACTGGGCAGCAGACT |
| ID NO: 6 | GGGACTGAGAGGATGCTTTGGCGCCGCTAGACTGCTGTACCCTAGATT |
| | CCAGTCTAGAGGCCCTCAAGGCGTGGAGGATGGAGACAGACCCCAGC |
| | CCAGCTCCAAAACCCCTAGAATCCCCAAGATCTACACCAAGACCGGC |
| | GACAAGGGCTTCAGCAGCACCTTCACCGGCGAGAGAAGACCCAAGGA |
| | TGACCAAGTGTTCGAGGCCGTGGGCACCACCGACGAACTGTCCAGCG |
| | CCATTGGCTTCGCTCTGGAGCTGGTGACCGAGAAGGGACACACCTTTG |
| | CCGAGGAGCTGCAGAAGATTCAGTGCACACTGCAAGACGTGGGCAGC |
| | GCTCTGGCCACCCCTTGCAGCTCCGCTAGGGAAGCCCATCTGAAGTAC |
| | ACCACCTTCAAGGCCGGCCCTATTCTGGAGCTCGAGCAGTGGATCGAC |
| | AAATACACCAGCCAGCTGCCTCCTCTGACCGCCTTCATTCTGCCTTCC |
| | GGAGGCAAGATCTCCAGCGCTCTGCACTTCTGTAGAGCCGTGTGTAGA |
| | AGAGCCGAAAGAAGAGTGGTCCCTCTGGTGCAGATGGGAGAGACCGA |
| | CGCCAACGTGGCCAAGTTTCTGAATAGACTGAGCGATTATCTGTTCAC |
| | ACTGGCTAGATACGCCGCCATGAAGGAGGGCAACCAAGAGAAGATCT |
| | ACATGAAGAACGACCCCTCCGCCGAGAGCGAAGGACTGTGA |
| Wild | ATGGCTGTGTGCGGCCTGGGGAGCCGTCTTGGCCTGGGGAGCCGTCTT |
| type | GGCCTGCGCGGGTGCTTCGGCGCCGCCAGGCTCCTGTATCCCCGTTTC |
| Human | CAGAGCCGCGGCCCTCAGGGCGTGGAAGACGGGGACAGGCCACAGCC |
| MMAB | TTCCTCGAAGACACCCAGGATCCCCAAGATTTACACCAAAACGGGAG |
| cDNA-SEQ | ACAAAGGGTTTTCTAGTACCTTCACAGGAGAAAGGAGACCCAAAGAT |
| ID NO: 7 | GACCAAGTGTTTGAAGCCGTGGGAACTACAGATGAATTAAGTTCAGC |
| | TATTGGGTTTGCTCTGGAATTAGTCACAGAAAAGGGCCATACATTTGC |
| | CGAAGAGCTTCAGAAAATCCAGTGCACATTGCAGGACGTCGGCTCGG |
| | CCCTGGCGACACCATGCTCCTCGGCCCGGGAGGCTCACTTAAAGTATA |
| | CCACGTTCAAGGCGGGGCCCATCCTGGAGCTGGAGCAGTGGATCGAC |
| | AAGTACACCAGCCAGCTCCCACCACTCACGGCCTTCATCCTGCCTTCG |
| | GGAGGCAAGATCAGCTCGGCGCTGCATTTCTGCCGGGCCGTGTGCCGC |
| | CGGGCCGAGAGACGTGTGGTGCCTCTTGTCCAGATGGGAGAGACCGA |
| | TGCGAACGTGGCCAAGTTCTTAAACAGACTCAGTGACTATCTCTTCAC |
| | GCTAGCCAGATATGCAGCCATGAAGGAGGGGAATCAAGAGAAAATAT |
| | ACATGAAAAATGACCCATCGGCCGAGTCTGAGGGACTCTGA |

TABLE 3

| Polynucleotide 1 | Polynucleotide 2 | Percent nucleotide identity |
|---|---|---|
| MMAB | synMMAB1 | 73.1 |
| MMAB | synMMAB2 | 73.6 |
| MMAB | synMMAB3 | 77.8 |
| MMAB | synMMAB4 | 76.5 |
| MMAB | synMMAB5 | 76.9 |
| MMAB | synMMAB6 | 77.3 |

### Example 3

This example demonstrates the development of AAV 8 and AAV 9 vectors to express the synthetic MA/AB polynucleotides.

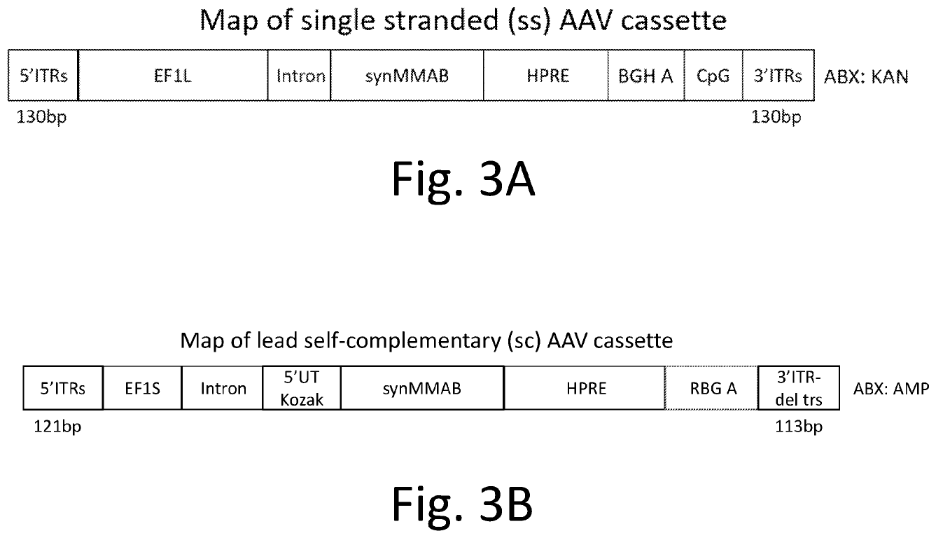

To examine function of the synMMAB polynucleotides, each gene was cloned into an AAV expression plasmid for the testing of MMAB protein after transfection into the MA/AB knockout cell line described in Example 1. The plasmid contained an AAV backbone designed to express an AAV transgene either in the single-strand configuration (FIG. 3A) or self-complementary configuration (FIG. 3B). The plasmid sequences are listed in Table 4. Table 5 summarizes the expression vectors studied.

TABLE 4
SEQ ID NO: 8
pAAV EF1L MMAB WT 6124 bp
CTTTCCTGCGTTATCCCCTGATTCTGTGGATAACCGTATTACCGCCTTTGAGTGAG
CTGATACCGCTCGCCGCAGCCGAACGACCGAGCGCAGCGAGTCAGTGAGCGAGG
AAGCGGAAGAGCGCCCAATACGCAAACCGCCTCTCCCCGCGCGTTGGCCGATTC
ATTAATGCAGCTGGCACGACAGGTTTCCCGACTGGAAAGCGGGCAGTGAGCGCA
ACGCAATTAATACGCGTACCGCTAGCCAGGAAGAGTTTGTAGAAACGCAAAAAG
GCCATCCGTCAGGATGGCCTTCTGCTTAGTTTGATGCCTGGCAGTTTATGGCGGG
CGTCCTGCCCGCCACCCTCCGGGCCGTTGCTTCACAACGTTCAAATCCGCTCCCG
GCGGATTTGTCCTACTCAGGAGAGCGTTCACCGACAAACAACAGATAAAACGAA
AGGCCCAGTCTTCCGACTGAGCCTTTCGTTTTATTTGATGCCTGGCAGTTCCCTAC
TCTCGCGTTAACGCTAGCATGGATGTTTTCCCAGTCACGACGTTGTAAAACGACG
GCCAGTCTTAAGCTCGGGCCCCAAATAATGATTTTATTTTGACTGATAGTGACCT
GTTCGTTGCAACAAATTGATGAGCAATGCTTTTTTATAATGCCAACTTTGTACAA
AAAAGCAGGCTTCTAGACTGCGCGCTCGCTCGCTCACTGAGGCCGCCCGGGCAA
AGCCCGGGCGTCGGGCGACCTTTGGTCGCCCGGCCTCAGTGAGCGAGCGAGCGC
GCAGAGAGGGAGTGGCCAACTCCATCACTAGGGGTTCCTTTAATTAATACGTAG
GTACCGGCTCCGGTGCCCGTCAGTGGGCAGAGCGCACATCGCCCACAGTCCCCG
AGAAGTTGGGGGGAGGGGTCGGCAATTGAACCGGTGCCTAGAGAAGGTGGCGC
GGGGTAAACTGGGAAAGTGATGTCGTGTACTGGCTCCGCCTTTTTCCCGAGGGTG
GGGGAGAACCGTATATAAGTGCAGTAGTCGCCGTGAACGTTCTTTTTCGCAACGG
GTTTGCCGCCAGAACACAGGTAAGTGCCGTGTGTGGTTCCCGCGGGCCTGGCCTC
TTTACGGGTTATGGCCCTTGCGTGCCTTGAATTACTTCCACCTGGCTGCAGTACGT
GATTCTTGATCCCGAGCTTCGGGTTGGAAGTGGGTGGGAGAGTTCGAGGCCTTGC
GCTTAAGGAGCCCCTTCGCCTCGTGCTTGAGTTGAGGCCTGGCCTGGGCGCTGGG
GCCGCCGCGTGCGAATCTGGTGGCACCTTCGCGCCTGTCTCGCTGCTTTCGATAA
GTCTCTAGCCATTTAAAATTTTTGATGACCTGCTGCGACGCTTTTTTTCTGGCAAG
ATAGTCTTGTAAATGCGGGCCAAGATCTGCACACTGGTATTTCGGTTTTTGGGGC
CGCGGGCGGCGACGGGGCCCGTGCGTCCCAGCGCACATGTTCGGCGAGGCGGGG
CCTGCGAGCGCGGCCACCGAGAATCGGACGGGGGTAGTCTCAAGCTGGCCGGCC
TGCTCTGGTGCCTGGCCTCGCGCCGCCGTGTATCGCCCCGCCCTGGGCGGCAAGG
CTGGCCCGGTCGGCACCAGTTGCGTGAGCGGAAAGATGGCCGCTTCCCGGCCCT
GCTGCAGGGAGCTCAAAATGGAGGACGCGGCGCTCGGGAGAGCGGGCGGGTGA
GTCACCCACACAAAGGAAAAGGGCCTTTCCGTCCTCAGCCGTCGCTTCATGTGAC
TCCACGGAGTACCGGGCGCCGTCCAGGCACCTCGATTAGTTCTCGAGCTTTTGGA
GTACGTCGTCTTTAGGTTGGGGGGAGGGGTTTTATGCGATGGAGTTTCCCCACAC
TGAGTGGGTGGAGACTGAAGTTAGGCCAGCTTGGCACTTGATGTAATTCTCCTTG
GAATTTGCCCTTTTTGAGTTTGGATCTTGGTTCATTCTCAAGCCTCAGACAGTGGT
TCAAAGTTTTTTTCTTCCATTTCAGGTGTCGTGAGCTAGAGCTTTATTGCGGTAGT
TTATCACAGTTAAATTGCTAACGCAGTCAGTGCTTCTGACACAACAGTCTCGAAC
TTAAGCTGCAGAAGTTGGTCGTGAGGCACTGGGCAGGTAAGTATCAAGGTTACA
AGACAGGTTTAAGGAGACCAATAGAAACTGGGCTTGTCGAGACAGAGAAGACTC
TTGCGTTTCTGATAGGCACCTATTGGTCTTACTGACATCCACTTTGCCTTTCTCTC
CACAGGTGTCCACTCCCAGTTCAATTACAGCTCTTAAGGCTAGAGTACTGAATTC
ATGGCTGTGTGCGGCCTGGGGAGCCGTCTTGGCCTGGGGAGCCGTCTTGGCCTGC
GCGGGTGCTTCGGCGCCGCCAGGCTCCTGTATCCCCGTTTCCAGAGCCGCGGCCC
TCAGGGCGTGGAAGACGGGGACAGGCCACAGCCTTCCTCGAAGACACCCAGGAT
CCCCAAGATTTACACCAAAACGGGAGACAAAGGGTTTTCTAGTACCTTCACAGG
AGAAAGGAGACCCAAAGATGACCAAGTGTTTGAAGCCGTGGGAACTACAGATGA
ATTAAGTTCAGCTATTGGGTTTGCTCTGGAATTAGTCACAGAAAAGGGCCATACA
TTTGCCGAAGAGCTTCAGAAAATCCAGTGCACATTGCAGGACGTCGGCTCGGCCC
TGGCGACACCATGCTCCTCGGCCCGGGAGGCTCACTTAAAGTATACCACGTTCAA
GGCGGGGCCCATCCTGGAGCTGGAGCAGTGGATCGACAAGTACACCAGCCAGCT
CCCACCACTCACGGCCTTCATCCTGCCTTCGGGAGGCAAGATCAGCTCGGCGCTG
CATTTCTGCCGGGCCGTGTGCCGCCGGGCCGAGAGACGTGTGGTGCCTCTTGTCC
AGATGGGAGAGACCGATGCGAACGTGGCCAAGTTCTTAAACAGACTCAGTGACT
ATCTCTTCACGCTAGCCAGATATGCAGCCATGAAGGAGGGGAATCAAGAGAAAA
TATACATGAAAAATGACCCATCGGCCGAGTCTGAGGGACTCTGAGGCCTCGAGA
TAACAGGCCTATTGATTGGAAAGTTTGTCAACGAATTGTGGGTCTTTTGGGGTTT
GCTGCCCCTTTTACGCAATGTGGATATCCTGCTTTAATGCCTTTATATGCATGTAT
ACAAGCAAAACAGGCTTTTACTTTCTCGCCAACTTACAAGGCCTTTCTCAGTAAA
CAGTATATGACCCTTTACCCCGTTGCTCGGCAACGGCCTGGTCTGTGCCAAGTGT
TTGCTGACGCAACCCCCACTGGTTGGGGCTTGGCCATAGGCCATCAGCGCATGCG
TGGAACCTTTGTGTCTCCTCTGCCGATCCATACTGCGGAACTCCTAGCCGCTTGTT
TTGCTCGCAGCAGGTCTGGAGCAAACCTCATCGGGACCGACAATTCTGTCGTACT
CTCCCGCAAGTATACATCGTTTCCATGGCTGCTAGGCTGTGCTGCCAACTGGATC
CTGCGCGGGACGTCCTTTGTTTACGTCCCGTCGGCGCTGAATCCCGCGGACGACC
CCTCCCGGGGCCGCTTGGGGCTCTACCGCCCGCTTCTCCGTCTGCCGTACCGTCC
GACCACGGGGCGCACCTCTCTTTACGCGGACTCCCCGTCTGTGCCTTCTCATCTG
CCGGACCGTGTGCACTTCGCTTCACCTCTGCACGTCGCATGGAGGCCACCGTGAA
CGCCCACCGGAACCTGCCCAAGGTCTTGCATAAGAGGACTCTTGGACTTTCAGCA
ATGTCATCGATATCGTCGACTCGCTGATCAGCCTCGACTGTGCCTTCTAGTTGCCA
GCCATCTGTTGTTTGCCCCTCCCCCGTGCCTTCCTTGACCCTGGAAGGTGCCACTC
CCACTGTCCTTTCCTAATAAAATGAGGAAATTGCATCGCATTGTCTGAGTAGGTG
TCATTCTATTCTGGGGGGTGGGGTGGGGCAGGACAGCAAGGGGGAGGATTGGGA
AGACAATAGCAGGCATGCTGGGGATGCGGTGGGCTCTATGGCTTCTGAGGCGGA
AAGAACCAGCTGGGGCTCGACTAGACTAGTCCTGCAGGTACGTAAGCGGCCGCG
GCCTAGGAACCCCTAGTGATGGAGTTGGCCACTCCCTCTCTGCGCGCTCGCTCGC
TCACTGAGGCCGGGCGACCAAAGGTCGCCCGACGCCCGGGCTTTGCCCGGGCGG
CCTCAGTGAGCGAGCGAGCGCGCAGCATATGACCCAGCTTTCTTGTACAAAGTTG
GCATTATAAGAAAGCATTGCTTATCAATTTGTTGCAACGAACAGGTCACTATCAG
TCAAAATAAAATCATTATTTGCCATCCAGCTGATATCCCCTATAGTGAGTCGTAT
TACATGGTCATAGCTGTTTCCTGGCAGCTCTGGCCCGTGTCTCAAAATCTCTGATG
TTACATTGCACAAGATAAAAATATATCATCATGAACAATAAAACTGTCTGCTTAC
ATAAACAGTAATACAAGGGGTGTTATGAGCCATATTCAACGGGAAACGTCGAGG
CCGCGATTAAATTCCAACATGGATGCTGATTTATATGGGTATAAATGGGCTCGCG
ATAATGTCGGGCAATCAGGTGCGACAATCTATCGCTTGTATGGGAAGCCCGATGC
GCCAGAGTTGTTTCTGAAACATGGCAAAGGTAGCGTTGCCAATGATGTTACAGAT
GAGATGGTCAGACTAAACTGGCTGACGGAATTTATGCCTCTTCCGACCATCAAGC
ATTTTATCCGTACTCCTGATGATGCATGGTTACTCACCACTGCGATCCCCGGAAA
AACAGCATTCCAGGTATTAGAAGAATATCCTGATTCAGGTGAAAATATTGTTGAT
GCGCTGGCAGTGTTCCTGCGCCGGTTGCATTCGATTCCTGTTTGTAATTGTCCTTT
TAACAGCGATCGCGTATTTCGTCTCGCTCAGGCGCAATCACGAATGAATAACGGT
TTGGTTGATGCGAGTGATTTTGATGACGAGCGTAATGGCTGGCCTGTTGAACAAG
TCTGGAAAGAAATGCATAAACTTTTGCCATTCTCACCGGATTCAGTCGTCACTCA
TGGTGATTTCTCACTTGATAACCTTATTTTTGACGAGGGGAAATTAATAGGTTGT
ATTGATGTTGGACGAGTCGGAATCGCAGACCGATACCAGGATCTTGCCATCCTAT
GGAACTGCCTCGGTGAGTTTTCTCCTTCATTACAGAAACGGCTTTTTCAAAAATA
TGGTATTGATAATCCTGATATGAATAAATTGCAGTTTCATTTGATGCTCGATGAG
TTTTTCTAATCAGAATTGGTTAATTGGTTGTAACACTGGCAGAGCATTACGCTGA
CTTGACGGGACGGCGCAAGCTCATGACCAAAATCCCTTAACGTGAGTTACGCGTC
GTTCCACTGAGCGTCAGACCCCGTAGAAAAGATCAAAGGATCTTCTTGAGATCCT
TTTTTTCTGCGCGTAATCTGCTGCTTGCAAACAAAAAAACCACCGCTACCAGCGG
TGGTTTGTTTGCCGGATCAAGAGCTACCAACTCTTTTTCCGAAGGTAACTGGCTTC
AGCAGAGCGCAGATACCAAATACTGTTCTTCTAGTGTAGCCGTAGTTAGGCCACC
ACTTCAAGAACTCTGTAGCACCGCCTACATACCTCGCTCTGCTAATCCTGTTACC
AGTGGCTGCTGCCAGTGGCGATAAGTCGTGTCTTACCGGGTTGGACTCAAGACGA
TAGTTACCGGATAAGGCGCAGCGGTCGGGCTGAACGGGGGGTTCGTGCACACAG
CCCAGCTTGGAGCGAACGACCTACACCGAACTGAGATACCTACAGCGTGAGCTA
TGAGAAAGCGCCACGCTTCCCGAAGGGAGAAAGGCGGACAGGTATCCGGTAAGC
GGCAGGGTCGGAACAGGAGAGCGCACGAGGGAGCTTCCAGGGGGAAACGCCTG
GTATCTTTATAGTCCTGTCGGGTTTCGCCACCTCTGACTTGAGCGTCGATTTTTGT
GATGCTCGTCAGGGGGGCGGAGCCTATGGAAAAACGCCAGCAACGCGGCCTTTT
TACGGTTCCTGGCCTTTTGCTGGCCTTTTGCTCACATGTT
SEQ ID NO: 9
pAAV EF1L synMMAB1 6112 bp
CTTTCCTGCGTTATCCCCTGATTCTGTGGATAACCGTATTACCGCCTTTGAGTGAG
CTGATACCGCTCGCCGCAGCCGAACGACCGAGCGCAGCGAGTCAGTGAGCGAGG
AAGCGGAAGAGCGCCCAATACGCAAACCGCCTCTCCCCGCGCGTTGGCCGATTC
ATTAATGCAGCTGGCACGACAGGTTTCCCGACTGGAAAGCGGGCAGTGAGCGCA
ACGCAATTAATACGCGTACCGCTAGCCAGGAAGAGTTTGTAGAAACGCAAAAAG
GCCATCCGTCAGGATGGCCTTCTGCTTAGTTTGATGCCTGGCAGTTTATGGCGGG
CGTCCTGCCCGCCACCCTCCGGGCCGTTGCTTCACAACGTTCAAATCCGCTCCCG
GCGGATTTGTCCTACTCAGGAGAGCGTTCACCGACAAACAACAGATAAAACGAA
AGGCCCAGTCTTCCGACTGAGCCTTTCGTTTTATTTGATGCCTGGCAGTTCCCTAC
TCTCGCGTTAACGCTAGCATGGATGTTTTCCCAGTCACGACGTTGTAAAACGACG
GCCAGTCTTAAGCTCGGGCCCCAAATAATGATTTTATTTTGACTGATAGTGACCT
GTTCGTTGCAACAAATTGATGAGCAATGCTTTTTTATAATGCCAACTTTGTACAA
AAAAGCTGGGTCATATGCTGCGCGCTCGCTCGCTCACTGAGGCCGCCCGGGCAA
AGCCCGGGCGTCGGGCGACCTTTGGTCGCCCGGCCTCAGTGAGCGAGCGAGCGC
GCAGAGAGGGAGTGGCCAACTCCATCACTAGGGGTTCCTTTAATTAAGGCTCCG
GTGCCCGTCAGTGGGCAGAGCGCACATCGCCCACAGTCCCCGAGAAGTTGGGGG
GAGGGGTCGGCAATTGAACCGGTGCCTAGAGAAGGTGGCGCGGGGTAAACTGGG
AAAGTGATGTCGTGTACTGGCTCCGCCTTTTTCCCGAGGGTGGGGGAGAACCGTA
TATAAGTGCAGTAGTCGCCGTGAACGTTCTTTTTCGCAACGGGTTTGCCGCCAGA
ACACAGGTAAGTGCCGTGTGTGGTTCCCGCGGGCCTGGCCTCTTTACGGGTTATG
GCCCTTGCGTGCCTTGAATTACTTCCACCTGGCTGCAGTACGTGATTCTTGATCCC
GAGCTTCGGGTTGGAAGTGGGTGGGAGAGTTCGAGGCCTTGCGCTTAAGGAGCC
CCTTCGCCTCGTGCTTGAGTTGAGGCCTGGCCTGGGCGCTGGGGCCGCCGCGTGC
GAATCTGGTGGCACCTTCGCGCCTGTCTCGCTGCTTTCGATAAGTCTCTAGCCATT
TAAAATTTTTGATGACCTGCTGCGACGCTTTTTTTCTGGCAAGATAGTCTTGTAAA
TGCGGGCCAAGATCTGCACACTGGTATTTCGGTTTTTGGGGCCGCGGGCGGCGAC
GGGGCCCGTGCGTCCCAGCGCACATGTTCGGCGAGGCGGGGCCTGCGAGCGCGG
CCACCGAGAATCGGACGGGGGTAGTCTCAAGCTGGCCGGCCTGCTCTGGTGCCT
GGCCTCGCGCCGCCGTGTATCGCCCCGCCCTGGGCGGCAAGGCTGGCCCGGTCG
GCACCAGTTGCGTGAGCGGAAAGATGGCCGCTTCCCGGCCCTGCTGCAGGGAGC
TCAAAATGGAGGACGCGGCGCTCGGGAGAGCGGGCGGGTGAGTCACCCACACA
AAGGAAAAGGGCCTTTCCGTCCTCAGCCGTCGCTTCATGTGACTCCACGGAGTAC
CGGGCGCCGTCCAGGCACCTCGATTAGTTCTCGAGCTTTTGGAGTACGTCGTCTT
TAGGTTGGGGGGAGGGGTTTTATGCGATGGAGTTTCCCCACACTGAGTGGGTGG
AGACTGAAGTTAGGCCAGCTTGGCACTTGATGTAATTCTCCTTGGAATTTGCCCT
TTTTGAGTTTGGATCTTGGTTCATTCTCAAGCCTCAGACAGTGGTTCAAAGTTTTT
TTCTTCCATTTCAGGTGTCGTGAGCTAGAGCTTTATTGCGGTAGTTTATCACAGTT
AAATTGCTAACGCAGTCAGTGCTTCTGACACAACAGTCTCGAACTTAAGCTGCAG
AAGTTGGTCGTGAGGCACTGGGCAGGTAAGTATCAAGGTTACAAGACAGGTTTA
AGGAGACCAATAGAAACTGGGCTTGTCGAGACAGAGAAGACTCTTGCGTTTCTG
ATAGGCACCTATTGGTCTTACTGACATCCACTTTGCCTTTCTCTCCACAGGTGTCC
ACTCCCAGTTCAATTACAGCTCTTAAGGCTAGAGTACTGAATTCATGGCTGTCTG
CGGTCTGGGCAGCAGACTGGGTCTTGGGTCCCGCTTGGGACTCCGAGGCTGCTTT
GGTGCAGCACGCTTGCTGTATCCTCGCTTCCAGTCTAGAGGGCCACAAGGAGTAG
AGGACGGTGATCGGCCCCAGCCAAGCAGCAAAACACCGAGGATACCGAAAATTT
ATACAAAGACGGGAGATAAGGGGTTTAGCAGCACCTTCACGGGCGAGCGACGGC
CCAAGGACGACCAAGTATTCGAGGCAGTCGGGACGACGGACGAACTGTCAAGTG
CTATTGGGTTCGCTCTGGAGCTGGTTACGGAGAAGGGACATACTTTCGCGGAGGA
GCTCCAAAAAATACAATGCACCCTTCAAGACGTGGGCAGTGCCCTGGCGACACC
GTGTAGCAGCGCGCGCGAGGCCCACTTGAAATATACAACTTTCAAAGCAGGACC
TATATTGGAACTCGAGCAGTGGATAGACAAATACACGTCCCAACTGCCTCCCCTG
ACAGCTTTTATCTTGCCATCTGGCGGTAAGATAAGTTCTGCTCTTCATTTCTGTAG
GGCAGTATGTAGGCGAGCGGAAAGGCGGGTTGTACCCCTGGTGCAGATGGGTGA
GACTGACGCCAATGTGGCCAAGTTCTTGAACCGCCTGTCTGATTATCTGTTTACA
CTCGCTAGATACGCCGCTATGAAAGAAGGAAATCAAGAGAAGATATACATGAAG
AACGATCCGTCAGCGGAATCCGAGGGGCTCTGAGGCCTCGAGATAACAGGCCTA
TTGATTGGAAAGTTTGTCAACGAATTGTGGGTCTTTTGGGGTTTGCTGCCCCTTTT
ACGCAATGTGGATATCCTGCTTTAATGCCTTTATATGCATGTATACAAGCAAAAC
AGGCTTTTACTTTCTCGCCAACTTACAAGGCCTTTCTCAGTAAACAGTATATGACC
CTTTACCCCGTTGCTCGGCAACGGCCTGGTCTGTGCCAAGTGTTTGCTGACGCAA
CCCCCACTGGTTGGGGCTTGGCCATAGGCCATCAGCGCATGCGTGGAACCTTTGT
GTCTCCTCTGCCGATCCATACTGCGGAACTCCTAGCCGCTTGTTTTGCTCGCAGCA
GGTCTGGAGCAAACCTCATCGGGACCGACAATTCTGTCGTACTCTCCCGCAAGTA
TACATCGTTTCCATGGCTGCTAGGCTGTGCTGCCAACTGGATCCTGCGCGGGACG
TCCTTTGTTTACGTCCCGTCGGCGCTGAATCCCGCGGACGACCCCTCCCGGGGCC
GCTTGGGGCTCTACCGCCCGCTTCTCCGTCTGCCGTACCGTCCGACCACGGGGCG
CACCTCTCTTTACGCGGACTCCCCGTCTGTGCCTTCTCATCTGCCGGACCGTGTGC
ACTTCGCTTCACCTCTGCACGTCGCATGGAGGCCACCGTGAACGCCCACCGGAAC
CTGCCCAAGGTCTTGCATAAGAGGACTCTTGGACTTTCAGCAATGTCATCGATAT
CGTCGACTCGCTGATCAGCCTCGACTGTGCCTTCTAGTTGCCAGCCATCTGTTGTT
TGCCCCTCCCCCGTGCCTTCCTTGACCCTGGAAGGTGCCACTCCCACTGTCCTTTC
CTAATAAAATGAGGAAATTGCATCGCATTGTCTGAGTAGGTGTCATTCTATTCTG
GGGGGTGGGGTGGGGCAGGACAGCAAGGGGGAGGATTGGGAAGACAATAGCAG
GCATGCTGGGGATGCGGTGGGCTCTATGGCTTCTGAGGCGGAAAGAACCAGCTG
GGGCTCGACTAGACTAGTCCTGCAGGTACGTAAGCGGCCGCGGCCTAGGAACCC
CTAGTGATGGAGTTGGCCACTCCCTCTCTGCGCGCTCGCTCGCTCACTGAGGCCG
GGCGACCAAAGGTCGCCCGACGCCCGGGCTTTGCCCGGGCGGCCTCAGTGAGCG
AGCGAGCGCGCAGCATATGACCCAGCTTTCTTGTACAAAGTTGGCATTATAAGAA
AGCATTGCTTATCAATTTGTTGCAACGAACAGGTCACTATCAGTCAAAATAAAAT
CATTATTTGCCATCCAGCTGATATCCCCTATAGTGAGTCGTATTACATGGTCATAG
CTGTTTCCTGGCAGCTCTGGCCCGTGTCTCAAAATCTCTGATGTTACATTGCACAA
GATAAAAATATATCATCATGAACAATAAAACTGTCTGCTTACATAAACAGTAATA
CAAGGGGTGTTATGAGCCATATTCAACGGGAAACGTCGAGGCCGCGATTAAATT
CCAACATGGATGCTGATTTATATGGGTATAAATGGGCTCGCGATAATGTCGGGCA
ATCAGGTGCGACAATCTATCGCTTGTATGGGAAGCCCGATGCGCCAGAGTTGTTT
CTGAAACATGGCAAAGGTAGCGTTGCCAATGATGTTACAGATGAGATGGTCAGA
CTAAACTGGCTGACGGAATTTATGCCTCTTCCGACCATCAAGCATTTTATCCGTA
CTCCTGATGATGCATGGTTACTCACCACTGCGATCCCCGGAAAAACAGCATTCCA
GGTATTAGAAGAATATCCTGATTCAGGTGAAAATATTGTTGATGCGCTGGCAGTG
TTCCTGCGCCGGTTGCATTCGATTCCTGTTTGTAATTGTCCTTTTAACAGCGATCG
CGTATTTCGTCTCGCTCAGGCGCAATCACGAATGAATAACGGTTTGGTTGATGCG
AGTGATTTTGATGACGAGCGTAATGGCTGGCCTGTTGAACAAGTCTGGAAAGAA
ATGCATAAACTTTTGCCATTCTCACCGGATTCAGTCGTCACTCATGGTGATTTCTC
ACTTGATAACCTTATTTTTGACGAGGGGAAATTAATAGGTTGTATTGATGTTGGA
CGAGTCGGAATCGCAGACCGATACCAGGATCTTGCCATCCTATGGAACTGCCTCG
GTGAGTTTTCTCCTTCATTACAGAAACGGCTTTTTCAAAAATATGGTATTGATAAT
CCTGATATGAATAAATTGCAGTTTCATTTGATGCTCGATGAGTTTTTCTAATCAGA
ATTGGTTAATTGGTTGTAACACTGGCAGAGCATTACGCTGACTTGACGGGACGGC
GCAAGCTCATGACCAAAATCCCTTAACGTGAGTTACGCGTCGTTCCACTGAGCGT
CAGACCCCGTAGAAAAGATCAAAGGATCTTCTTGAGATCCTTTTTTTCTGCGCGT
AATCTGCTGCTTGCAAACAAAAAAACCACCGCTACCAGCGGTGGTTTGTTTGCCG
GATCAAGAGCTACCAACTCTTTTTCCGAAGGTAACTGGCTTCAGCAGAGCGCAGA
TACCAAATACTGTTCTTCTAGTGTAGCCGTAGTTAGGCCACCACTTCAAGAACTC
TGTAGCACCGCCTACATACCTCGCTCTGCTAATCCTGTTACCAGTGGCTGCTGCC
AGTGGCGATAAGTCGTGTCTTACCGGGTTGGACTCAAGACGATAGTTACCGGATA
AGGCGCAGCGGTCGGGCTGAACGGGGGGTTCGTGCACACAGCCCAGCTTGGAGC
GAACGACCTACACCGAACTGAGATACCTACAGCGTGAGCTATGAGAAAGCGCCA
CGCTTCCCGAAGGGAGAAAGGCGGACAGGTATCCGGTAAGCGGCAGGGTCGGA
ACAGGAGAGCGCACGAGGGAGCTTCCAGGGGGAAACGCCTGGTATCTTTATAGT
CCTGTCGGGTTTCGCCACCTCTGACTTGAGCGTCGATTTTTGTGATGCTCGTCAGG
GGGGCGGAGCCTATGGAAAAACGCCAGCAACGCGGCCTTTTTACGGTTCCTGGC
CTTTTGCTGGCCTTTTGCTCACATGTT
SEQ ID NO: 10
pAAV EF1L synMMAB2 6112
CTTTCCTGCGTTATCCCCTGATTCTGTGGATAACCGTATTACCGCCTTTGAGTGAG
CTGATACCGCTCGCCGCAGCCGAACGACCGAGCGCAGCGAGTCAGTGAGCGAGG
AAGCGGAAGAGCGCCCAATACGCAAACCGCCTCTCCCCGCGCGTTGGCCGATTC
ATTAATGCAGCTGGCACGACAGGTTTCCCGACTGGAAAGCGGGCAGTGAGCGCA
ACGCAATTAATACGCGTACCGCTAGCCAGGAAGAGTTTGTAGAAACGCAAAAAG
GCCATCCGTCAGGATGGCCTTCTGCTTAGTTTGATGCCTGGCAGTTTATGGCGGG
CGTCCTGCCCGCCACCCTCCGGGCCGTTGCTTCACAACGTTCAAATCCGCTCCCG
GCGGATTTGTCCTACTCAGGAGAGCGTTCACCGACAAACAACAGATAAAACGAA
AGGCCCAGTCTTCCGACTGAGCCTTTCGTTTTATTTGATGCCTGGCAGTTCCCTAC
TCTCGCGTTAACGCTAGCATGGATGTTTTCCCAGTCACGACGTTGTAAAACGACG
GCCAGTCTTAAGCTCGGGCCCCAAATAATGATTTTATTTTGACTGATAGTGACCT
GTTCGTTGCAACAAATTGATGAGCAATGCTTTTTTATAATGCCAACTTTGTACAA
AAAAGCAGGCTTCTAGACTGCGCGCTCGCTCGCTCACTGAGGCCGCCCGGGCAA
AGCCCGGGCGTCGGGCGACCTTTGGTCGCCCGGCCTCAGTGAGCGAGCGAGCGC
GCAGAGAGGGAGTGGCCAACTCCATCACTAGGGGTTCCTTTAATTAAGGCTCCG
GTGCCCGTCAGTGGGCAGAGCGCACATCGCCCACAGTCCCCGAGAAGTTGGGGG
GAGGGGTCGGCAATTGAACCGGTGCCTAGAGAAGGTGGCGCGGGGTAAACTGGG
AAAGTGATGTCGTGTACTGGCTCCGCCTTTTTCCCGAGGGTGGGGGAGAACCGTA
TATAAGTGCAGTAGTCGCCGTGAACGTTCTTTTTCGCAACGGGTTTGCCGCCAGA
ACACAGGTAAGTGCCGTGTGTGGTTCCCGCGGGCCTGGCCTCTTTACGGGTTATG
GCCCTTGCGTGCCTTGAATTACTTCCACCTGGCTGCAGTACGTGATTCTTGATCCC
GAGCTTCGGGTTGGAAGTGGGTGGGAGAGTTCGAGGCCTTGCGCTTAAGGAGCC
CCTTCGCCTCGTGCTTGAGTTGAGGCCTGGCCTGGGCGCTGGGGCCGCCGCGTGC
GAATCTGGTGGCACCTTCGCGCCTGTCTCGCTGCTTTCGATAAGTCTCTAGCCATT
TAAAATTTTTGATGACCTGCTGCGACGCTTTTTTTCTGGCAAGATAGTCTTGTAAA
TGCGGGCCAAGATCTGCACACTGGTATTTCGGTTTTTGGGGCCGCGGGCGGCGAC
GGGGCCCGTGCGTCCCAGCGCACATGTTCGGCGAGGCGGGGCCTGCGAGCGCGG
CCACCGAGAATCGGACGGGGGTAGTCTCAAGCTGGCCGGCCTGCTCTGGTGCCT
GGCCTCGCGCCGCCGTGTATCGCCCCGCCCTGGGCGGCAAGGCTGGCCCGGTCG
GCACCAGTTGCGTGAGCGGAAAGATGGCCGCTTCCCGGCCCTGCTGCAGGGAGC
TCAAAATGGAGGACGCGGCGCTCGGGAGAGCGGGGGGTGAGTCACCCACACA
AAGGAAAAGGGCCTTTCCGTCCTCAGCCGTCGCTTCATGTGACTCCACGGAGTAC
CGGGCGCCGTCCAGGCACCTCGATTAGTTCTCGAGCTTTTGGAGTACGTCGTCTT
TAGGTTGGGGGGAGGGGTTTTATGCGATGGAGTTTCCCCACACTGAGTGGGTGG
AGACTGAAGTTAGGCCAGCTTGGCACTTGATGTAATTCTCCTTGGAATTTGCCCT
TTTTGAGTTTGGATCTTGGTTCATTCTCAAGCCTCAGACAGTGGTTCAAAGTTTTT
TTCTTCCATTTCAGGTGTCGTGAGCTAGAGCTTTATTGCGGTAGTTTATCACAGTT
AAATTGCTAACGCAGTCAGTGCTTCTGACACAACAGTCTCGAACTTAAGCTGCAG
AAGTTGGTCGTGAGGCACTGGGCAGGTAAGTATCAAGGTTACAAGACAGGTTTA
AGGAGACCAATAGAAACTGGGCTTGTCGAGACAGAGAAGACTCTTGCGTTTCTG
ATAGGCACCTATTGGTCTTACTGACATCCACTTTGCCTTTCTCTCCACAGGTGTCC
ACTCCCAGTTCAATTACAGCTCTTAAGGCTAGAGTACTGAATTCATGGCGGTCTG
CGGATTGGGTTCACGATTGGGCCTTGGGTCTAGGCTCGGCCTGCGAGGTTGTTTC
GGAGCTGCTAGATTGTTGTACCCACGATTCCAAAGCAGGGGACCACAAGGTGTG
GAAGACGGTGACAGGCCTCAGCCCTCAAGCAAAACCCCTCGGATTCCGAAAATA
TATACAAAGACAGGAGACAAAGGCTTTTCAAGCACGTTTACTGGCGAACGAAGG
CCTAAAGACGATCAAGTTTTTGAGGCGGTAGGGACAACTGACGAACTTTCATCTG
CGATAGGCTTCGCGCTCGAGTTGGTGACGGAGAAGGGTCATACATTCGCTGAAG
AACTTCAGAAAATTCAGTGTACGCTCCAAGATGTTGGCAGTGCGCTTGCGACACC
TTGCAGCTCAGCAAGGGAAGCGCATTTGAAGTACACCACTTTCAAAGCGGGCCC
GATTCTCGAACTTGAACAATGGATTGACAAATATACAAGCCAACTGCCGCCTCTT
ACCGCCTTCATCCTCCCTAGTGGTGGCAAAATTTCTTCCGCGCTTCATTTTTGTAG
GGCCGTGTGTCGCCGCGCCGAGAGACGGGTGGTTCCGTTGGTACAGATGGGTGA
AACAGATGCAAACGTGGCAAAGTTCCTTAACCGCCTCAGCGACTACTTGTTCACT
CTCGCTAGGTATGCCGCCATGAAAGAAGGGAATCAAGAGAAGATCTATATGAAG
AATGACCCGAGCGCTGAAAGCGAGGGACTTTGAGGCCTCGAGATAACAGGCCTA
TTGATTGGAAAGTTTGTCAACGAATTGTGGGTCTTTTGGGGTTTGCTGCCCCTTTT
ACGCAATGTGGATATCCTGCTTTAATGCCTTTATATGCATGTATACAAGCAAAAC
AGGCTTTTACTTTCTCGCCAACTTACAAGGCCTTTCTCAGTAAACAGTATATGACC
CTTTACCCCGTTGCTCGGCAACGGCCTGGTCTGTGCCAAGTGTTTGCTGACGCAA
CCCCCACTGGTTGGGGCTTGGCCATAGGCCATCAGCGCATGCGTGGAACCTTTGT
GTCTCCTCTGCCGATCCATACTGCGGAACTCCTAGCCGCTTGTTTTGCTCGCAGCA
GGTCTGGAGCAAACCTCATCGGGACCGACAATTCTGTCGTACTCTCCCGCAAGTA
TACATCGTTTCCATGGCTGCTAGGCTGTGCTGCCAACTGGATCCTGCGCGGGACG
TCCTTTGTTTACGTCCCGTCGGCGCTGAATCCCGCGGACGACCCCTCCCGGGGCC
GCTTGGGGCTCTACCGCCCGCTTCTCCGTCTGCCGTACCGTCCGACCACGGGGCG
CACCTCTCTTTACGCGGACTCCCCGTCTGTGCCTTCTCATCTGCCGGACCGTGTGC
ACTTCGCTTCACCTCTGCACGTCGCATGGAGGCCACCGTGAACGCCCACCGGAAC
CTGCCCAAGGTCTTGCATAAGAGGACTCTTGGACTTTCAGCAATGTCATCGATAT
CGTCGACTCGCTGATCAGCCTCGACTGTGCCTTCTAGTTGCCAGCCATCTGTTGTT
TGCCCCTCCCCCGTGCCTTCCTTGACCCTGGAAGGTGCCACTCCCACTGTCCTTTC
CTAATAAAATGAGGAAATTGCATCGCATTGTCTGAGTAGGTGTCATTCTATTCTG
GGGGGTGGGGTGGGGCAGGACAGCAAGGGGGAGGATTGGGAAGACAATAGCAG
GCATGCTGGGGATGCGGTGGGCTCTATGGCTTCTGAGGCGGAAAGAACCAGCTG
GGGCTCGACTAGACTAGTCCTGCAGGTACGTAAGCGGCCGCGGCCTAGGAACCC
CTAGTGATGGAGTTGGCCACTCCCTCTCTGCGCGCTCGCTCGCTCACTGAGGCCG
GGCGACCAAAGGTCGCCCGACGCCCGGGCTTTGCCCGGGCGGCCTCAGTGAGCG
AGCGAGCGCGCAGCATATGACCCAGCTTTCTTGTACAAAGTTGGCATTATAAGAA
AGCATTGCTTATCAATTTGTTGCAACGAACAGGTCACTATCAGTCAAAATAAAAT
CATTATTTGCCATCCAGCTGATATCCCCTATAGTGAGTCGTATTACATGGTCATAG
CTGTTTCCTGGCAGCTCTGGCCCGTGTCTCAAAATCTCTGATGTTACATTGCACAA
GATAAAAATATATCATCATGAACAATAAAACTGTCTGCTTACATAAACAGTAATA
CAAGGGGTGTTATGAGCCATATTCAACGGGAAACGTCGAGGCCGCGATTAAATT
CCAACATGGATGCTGATTTATATGGGTATAAATGGGCTCGCGATAATGTCGGGCA
ATCAGGTGCGACAATCTATCGCTTGTATGGGAAGCCCGATGCGCCAGAGTTGTTT
CTGAAACATGGCAAAGGTAGCGTTGCCAATGATGTTACAGATGAGATGGTCAGA
CTAAACTGGCTGACGGAATTTATGCCTCTTCCGACCATCAAGCATTTTATCCGTA
CTCCTGATGATGCATGGTTACTCACCACTGCGATCCCCGGAAAAACAGCATTCCA
GGTATTAGAAGAATATCCTGATTCAGGTGAAAATATTGTTGATGCGCTGGCAGTG
TTCCTGCGCCGGTTGCATTCGATTCCTGTTTGTAATTGTCCTTTTAACAGCGATCG
CGTATTTCGTCTCGCTCAGGCGCAATCACGAATGAATAACGGTTTGGTTGATGCG
AGTGATTTTGATGACGAGCGTAATGGCTGGCCTGTTGAACAAGTCTGGAAAGAA
ATGCATAAACTTTTGCCATTCTCACCGGATTCAGTCGTCACTCATGGTGATTTCTC
ACTTGATAACCTTATTTTTGACGAGGGGAAATTAATAGGTTGTATTGATGTTGGA
CGAGTCGGAATCGCAGACCGATACCAGGATCTTGCCATCCTATGGAACTGCCTCG
GTGAGTTTTCTCCTTCATTACAGAAACGGCTTTTTCAAAAATATGGTATTGATAAT
CCTGATATGAATAAATTGCAGTTTCATTTGATGCTCGATGAGTTTTTCTAATCAGA
ATTGGTTAATTGGTTGTAACACTGGCAGAGCATTACGCTGACTTGACGGGACGGC
GCAAGCTCATGACCAAAATCCCTTAACGTGAGTTACGCGTCGTTCCACTGAGCGT
CAGACCCCGTAGAAAAGATCAAAGGATCTTCTTGAGATCCTTTTTTTCTGCGCGT
AATCTGCTGCTTGCAAACAAAAAAACCACCGCTACCAGCGGTGGTTTGTTTGCCG
GATCAAGAGCTACCAACTCTTTTTCCGAAGGTAACTGGCTTCAGCAGAGCGCAGA
TACCAAATACTGTTCTTCTAGTGTAGCCGTAGTTAGGCCACCACTTCAAGAACTC
TGTAGCACCGCCTACATACCTCGCTCTGCTAATCCTGTTACCAGTGGCTGCTGCC
AGTGGCGATAAGTCGTGTCTTACCGGGTTGGACTCAAGACGATAGTTACCGGATA
AGGCGCAGCGGTCGGGCTGAACGGGGGGTTCGTGCACACAGCCCAGCTTGGAGC
GAACGACCTACACCGAACTGAGATACCTACAGCGTGAGCTATGAGAAAGCGCCA
CGCTTCCCGAAGGGAGAAAGGCGGACAGGTATCCGGTAAGCGGCAGGGTCGGA
ACAGGAGAGCGCACGAGGGAGCTTCCAGGGGGAAACGCCTGGTATCTTTATAGT
CCTGTCGGGTTTCGCCACCTCTGACTTGAGCGTCGATTTTTGTGATGCTCGTCAGG
GGGGCGGAGCCTATGGAAAAACGCCAGCAACGCGGCCTTTTTACGGTTCCTGGC
CTTTTGCTGGCCTTTTGCTCACATGTT
SEQ ID NO: 11
pscAAV EF1s MMAB WT BGH 5216
GGCCACTCCCTCTCTGCGCGCTCGCTCGCTCACTGAGGCCGGGCGACCAAAGGTC
GCCCGGCCTCAGTGAGCGAGCGAGCGCGCAGAGAGGGAGTGGCCAACTCCATCA
CTAGGGGTTCCTGGAGGGGTGGAGTCGTGACCTAGGGGCTCCGGTGCCCGTCAG
TGGGCAGAGCGCACATCGCCCACAGTCCCCGAGAAGTTGGGGGGAGGGGTCGGC
AATTGAACCGGTGCCTAGAGAAGGTGGCGCGGGGTAAACTGGGAAAGTGATGTC
GTGTACTGGCTCCGCCTTTTTCCCGAGGGTGGGGGAGAACCGTATATAAGTGCAG
TAGTCGCCGTGAACGTTCTTTTTCGCAACGGGTTTGCCGCCAGAACACAGGTCAG
ATCAGATCTTTGTCGATCCTACCATCCACTCGACACACCCGCCAGCGGCCGCGTT
GGTATCAAGGTTACAAGACAGGTTTAAGGAGACCAATAGAAACTGGGCATGTGG
AGACAGAGAAGACTCTTGGGTTTCTGATAGGCACTGACTCTCTTCCTTTGTCCTGT
TCCCATTTCAGAAGCTTCCGAGCTCTCGAATTAAATAAGAGAGAAAAGAAGAGT
AAGAAGAAATATAAGAGCCACCATGGCTGTGTGCGGCCTGGGGAGCCGTCTTGG
CCTGGGGAGCCGTCTTGGCCTGCGCGGGTGCTTCGGCGCCGCCAGGCTCCTGTAT
CCCCGTTTCCAGAGCCGCGGCCCTCAGGGCGTGGAAGACGGGGACAGGCCACAG
CCTTCCTCGAAGACACCCAGGATCCCCAAGATTTACACCAAAACGGGAGACAAA
GGGTTTTCTAGTACCTTCACAGGAGAAAGGAGACCCAAAGATGACCAAGTGTTT
GAAGCCGTGGGAACTACAGATGAATTAAGTTCAGCTATTGGGTTTGCTCTGGAAT
TAGTCACAGAAAAGGGCCATACATTTGCCGAAGAGCTTCAGAAAATCCAGTGCA
CATTGCAGGACGTCGGCTCGGCCCTGGCGACACCATGCTCCTCGGCCCGGGAGG
CTCACTTAAAGTATACCACGTTCAAGGCGGGGCCCATCCTGGAGCTGGAGCAGT
GGATCGACAAGTACACCAGCCAGCTCCCACCACTCACGGCCTTCATCCTGCCTTC
GGGAGGCAAGATCAGCTCGGCGCTGCATTTCTGCCGGGCCGTGTGCCGCCGGGC
CGAGAGACGTGTGGTGCCTCTTGTCCAGATGGGAGAGACCGATGCGAACGTGGC
CAAGTTCTTAAACAGACTCAGTGACTATCTCTTCACGCTAGCCAGATATGCAGCC
ATGAAGGAGGGGAATCAAGAGAAAATATACATGAAAAATGACCCATCGGCCGA
GTCTGAGGGACTCTGAACTCGAGATAACAGGCCTATTGATTGGAAAGTTTGTCAA
CGAATTGTGGGTCTTTTGGGGTTTGCTGCCCCTTTTACGCAATGTGGATATCCTGC
TTTAATGCCTTTATATGCATGTATACAAGCAAAACAGGCTTTTACTTTCTCGCCAA
CTTACAAGGCCTTTCTCAGTAAACAGTATATGACCCTTTACCCCGTTGCTCGGCA
ACGGCCTGGTCTGTGCCAAGTGTTTGCTGACGCAACCCCCACTGGTTGGGGCTTG
GCCATAGGCCATCAGCGCATGCGTGGAACCTTTGTGTCTCCTCTGCCGATCCATA
CTGCGGAACTCCTAGCCGCTTGTTTTGCTCGCAGCAGGTCTGGAGCAAACCTCAT
CGGGACCGACAATTCTGTCGTACTCTCCCGCAAGTATACATCGTTTCCATGGCTG
CTAGGCTGTGCTGCCAACTGGATCCTGCGCGGGACGTCCTTTGTTTACGTCCCGT
CGGCGCTGAATCCCGCGGACGACCCCTCCCGGGGCCGCTTGGGGCTCTACCGCCC
GCTTCTCCGTCTGCCGTACCGTCCGACCACGGGGCGCACCTCTCTTTACGCGGAC
TCCCCGTCTGTGCCTTCTCATCTGCCGGACCGTGTGCACTTCGCTTCACCTCTGCA
CGTCGCATGGAGGCCACCGTGAACGCCCACCGGAACCTGCCCAAGGTCTTGCAT
AAGAGGACTCTTGGACTTTCAGCAATGTCATCGATATCGTCGACTCGCTGATCAG
CCTCGACTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGAGGAAA
TTGCATCGCATTGTCTGAGTAGGTGTCATTCTATTCTGGGGGGTGGGGGGGGCA
GGACAGCAAGGGGGAGGATTGGGAAGACAATAGCAGGCATGCTGGGGATGCGG
TGGGCTCTATGGCTTCTGAGGCGGAAAGAACCAGCTGGGGCTCGACTAGAACTA
GTCCACTCCCTCTCTGCGCGCTCGCTCGCTCACTGAGGCCGGGCGACCAAAGGTC
GCCCGACGCCCGGGCTTTGCCCGGGCGGCCTCAGTGAGCGAGCGAGCGCGCAGA
GAGGGACAGATCCGGGCCCGCATGCGTCGACAATTCACTGGCCGTCGTTTTACAA
CGTCGTGACTGGGAAAACCCTGGCGTTACCCAACTTAATCGCCTTGCAGCACATC
CCCCTTTCGCCAGCTGGCGTAATAGCGAAGAGGCCCGCACCGATCGCCCTTCCCA
ACAGTTGCGCAGCCTGAATGGCGAATGGCGCCTGATGCGGTATTTTCTCCTTACG
CATCTGTGCGGTATTTCACACCGCATATGGTGCACTCTCAGTACAATCTGCTCTG
ATGCCGCATAGTTAAGCCAGCCCCGACACCCGCCAACACCCGCTGACGCGCCCT
GACGGGCTTGTCTGCTCCCGGCATCCGCTTACAGACAAGCTGTGACCGTCTCCGG
GAGCTGCATGTGTCAGAGGTTTTCACCGTCATCACCGAAACGCGCGAGACGAAA
GGGCCTCGTGATACGCCTATTTTTATAGGTTAATGTCATGATAATAATGGTTTCTT
AGACGTCAGGTGGCACTTTTCGGGGAAATGTGCGCGGAACCCCTATTTGTTTATT
TTTCTAAATACATTCAAATATGTATCCGCTCATGAGACAATAACCCTGATAAATG
CTTCAATAATATTGAAAAAGGAAGAGTATGAGTATTCAACATTTCCGTGTCGCCC
TTATTCCCTTTTTTGCGGCATTTTGCCTTCCTGTTTTTGCTCACCCAGAAACGCTGG
TGAAAGTAAAAGATGCTGAAGATCAGTTGGGTGCACGAGTGGGTTACATCGAAC
TGGATCTCAACAGCGGTAAGATCCTTGAGAGTTTTCGCCCCGAAGAACGTTTTCC
AATGATGAGCACTTTTAAAGTTCTGCTATGTGGCGCGGTATTATCCCGTATTGAC
GCCGGGCAAGAGCAACTCGGTCGCCGCATACACTATTCTCAGAATGACTTGGTTG
AGTACTCACCAGTCACAGAAAAGCATCTTACGGATGGCATGACAGTAAGAGAAT
TATGCAGTGCTGCCATAACCATGAGTGATAACACTGCGGCCAACTTACTTCTGAC
AACGATCGGAGGACCGAAGGAGCTAACCGCTTTTTTGCACAACATGGGGGATCA
TGTAACTCGCCTTGATCGTTGGGAACCGGAGCTGAATGAAGCCATACCAAACGA
CGAGCGTGACACCACGATGCCTGTAGCAATGGCAACAACGTTGCGCAAACTATT
AACTGGCGAACTACTTACTCTAGCTTCCCGGCAACAATTAATAGACTGGATGGAG
GCGGATAAAGTTGCAGGACCACTTCTGCGCTCGGCCCTTCCGGCTGGCTGGTTTA
TTGCTGATAAATCTGGAGCCGGTGAGCGTGGGTCTCGCGGTATCATTGCAGCACT
GGGGCCAGATGGTAAGCCCTCCCGTATCGTAGTTATCTACACGACGGGGAGTCA
GGCAACTATGGATGAACGAAATAGACAGATCGCTGAGATAGGTGCCTCACTGAT
TAAGCATTGGTAACTGTCAGACCAAGTTTACTCATATATACTTTAGATTGATTTA
AAACTTCATTTTTAATTTAAAAGGATCTAGGTGAAGATCCTTTTTGATAATCTCAT
GACCAAAATCCCTTAACGTGAGTTTTCGTTCCACTGAGCGTCAGACCCCGTAGAA
AAGATCAAAGGATCTTCTTGAGATCCTTTTTTTCTGCGCGTAATCTGCTGCTTGCA
AACAAAAAAACCACCGCTACCAGCGGTGGTTTGTTTGCCGGATCAAGAGCTACC
AACTCTTTTTCCGAAGGTAACTGGCTTCAGCAGAGCGCAGATACCAAATACTGTT
CTTCTAGTGTAGCCGTAGTTAGGCCACCACTTCAAGAACTCTGTAGCACCGCCTA
CATACCTCGCTCTGCTAATCCTGTTACCAGTGGCTGCTGCCAGTGGCGATAAGTC
GTGTCTTACCGGGTTGGACTCAAGACGATAGTTACCGGATAAGGCGCAGCGGTC
GGGCTGAACGGGGGGTTCGTGCACACAGCCCAGCTTGGAGCGAACGACCTACAC
CGAACTGAGATACCTACAGCGTGAGCTATGAGAAAGCGCCACGCTTCCCGAAGG
GAGAAAGGCGGACAGGTATCCGGTAAGCGGCAGGGTCGGAACAGGAGAGCGCA
CGAGGGAGCTTCCAGGGGGAAACGCCTGGTATCTTTATAGTCCTGTCGGGTTTCG
CCACCTCTGACTTGAGCGTCGATTTTTGTGATGCTCGTCAGGGGGGCGGAGCCTA
TGGAAAAACGCCAGCAACGCGGCCTTTTTACGGTTCCTGGCCTTTTGCTGGCCTT
TTGCTCACATGTTCTTTCCTGCGTTATCCCCTGATTCTGTGGATAACCGTATTACC
GCCTTTGAGTGAGCTGATACCGCTCGCCGCAGCCGAACGACCGAGCGCAGCGAG
TCAGTGAGCGAGGAAGCGGAAGAGCGCCCAATACGCAAACCGCCTCTCCCCGCG
CGTTGGCCGATTCATTAATGCAGCTGGCACGACAGGTTTCCCGACTGGAAAGCGG
GCAGTGAGCGCAACGCAATTAATGTGAGTTAGCTCACTCATTAGGCACCCCAGG
CTTTACACTTTATGCTTCCGGCTCGTATGTTGTGTGGAATTGTGAGCGGATAACA
ATTTCACACAGGAAACAGCTATGACCATGATTACGCCAAGCTCTCGAGATCTAGA
AAGCTTCCCGGGGGGATCTG
SEQ ID NO: 12
pscAAV EF1s MMAB WT RBG 5051
GGCCACTCCCTCTCTGCGCGCTCGCTCGCTCACTGAGGCCGGGCGACCAAAGGTC
GCCCGGCCTCAGTGAGCGAGCGAGCGCGCAGAGAGGGAGTGGCCAACTCCATCA
CTAGGGGTTCCTGGAGGGGTGGAGTCGTGACCTAGGGGCTCCGGTGCCCGTCAG
TGGGCAGAGCGCACATCGCCCACAGTCCCCGAGAAGTTGGGGGGAGGGGTCGGC
AATTGAACCGGTGCCTAGAGAAGGTGGCGCGGGGTAAACTGGGAAAGTGATGTC
GTGTACTGGCTCCGCCTTTTTCCCGAGGGTGGGGGAGAACCGTATATAAGTGCAG
TAGTCGCCGTGAACGTTCTTTTTCGCAACGGGTTTGCCGCCAGAACACAGGTCAG
ATCAGATCTTTGTCGATCCTACCATCCACTCGACACACCCGCCAGCGGCCGCGTT
GGTATCAAGGTTACAAGACAGGTTTAAGGAGACCAATAGAAACTGGGCATGTGG
AGACAGAGAAGACTCTTGGGTTTCTGATAGGCACTGACTCTCTTCCTTTGTCCTGT
TCCCATTTCAGAAGCTTCCGAGCTCTCGAATTAAATAAGAGAGAAAAGAAGAGT
AAGAAGAAATATAAGAGCCACCATGGCTGTGTGCGGCCTGGGGAGCCGTCTTGG
CCTGGGGAGCCGTCTTGGCCTGCGCGGGTGCTTCGGCGCCGCCAGGCTCCTGTAT
CCCCGTTTCCAGAGCCGCGGCCCTCAGGGCGTGGAAGACGGGGACAGGCCACAG
CCTTCCTCGAAGACACCCAGGATCCCCAAGATTTACACCAAAACGGGAGACAAA
GGGTTTTCTAGTACCTTCACAGGAGAAAGGAGACCCAAAGATGACCAAGTGTTT
GAAGCCGTGGGAACTACAGATGAATTAAGTTCAGCTATTGGGTTTGCTCTGGAAT
TAGTCACAGAAAAGGGCCATACATTTGCCGAAGAGCTTCAGAAAATCCAGTGCA
CATTGCAGGACGTCGGCTCGGCCCTGGCGACACCATGCTCCTCGGCCCGGGAGG
CTCACTTAAAGTATACCACGTTCAAGGCGGGGCCCATCCTGGAGCTGGAGCAGT
GGATCGACAAGTACACCAGCCAGCTCCCACCACTCACGGCCTTCATCCTGCCTTC
GGGAGGCAAGATCAGCTCGGCGCTGCATTTCTGCCGGGCCGTGTGCCGCCGGGC
CGAGAGACGTGTGGTGCCTCTTGTCCAGATGGGAGAGACCGATGCGAACGTGGC
CAAGTTCTTAAACAGACTCAGTGACTATCTCTTCACGCTAGCCAGATATGCAGCC
ATGAAGGAGGGGAATCAAGAGAAAATATACATGAAAAATGACCCATCGGCCGA
GTCTGAGGGACTCTGAACTCGAGATAACAGGCCTATTGATTGGAAAGTTTGTCAA
CGAATTGTGGGTCTTTTGGGGTTTGCTGCCCCTTTTACGCAATGTGGATATCCTGC
TTTAATGCCTTTATATGCATGTATACAAGCAAAACAGGCTTTTACTTTCTCGCCAA
CTTACAAGGCCTTTCTCAGTAAACAGTATATGACCCTTTACCCCGTTGCTCGGCA
ACGGCCTGGTCTGTGCCAAGTGTTTGCTGACGCAACCCCCACTGGTTGGGGCTTG
GCCATAGGCCATCAGCGCATGCGTGGAACCTTTGTGTCTCCTCTGCCGATCCATA
CTGCGGAACTCCTAGCCGCTTGTTTTGCTCGCAGCAGGTCTGGAGCAAACCTCAT
CGGGACCGACAATTCTGTCGTACTCTCCCGCAAGTATACATCGTTTCCATGGCTG
CTAGGCTGTGCTGCCAACTGGATCCTGCGCGGGACGTCCTTTGTTTACGTCCCGT
CGGCGCTGAATCCCGCGGACGACCCCTCCCGGGGCCGCTTGGGGCTCTACCGCCC
GCTTCTCCGTCTGCCGTACCGTCCGACCACGGGGCGCACCTCTCTTTACGCGGAC
TCCCCGTCTGTGCCTTCTCATCTGCCGGACCGTGTGCACTTCGCTTCACCTCTGCA
CGTCGCATGGAGGCCACCGTGAACGCCCACCGGAACCTGCCCAAGGTCTTGCAT
AAGAGGACTCTTGGACTTTCAGCAATGTCATCTCCGATCTTTTTCCCTCTGCCAAA
AATTATGGGGACATCATGAAGCCCCTTGAGCATCTGACTTCTGGCTAATAAAGGA
AATTTATTTTCATTGCAATAGTGTGTTGGAATTTTTTGTGTCTCTCACACTAGTCC
ACTCCCTCTCTGCGCGCTCGCTCGCTCACTGAGGCCGGGCGACCAAAGGTCGCCC
GACGCCCGGGCTTTGCCCGGGCGGCCTCAGTGAGCGAGCGAGCGCGCAGAGAGG
GACAGATCCGGGCCCGCATGCGTCGACAATTCACTGGCCGTCGTTTTACAACGTC
GTGACTGGGAAAACCCTGGCGTTACCCAACTTAATCGCCTTGCAGCACATCCCCC
TTTCGCCAGCTGGCGTAATAGCGAAGAGGCCCGCACCGATCGCCCTTCCCAACA
GTTGCGCAGCCTGAATGGCGAATGGCGCCTGATGCGGTATTTTCTCCTTACGCAT
CTGTGCGGTATTTCACACCGCATATGGTGCACTCTCAGTACAATCTGCTCTGATG
CCGCATAGTTAAGCCAGCCCCGACACCCGCCAACACCCGCTGACGCGCCCTGAC
GGGCTTGTCTGCTCCCGGCATCCGCTTACAGACAAGCTGTGACCGTCTCCGGGAG
CTGCATGTGTCAGAGGTTTTCACCGTCATCACCGAAACGCGCGAGACGAAAGGG
CCTCGTGATACGCCTATTTTTATAGGTTAATGTCATGATAATAATGGTTTCTTAGA
CGTCAGGTGGCACTTTTCGGGGAAATGTGCGCGGAACCCCTATTTGTTTATTTTTC
TAAATACATTCAAATATGTATCCGCTCATGAGACAATAACCCTGATAAATGCTTC
AATAATATTGAAAAAGGAAGAGTATGAGTATTCAACATTTCCGTGTCGCCCTTAT
TCCCTTTTTTGCGGCATTTTGCCTTCCTGTTTTTGCTCACCCAGAAACGCTGGTGA
AAGTAAAAGATGCTGAAGATCAGTTGGGTGCACGAGTGGGTTACATCGAACTGG
ATCTCAACAGCGGTAAGATCCTTGAGAGTTTTCGCCCCGAAGAACGTTTTCCAAT
GATGAGCACTTTTAAAGTTCTGCTATGTGGCGCGGTATTATCCCGTATTGACGCC
GGGCAAGAGCAACTCGGTCGCCGCATACACTATTCTCAGAATGACTTGGTTGAGT
ACTCACCAGTCACAGAAAAGCATCTTACGGATGGCATGACAGTAAGAGAATTAT
GCAGTGCTGCCATAACCATGAGTGATAACACTGCGGCCAACTTACTTCTGACAAC
GATCGGAGGACCGAAGGAGCTAACCGCTTTTTTGCACAACATGGGGGATCATGT
AACTCGCCTTGATCGTTGGGAACCGGAGCTGAATGAAGCCATACCAAACGACGA
GCGTGACACCACGATGCCTGTAGCAATGGCAACAACGTTGCGCAAACTATTAAC
TGGCGAACTACTTACTCTAGCTTCCCGGCAACAATTAATAGACTGGATGGAGGCG
GATAAAGTTGCAGGACCACTTCTGCGCTCGGCCCTTCCGGCTGGCTGGTTTATTG
CTGATAAATCTGGAGCCGGTGAGCGTGGGTCTCGCGGTATCATTGCAGCACTGGG
GCCAGATGGTAAGCCCTCCCGTATCGTAGTTATCTACACGACGGGGAGTCAGGC
AACTATGGATGAACGAAATAGACAGATCGCTGAGATAGGTGCCTCACTGATTAA
GCATTGGTAACTGTCAGACCAAGTTTACTCATATATACTTTAGATTGATTTAAAA
CTTCATTTTTAATTTAAAAGGATCTAGGTGAAGATCCTTTTTGATAATCTCATGAC
CAAAATCCCTTAACGTGAGTTTTCGTTCCACTGAGCGTCAGACCCCGTAGAAAAG
ATCAAAGGATCTTCTTGAGATCCTTTTTTTCTGCGCGTAATCTGCTGCTTGCAAAC
AAAAAAACCACCGCTACCAGCGGTGGTTTGTTTGCCGGATCAAGAGCTACCAAC
TCTTTTTCCGAAGGTAACTGGCTTCAGCAGAGCGCAGATACCAAATACTGTTCTT
CTAGTGTAGCCGTAGTTAGGCCACCACTTCAAGAACTCTGTAGCACCGCCTACAT
ACCTCGCTCTGCTAATCCTGTTACCAGTGGCTGCTGCCAGTGGCGATAAGTCGTG
TCTTACCGGGTTGGACTCAAGACGATAGTTACCGGATAAGGCGCAGCGGTCGGG
CTGAACGGGGGGTTCGTGCACACAGCCCAGCTTGGAGCGAACGACCTACACCGA
ACTGAGATACCTACAGCGTGAGCTATGAGAAAGCGCCACGCTTCCCGAAGGGAG
AAAGGCGGACAGGTATCCGGTAAGCGGCAGGGTCGGAACAGGAGAGCGCACGA
GGGAGCTTCCAGGGGGAAACGCCTGGTATCTTTATAGTCCTGTCGGGTTTCGCCA
CCTCTGACTTGAGCGTCGATTTTTGTGATGCTCGTCAGGGGGGCGGAGCCTATGG
AAAAACGCCAGCAACGCGGCCTTTTTACGGTTCCTGGCCTTTTGCTGGCCTTTTGC
TCACATGTTCTTTCCTGCGTTATCCCCTGATTCTGTGGATAACCGTATTACCGCCT
TTGAGTGAGCTGATACCGCTCGCCGCAGCCGAACGACCGAGCGCAGCGAGTCAG
TGAGCGAGGAAGCGGAAGAGCGCCCAATACGCAAACCGCCTCTCCCCGCGCGTT
GGCCGATTCATTAATGCAGCTGGCACGACAGGTTTCCCGACTGGAAAGCGGGCA
GTGAGCGCAACGCAATTAATGTGAGTTAGCTCACTCATTAGGCACCCCAGGCTTT
ACACTTTATGCTTCCGGCTCGTATGTTGTGTGGAATTGTGAGCGGATAACAATTT
CACACAGGAAACAGCTATGACCATGATTACGCCAAGCTCTCGAGATCTAGAAAG
CTTCCCGGGGGGATCTG
SEQ ID NO: 13
pscAAV EF1s synMMAB1 5051
GGCCACTCCCTCTCTGCGCGCTCGCTCGCTCACTGAGGCCGGGCGACCAAAGGTC
GCCCGGCCTCAGTGAGCGAGCGAGCGCGCAGAGAGGGAGTGGCCAACTCCATCA
CTAGGGGTTCCTGGAGGGGTGGAGTCGTGACCTAGGGGCTCCGGTGCCCGTCAG
TGGGCAGAGCGCACATCGCCCACAGTCCCCGAGAAGTTGGGGGGAGGGGTCGGC
AATTGAACCGGTGCCTAGAGAAGGTGGCGCGGGGTAAACTGGGAAAGTGATGTC
GTGTACTGGCTCCGCCTTTTTCCCGAGGGTGGGGGAGAACCGTATATAAGTGCAG
TAGTCGCCGTGAACGTTCTTTTTCGCAACGGGTTTGCCGCCAGAACACAGGTCAG
ATCAGATCTTTGTCGATCCTACCATCCACTCGACACACCCGCCAGCGGCCGCGTT
GGTATCAAGGTTACAAGACAGGTTTAAGGAGACCAATAGAAACTGGGCATGTGG
AGACAGAGAAGACTCTTGGGTTTCTGATAGGCACTGACTCTCTTCCTTTGTCCTGT
TCCCATTTCAGAAGCTTCCGAGCTCTCGAATTAAATAAGAGAGAAAAGAAGAGT
AAGAAGAAATATAAGAGCCACCATGGCTGTCTGCGGTCTGGGCAGCAGACTGGG
TCTTGGGTCCCGCTTGGGACTCCGAGGCTGCTTTGGTGCAGCACGCTTGCTGTAT
CCTCGCTTCCAGTCTAGAGGGCCACAAGGAGTAGAGGACGGTGATCGGCCCCAG
CCAAGCAGCAAAACACCGAGGATACCGAAAATTTATACAAAGACGGGAGATAA
GGGGTTTAGCAGCACCTTCACGGGCGAGCGACGGCCCAAGGACGACCAAGTATT
CGAGGCAGTCGGGACGACGGACGAACTGTCAAGTGCTATTGGGTTCGCTCTGGA
GCTGGTTACGGAGAAGGGACATACTTTCGCGGAGGAGCTCCAAAAAATACAATG
CACCCTTCAAGACGTGGGCAGTGCCCTGGCGACACCGTGTAGCAGCGCGCGCGA
GGCCCACTTGAAATATACAACTTTCAAAGCAGGACCTATATTGGAACTCGAGCA
GTGGATAGACAAATACACGTCCCAACTGCCTCCCCTGACAGCTTTTATCTTGCCA
TCTGGCGGTAAGATAAGTTCTGCTCTTCATTTCTGTAGGGCAGTATGTAGGCGAG
CGGAAAGGCGGGTTGTACCCCTGGTGCAGATGGGTGAGACTGACGCCAATGTGG
CCAAGTTCTTGAACCGCCTGTCTGATTATCTGTTTACACTCGCTAGATACGCCGCT
ATGAAAGAAGGAAATCAAGAGAAGATATACATGAAGAACGATCCGTCAGCGGA
ATCCGAGGGGCTCTGAACTCGAGATAACAGGCCTATTGATTGGAAAGTTTGTCAA
CGAATTGTGGGTCTTTTGGGGTTTGCTGCCCCTTTTACGCAATGTGGATATCCTGC
TTTAATGCCTTTATATGCATGTATACAAGCAAAACAGGCTTTTACTTTCTCGCCAA
CTTACAAGGCCTTTCTCAGTAAACAGTATATGACCCTTTACCCCGTTGCTCGGCA
ACGGCCTGGTCTGTGCCAAGTGTTTGCTGACGCAACCCCCACTGGTTGGGGCTTG
GCCATAGGCCATCAGCGCATGCGTGGAACCTTTGTGTCTCCTCTGCCGATCCATA
CTGCGGAACTCCTAGCCGCTTGTTTTGCTCGCAGCAGGTCTGGAGCAAACCTCAT
CGGGACCGACAATTCTGTCGTACTCTCCCGCAAGTATACATCGTTTCCATGGCTG
CTAGGCTGTGCTGCCAACTGGATCCTGCGCGGGACGTCCTTTGTTTACGTCCCGT
CGGCGCTGAATCCCGCGGACGACCCCTCCCGGGGCCGCTTGGGGCTCTACCGCCC
GCTTCTCCGTCTGCCGTACCGTCCGACCACGGGGCGCACCTCTCTTTACGCGGAC
TCCCCGTCTGTGCCTTCTCATCTGCCGGACCGTGTGCACTTCGCTTCACCTCTGCA
CGTCGCATGGAGGCCACCGTGAACGCCCACCGGAACCTGCCCAAGGTCTTGCAT
AAGAGGACTCTTGGACTTTCAGCAATGTCATCTCCGATCTTTTTCCCTCTGCCAAA
AATTATGGGGACATCATGAAGCCCCTTGAGCATCTGACTTCTGGCTAATAAAGGA
AATTTATTTTCATTGCAATAGTGTGTTGGAATTTTTTGTGTCTCTCACACTAGTCC
ACTCCCTCTCTGCGCGCTCGCTCGCTCACTGAGGCCGGGCGACCAAAGGTCGCCC
GACGCCCGGGCTTTGCCCGGGCGGCCTCAGTGAGCGAGCGAGCGCGCAGAGAGG
GACAGATCCGGGCCCGCATGCGTCGACAATTCACTGGCCGTCGTTTTACAACGTC
GTGACTGGGAAAACCCTGGCGTTACCCAACTTAATCGCCTTGCAGCACATCCCCC
TTTCGCCAGCTGGCGTAATAGCGAAGAGGCCCGCACCGATCGCCCTTCCCAACA
GTTGCGCAGCCTGAATGGCGAATGGCGCCTGATGCGGTATTTTCTCCTTACGCAT
CTGTGCGGTATTTCACACCGCATATGGTGCACTCTCAGTACAATCTGCTCTGATG
CCGCATAGTTAAGCCAGCCCCGACACCCGCCAACACCCGCTGACGCGCCCTGAC
GGGCTTGTCTGCTCCCGGCATCCGCTTACAGACAAGCTGTGACCGTCTCCGGGAG
CTGCATGTGTCAGAGGTTTTCACCGTCATCACCGAAACGCGCGAGACGAAAGGG
CCTCGTGATACGCCTATTTTTATAGGTTAATGTCATGATAATAATGGTTTCTTAGA
CGTCAGGTGGCACTTTTCGGGGAAATGTGCGCGGAACCCCTATTTGTTTATTTTTC
TAAATACATTCAAATATGTATCCGCTCATGAGACAATAACCCTGATAAATGCTTC
AATAATATTGAAAAAGGAAGAGTATGAGTATTCAACATTTCCGTGTCGCCCTTAT
TCCCTTTTTTGCGGCATTTTGCCTTCCTGTTTTTGCTCACCCAGAAACGCTGGTGA
AAGTAAAAGATGCTGAAGATCAGTTGGGTGCACGAGTGGGTTACATCGAACTGG
ATCTCAACAGCGGTAAGATCCTTGAGAGTTTTCGCCCCGAAGAACGTTTTCCAAT
GATGAGCACTTTTAAAGTTCTGCTATGTGGCGCGGTATTATCCCGTATTGACGCC
GGGCAAGAGCAACTCGGTCGCCGCATACACTATTCTCAGAATGACTTGGTTGAGT
ACTCACCAGTCACAGAAAAGCATCTTACGGATGGCATGACAGTAAGAGAATTAT
GCAGTGCTGCCATAACCATGAGTGATAACACTGCGGCCAACTTACTTCTGACAAC
GATCGGAGGACCGAAGGAGCTAACCGCTTTTTTGCACAACATGGGGGATCATGT
AACTCGCCTTGATCGTTGGGAACCGGAGCTGAATGAAGCCATACCAAACGACGA
GCGTGACACCACGATGCCTGTAGCAATGGCAACAACGTTGCGCAAACTATTAAC
TGGCGAACTACTTACTCTAGCTTCCCGGCAACAATTAATAGACTGGATGGAGGCG
GATAAAGTTGCAGGACCACTTCTGCGCTCGGCCCTTCCGGCTGGCTGGTTTATTG
CTGATAAATCTGGAGCCGGTGAGCGTGGGTCTCGCGGTATCATTGCAGCACTGGG
GCCAGATGGTAAGCCCTCCCGTATCGTAGTTATCTACACGACGGGGAGTCAGGC
AACTATGGATGAACGAAATAGACAGATCGCTGAGATAGGTGCCTCACTGATTAA
GCATTGGTAACTGTCAGACCAAGTTTACTCATATATACTTTAGATTGATTTAAAA
CTTCATTTTTAATTTAAAAGGATCTAGGTGAAGATCCTTTTTGATAATCTCATGAC
CAAAATCCCTTAACGTGAGTTTTCGTTCCACTGAGCGTCAGACCCCGTAGAAAAG
ATCAAAGGATCTTCTTGAGATCCTTTTTTTCTGCGCGTAATCTGCTGCTTGCAAAC
AAAAAAACCACCGCTACCAGCGGTGGTTTGTTTGCCGGATCAAGAGCTACCAAC
TCTTTTTCCGAAGGTAACTGGCTTCAGCAGAGCGCAGATACCAAATACTGTTCTT
CTAGTGTAGCCGTAGTTAGGCCACCACTTCAAGAACTCTGTAGCACCGCCTACAT
ACCTCGCTCTGCTAATCCTGTTACCAGTGGCTGCTGCCAGTGGCGATAAGTCGTG
TCTTACCGGGTTGGACTCAAGACGATAGTTACCGGATAAGGCGCAGCGGTCGGG
CTGAACGGGGGGTTCGTGCACACAGCCCAGCTTGGAGCGAACGACCTACACCGA
ACTGAGATACCTACAGCGTGAGCTATGAGAAAGCGCCACGCTTCCCGAAGGGAG
AAAGGCGGACAGGTATCCGGTAAGCGGCAGGGTCGGAACAGGAGAGCGCACGA
GGGAGCTTCCAGGGGGAAACGCCTGGTATCTTTATAGTCCTGTCGGGTTTCGCCA
CCTCTGACTTGAGCGTCGATTTTTGTGATGCTCGTCAGGGGGGCGGAGCCTATGG
AAAAACGCCAGCAACGCGGCCTTTTTACGGTTCCTGGCCTTTTGCTGGCCTTTTGC
TCACATGTTCTTTCCTGCGTTATCCCCTGATTCTGTGGATAACCGTATTACCGCCT
TTGAGTGAGCTGATACCGCTCGCCGCAGCCGAACGACCGAGCGCAGCGAGTCAG
TGAGCGAGGAAGCGGAAGAGCGCCCAATACGCAAACCGCCTCTCCCCGCGCGTT
GGCCGATTCATTAATGCAGCTGGCACGACAGGTTTCCCGACTGGAAAGCGGGCA
GTGAGCGCAACGCAATTAATGTGAGTTAGCTCACTCATTAGGCACCCCAGGCTTT
ACACTTTATGCTTCCGGCTCGTATGTTGTGTGGAATTGTGAGCGGATAACAATTT
CACACAGGAAACAGCTATGACCATGATTACGCCAAGCTCTCGAGATCTAGAAAG
CTTCCCGGGGGGATCTG
SEQ ID NO: 14
pscAAV EF1s synMMAB2 5019
GGCCACTCCCTCTCTGCGCGCTCGCCCGGCCTCAGTGAGCGAGCGAGCGCGCAG
AGAGGGAGTGGCCAACTCCATCACTAGGGGTTCCTGGAGGGGTGGAGTCGTGAC
CTAGGGGCTCCGGTGCCCGTCAGTGGGCAGAGCGCACATCGCCCACAGTCCCCG
AGAAGTTGGGGGGAGGGGTCGGCAATTGAACCGGTGCCTAGAGAAGGTGGCGC
GGGGTAAACTGGGAAAGTGATGTCGTGTACTGGCTCCGCCTTTTTCCCGAGGGTG
GGGGAGAACCGTATATAAGTGCAGTAGTCGCCGTGAACGTTCTTTTTCGCAACGG
GTTTGCCGCCAGAACACAGGTCAGATCAGATCTTTGTCGATCCTACCATCCACTC
GACACACCCGCCAGCGGCCGCGTTGGTATCAAGGTTACAAGACAGGTTTAAGGA
GACCAATAGAAACTGGGCATGTGGAGACAGAGAAGACTCTTGGGTTTCTGATAG
GCACTGACTCTCTTCCTTTGTCCTGTTCCCATTTCAGAAGCTTCCGAGCTCTCGAA
TTAAATAAGAGAGAAAAGAAGAGTAAGAAGAAATATAAGAGCCACCATGGCGG
TCTGCGGATTGGGTTCACGATTGGGCCTTGGGTCTAGGCTCGGCCTGCGAGGTTG
TTTCGGAGCTGCTAGATTGTTGTACCCACGATTCCAAAGCAGGGGACCACAAGGT
GTGGAAGACGGTGACAGGCCTCAGCCCTCAAGCAAAACCCCTCGGATTCCGAAA
ATATATACAAAGACAGGAGACAAAGGCTTTTCAAGCACGTTTACTGGCGAACGA
AGGCCTAAAGACGATCAAGTTTTTGAGGCGGTAGGGACAACTGACGAACTTTCA
TCTGCGATAGGCTTCGCGCTCGAGTTGGTGACGGAGAAGGGTCATACATTCGCTG
AAGAACTTCAGAAAATTCAGTGTACGCTCCAAGATGTTGGCAGTGCGCTTGCGAC
ACCTTGCAGCTCAGCAAGGGAAGCGCATTTGAAGTACACCACTTTCAAAGCGGG
CCCGATTCTCGAACTTGAACAATGGATTGACAAATATACAAGCCAACTGCCGCCT
CTTACCGCCTTCATCCTCCCTAGTGGTGGCAAAATTTCTTCCGCGCTTCATTTTTG
TAGGGCCGTGTGTCGCCGCGCCGAGAGACGGGTGGTTCCGTTGGTACAGATGGG
TGAAACAGATGCAAACGTGGCAAAGTTCCTTAACCGCCTCAGCGACTACTTGTTC
ACTCTCGCTAGGTATGCCGCCATGAAAGAAGGGAATCAAGAGAAGATCTATATG
AAGAATGACCCGAGCGCTGAAAGCGAGGGACTTTGAACTCGAGATAACAGGCCT
ATTGATTGGAAAGTTTGTCAACGAATTGTGGGTCTTTTGGGGTTTGCTGCCCCTTT
TACGCAATGTGGATATCCTGCTTTAATGCCTTTATATGCATGTATACAAGCAAAA
CAGGCTTTTACTTTCTCGCCAACTTACAAGGCCTTTCTCAGTAAACAGTATATGAC
CCTTTACCCCGTTGCTCGGCAACGGCCTGGTCTGTGCCAAGTGTTTGCTGACGCA
ACCCCCACTGGTTGGGGCTTGGCCATAGGCCATCAGCGCATGCGTGGAACCTTTG
TGTCTCCTCTGCCGATCCATACTGCGGAACTCCTAGCCGCTTGTTTTGCTCGCAGC
AGGTCTGGAGCAAACCTCATCGGGACCGACAATTCTGTCGTACTCTCCCGCAAGT
ATACATCGTTTCCATGGCTGCTAGGCTGTGCTGCCAACTGGATCCTGCGCGGGAC
GTCCTTTGTTTACGTCCCGTCGGCGCTGAATCCCGCGGACGACCCCTCCCGGGGC
CGCTTGGGGCTCTACCGCCCGCTTCTCCGTCTGCCGTACCGTCCGACCACGGGGC
GCACCTCTCTTTACGCGGACTCCCCGTCTGTGCCTTCTCATCTGCCGGACCGTGTG
CACTTCGCTTCACCTCTGCACGTCGCATGGAGGCCACCGTGAACGCCCACCGGAA
CCTGCCCAAGGTCTTGCATAAGAGGACTCTTGGACTTTCAGCAATGTCATCTCCG
ATCTTTTTCCCTCTGCCAAAAATTATGGGGACATCATGAAGCCCCTTGAGCATCT
GACTTCTGGCTAATAAAGGAAATTTATTTTCATTGCAATAGTGTGTTGGAATTTTT
TGTGTCTCTCACACTAGTCCACTCCCTCTCTGCGCGCTCGCTCGCTCACTGAGGCC
GGGCGACCAAAGGTCGCCCGACGCCCGGGCTTTGCCCGGGCGGCCTCAGTGAGC
GAGCGAGCGCGCAGAGAGGGACAGATCCGGGCCCGCATGCGTCGACAATTCACT
GGCCGTCGTTTTACAACGTCGTGACTGGGAAAACCCTGGCGTTACCCAACTTAAT
CGCCTTGCAGCACATCCCCCTTTCGCCAGCTGGCGTAATAGCGAAGAGGCCCGCA
CCGATCGCCCTTCCCAACAGTTGCGCAGCCTGAATGGCGAATGGCGCCTGATGCG
GTATTTTCTCCTTACGCATCTGTGCGGTATTTCACACCGCATATGGTGCACTCTCA
GTACAATCTGCTCTGATGCCGCATAGTTAAGCCAGCCCCGACACCCGCCAACACC
CGCTGACGCGCCCTGACGGGCTTGTCTGCTCCCGGCATCCGCTTACAGACAAGCT
GTGACCGTCTCCGGGAGCTGCATGTGTCAGAGGTTTTCACCGTCATCACCGAAAC
GCGCGAGACGAAAGGGCCTCGTGATACGCCTATTTTTATAGGTTAATGTCATGAT
AATAATGGTTTCTTAGACGTCAGGTGGCACTTTTCGGGGAAATGTGCGCGGAACC
CCTATTTGTTTATTTTTCTAAATACATTCAAATATGTATCCGCTCATGAGACAATA
ACCCTGATAAATGCTTCAATAATATTGAAAAAGGAAGAGTATGAGTATTCAACA
TTTCCGTGTCGCCCTTATTCCCTTTTTTGCGGCATTTTGCCTTCCTGTTTTTGCTCA
CCCAGAAACGCTGGTGAAAGTAAAAGATGCTGAAGATCAGTTGGGTGCACGAGT
GGGTTACATCGAACTGGATCTCAACAGCGGTAAGATCCTTGAGAGTTTTCGCCCC
GAAGAACGTTTTCCAATGATGAGCACTTTTAAAGTTCTGCTATGTGGCGCGGTAT
TATCCCGTATTGACGCCGGGCAAGAGCAACTCGGTCGCCGCATACACTATTCTCA
GAATGACTTGGTTGAGTACTCACCAGTCACAGAAAAGCATCTTACGGATGGCAT
GACAGTAAGAGAATTATGCAGTGCTGCCATAACCATGAGTGATAACACTGCGGC
CAACTTACTTCTGACAACGATCGGAGGACCGAAGGAGCTAACCGCTTTTTTGCAC
AACATGGGGGATCATGTAACTCGCCTTGATCGTTGGGAACCGGAGCTGAATGAA
GCCATACCAAACGACGAGCGTGACACCACGATGCCTGTAGCAATGGCAACAACG
TTGCGCAAACTATTAACTGGCGAACTACTTACTCTAGCTTCCCGGCAACAATTAA
TAGACTGGATGGAGGCGGATAAAGTTGCAGGACCACTTCTGCGCTCGGCCCTTCC
GGCTGGCTGGTTTATTGCTGATAAATCTGGAGCCGGTGAGCGTGGGTCTCGCGGT
ATCATTGCAGCACTGGGGCCAGATGGTAAGCCCTCCCGTATCGTAGTTATCTACA
CGACGGGGAGTCAGGCAACTATGGATGAACGAAATAGACAGATCGCTGAGATAG
GTGCCTCACTGATTAAGCATTGGTAACTGTCAGACCAAGTTTACTCATATATACT
TTAGATTGATTTAAAACTTCATTTTTAATTTAAAAGGATCTAGGTGAAGATCCTTT
TTGATAATCTCATGACCAAAATCCCTTAACGTGAGTTTTCGTTCCACTGAGCGTC
AGACCCCGTAGAAAAGATCAAAGGATCTTCTTGAGATCCTTTTTTTCTGCGCGTA
ATCTGCTGCTTGCAAACAAAAAAACCACCGCTACCAGCGGTGGTTTGTTTGCCGG
ATCAAGAGCTACCAACTCTTTTTCCGAAGGTAACTGGCTTCAGCAGAGCGCAGAT
ACCAAATACTGTTCTTCTAGTGTAGCCGTAGTTAGGCCACCACTTCAAGAACTCT
GTAGCACCGCCTACATACCTCGCTCTGCTAATCCTGTTACCAGTGGCTGCTGCCA
GTGGCGATAAGTCGTGTCTTACCGGGTTGGACTCAAGACGATAGTTACCGGATAA
GGCGCAGCGGTCGGGCTGAACGGGGGGTTCGTGCACACAGCCCAGCTTGGAGCG
AACGACCTACACCGAACTGAGATACCTACAGCGTGAGCTATGAGAAAGCGCCAC
GCTTCCCGAAGGGAGAAAGGCGGACAGGTATCCGGTAAGCGGCAGGGTCGGAA
CAGGAGAGCGCACGAGGGAGCTTCCAGGGGGAAACGCCTGGTATCTTTATAGTC
CTGTCGGGTTTCGCCACCTCTGACTTGAGCGTCGATTTTTGTGATGCTCGTCAGGG
GGGCGGAGCCTATGGAAAAACGCCAGCAACGCGGCCTTTTTACGGTTCCTGGCCT
TTTGCTGGCCTTTTGCTCACATGTTCTTTCCTGCGTTATCCCCTGATTCTGTGGATA
ACCGTATTACCGCCTTTGAGTGAGCTGATACCGCTCGCCGCAGCCGAACGACCGA
GCGCAGCGAGTCAGTGAGCGAGGAAGCGGAAGAGCGCCCAATACGCAAACCGC
CTCTCCCCGCGCGTTGGCCGATTCATTAATGCAGCTGGCACGACAGGTTTCCCGA
CTGGAAAGCGGGCAGTGAGCGCAACGCAATTAATGTGAGTTAGCTCACTCATTA
GGCACCCCAGGCTTTACACTTTATGCTTCCGGCTCGTATGTTGTGTGGAATTGTGA
GCGGATAACAATTTCACACAGGAAACAGCTATGACCATGATTACGCCAAGCTCT
CGAGATCTAGAAAGCTTCCCGGGGGGATCTG
SEQ ID NO: 15
pscAAV EF1s synMMAB3 5051
GGCCACTCCCTCTCTGCGCGCTCGCTCGCTCACTGAGGCCGGGCGACCAAAGGTC
GCCCGGCCTCAGTGAGCGAGCGAGCGCGCAGAGAGGGAGTGGCCAACTCCATCA
CTAGGGGTTCCTGGAGGGGTGGAGTCGTGACCTAGGGGCTCCGGTGCCCGTCAG
TGGGCAGAGCGCACATCGCCCACAGTCCCCGAGAAGTTGGGGGGAGGGGTCGGC
AATTGAACCGGTGCCTAGAGAAGGTGGCGCGGGGTAAACTGGGAAAGTGATGTC
GTGTACTGGCTCCGCCTTTTTCCCGAGGGTGGGGGAGAACCGTATATAAGTGCAG
TAGTCGCCGTGAACGTTCTTTTTCGCAACGGGTTTGCCGCCAGAACACAGGTCAG
ATCAGATCTTTGTCGATCCTACCATCCACTCGACACACCCGCCAGCGGCCGCGTT
GGTATCAAGGTTACAAGACAGGTTTAAGGAGACCAATAGAAACTGGGCATGTGG
AGACAGAGAAGACTCTTGGGTTTCTGATAGGCACTGACTCTCTTCCTTTGTCCTGT
TCCCATTTCAGAAGCTTCCGAGCTCTCGAATTAAATAAGAGAGAAAAGAAGAGT
AAGAAGAAATATAAGAGCCACCATGGCCGTGTGCGGCCTGGGCAGCAGACTGGG
ACTGGGTTCCCGGCTGGGACTGAGAGGCTGCTTCGGCGCCGCAAGACTGCTGTAC
CCCAGATTCCAGAGCAGAGGACCTCAGGGCGTGGAAGATGGCGATAGACCTCAA
CCTAGCTCAAAGACCCCTAGAATCCCCAAGATCTACACAAAGACAGGCGACAAG
GGCTTTAGCAGCACCTTTACCGGCGAAAGACGGCCTAAGGACGACCAGGTTTTC
GAGGCCGTCGGAACAACCGACGAGCTGAGCTCTGCTATCGGCTTCGCCCTCGAG
CTCGTGACCGAGAAGGGCCACACATTTGCCGAGGAACTGCAAAAAATTCAGTGT
ACCCTGCAGGACGTGGGATCTGCTCTGGCCACACCTTGCAGCAGCGCTCGGGAA
GCCCACCTGAAGTACACCACCTTCAAGGCCGGCCCAATCCTGGAACTGGAACAG
TGGATCGACAAGTACACCAGCCAGCTGCCACCTCTGACCGCCTTCATCCTGCCTT
CTGGCGGCAAGATCAGCAGCGCCCTGCACTTCTGCAGAGCCGTGTGTAGAAGGG
CCGAGCGGCGGGTGGTGCCCCTGGTGCAGATGGGCGAGACAGATGCCAACGTGG
CCAAGTTCCTGAATAGACTGTCTGATTACCTGTTCACCCTGGCTCGCTACGCCGC
CATGAAGGAGGGCAACCAGGAGAAAATCTATATGAAAAACGACCCCTCCGCCGA
GAGCGAGGGCCTGTGAACTCGAGATAACAGGCCTATTGATTGGAAAGTTTGTCA
ACGAATTGTGGGTCTTTTGGGGTTTGCTGCCCCTTTTACGCAATGTGGATATCCTG
CTTTAATGCCTTTATATGCATGTATACAAGCAAAACAGGCTTTTACTTTCTCGCCA
ACTTACAAGGCCTTTCTCAGTAAACAGTATATGACCCTTTACCCCGTTGCTCGGC
AACGGCCTGGTCTGTGCCAAGTGTTTGCTGACGCAACCCCCACTGGTTGGGGCTT
GGCCATAGGCCATCAGCGCATGCGTGGAACCTTTGTGTCTCCTCTGCCGATCCAT
ACTGCGGAACTCCTAGCCGCTTGTTTTGCTCGCAGCAGGTCTGGAGCAAACCTCA
TCGGGACCGACAATTCTGTCGTACTCTCCCGCAAGTATACATCGTTTCCATGGCT
GCTAGGCTGTGCTGCCAACTGGATCCTGCGCGGGACGTCCTTTGTTTACGTCCCG
TCGGCGCTGAATCCCGCGGACGACCCCTCCCGGGGCCGCTTGGGGCTCTACCGCC
CGCTTCTCCGTCTGCCGTACCGTCCGACCACGGGGCGCACCTCTCTTTACGCGGA
CTCCCCGTCTGTGCCTTCTCATCTGCCGGACCGTGTGCACTTCGCTTCACCTCTGC
ACGTCGCATGGAGGCCACCGTGAACGCCCACCGGAACCTGCCCAAGGTCTTGCA
TAAGAGGACTCTTGGACTTTCAGCAATGTCATCTCCGATCTTTTTCCCTCTGCCAA
AAATTATGGGGACATCATGAAGCCCCTTGAGCATCTGACTTCTGGCTAATAAAGG
AAATTTATTTTCATTGCAATAGTGTGTTGGAATTTTTTGTGTCTCTCACACTAGTC
CACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAG
GGACAGATCCGGGCCCGCATGCGTCGACAATTCACTGGCCGTCGTTTTACAACGT
CGTGACTGGGAAAACCCTGGCGTTACCCAACTTAATCGCCTTGCAGCACATCCCC
CTTTCGCCAGCTGGCGTAATAGCGAAGAGGCCCGCACCGATCGCCCTTCCCAACA
GTTGCGCAGCCTGAATGGCGAATGGCGCCTGATGCGGTATTTTCTCCTTACGCAT
CTGTGCGGTATTTCACACCGCATATGGTGCACTCTCAGTACAATCTGCTCTGATG
CCGCATAGTTAAGCCAGCCCCGACACCCGCCAACACCCGCTGACGCGCCCTGAC
GGGCTTGTCTGCTCCCGGCATCCGCTTACAGACAAGCTGTGACCGTCTCCGGGAG
CTGCATGTGTCAGAGGTTTTCACCGTCATCACCGAAACGCGCGAGACGAAAGGG
CCTCGTGATACGCCTATTTTTATAGGTTAATGTCATGATAATAATGGTTTCTTAGA
CGTCAGGTGGCACTTTTCGGGGAAATGTGCGCGGAACCCCTATTTGTTTATTTTTC
TAAATACATTCAAATATGTATCCGCTCATGAGACAATAACCCTGATAAATGCTTC
AATAATATTGAAAAAGGAAGAGTATGAGTATTCAACATTTCCGTGTCGCCCTTAT
TCCCTTTTTTGCGGCATTTTGCCTTCCTGTTTTTGCTCACCCAGAAACGCTGGTGA
AAGTAAAAGATGCTGAAGATCAGTTGGGTGCACGAGTGGGTTACATCGAACTGG
ATCTCAACAGCGGTAAGATCCTTGAGAGTTTTCGCCCCGAAGAACGTTTTCCAAT
GATGAGCACTTTTAAAGTTCTGCTATGTGGCGCGGTATTATCCCGTATTGACGCC
GGGCAAGAGCAACTCGGTCGCCGCATACACTATTCTCAGAATGACTTGGTTGAGT
ACTCACCAGTCACAGAAAAGCATCTTACGGATGGCATGACAGTAAGAGAATTAT
GCAGTGCTGCCATAACCATGAGTGATAACACTGCGGCCAACTTACTTCTGACAAC
GATCGGAGGACCGAAGGAGCTAACCGCTTTTTTGCACAACATGGGGGATCATGT
AACTCGCCTTGATCGTTGGGAACCGGAGCTGAATGAAGCCATACCAAACGACGA
GCGTGACACCACGATGCCTGTAGCAATGGCAACAACGTTGCGCAAACTATTAAC
TGGCGAACTACTTACTCTAGCTTCCCGGCAACAATTAATAGACTGGATGGAGGCG
GATAAAGTTGCAGGACCACTTCTGCGCTCGGCCCTTCCGGCTGGCTGGTTTATTG
CTGATAAATCTGGAGCCGGTGAGCGTGGGTCTCGCGGTATCATTGCAGCACTGGG
GCCAGATGGTAAGCCCTCCCGTATCGTAGTTATCTACACGACGGGGAGTCAGGC
AACTATGGATGAACGAAATAGACAGATCGCTGAGATAGGTGCCTCACTGATTAA
GCATTGGTAACTGTCAGACCAAGTTTACTCATATATACTTTAGATTGATTTAAAA
CTTCATTTTTAATTTAAAAGGATCTAGGTGAAGATCCTTTTTGATAATCTCATGAC
CAAAATCCCTTAACGTGAGTTTTCGTTCCACTGAGCGTCAGACCCCGTAGAAAAG
ATCAAAGGATCTTCTTGAGATCCTTTTTTTCTGCGCGTAATCTGCTGCTTGCAAAC
AAAAAAACCACCGCTACCAGCGGTGGTTTGTTTGCCGGATCAAGAGCTACCAAC
TCTTTTTCCGAAGGTAACTGGCTTCAGCAGAGCGCAGATACCAAATACTGTTCTT
CTAGTGTAGCCGTAGTTAGGCCACCACTTCAAGAACTCTGTAGCACCGCCTACAT
ACCTCGCTCTGCTAATCCTGTTACCAGTGGCTGCTGCCAGTGGCGATAAGTCGTG
TCTTACCGGGTTGGACTCAAGACGATAGTTACCGGATAAGGCGCAGCGGTCGGG
CTGAACGGGGGGTTCGTGCACACAGCCCAGCTTGGAGCGAACGACCTACACCGA
ACTGAGATACCTACAGCGTGAGCTATGAGAAAGCGCCACGCTTCCCGAAGGGAG
AAAGGCGGACAGGTATCCGGTAAGCGGCAGGGTCGGAACAGGAGAGCGCACGA
GGGAGCTTCCAGGGGGAAACGCCTGGTATCTTTATAGTCCTGTCGGGTTTCGCCA
CCTCTGACTTGAGCGTCGATTTTTGTGATGCTCGTCAGGGGGGCGGAGCCTATGG
AAAAACGCCAGCAACGCGGCCTTTTTACGGTTCCTGGCCTTTTGCTGGCCTTTTGC
TCACATGTTCTTTCCTGCGTTATCCCCTGATTCTGTGGATAACCGTATTACCGCCT
TTGAGTGAGCTGATACCGCTCGCCGCAGCCGAACGACCGAGCGCAGCGAGTCAG
TGAGCGAGGAAGCGGAAGAGCGCCCAATACGCAAACCGCCTCTCCCCGCGCGTT
GGCCGATTCATTAATGCAGCTGGCACGACAGGTTTCCCGACTGGAAAGCGGGCA
GTGAGCGCAACGCAATTAATGTGAGTTAGCTCACTCATTAGGCACCCCAGGCTTT
ACACTTTATGCTTCCGGCTCGTATGTTGTGTGGAATTGTGAGCGGATAACAATTT
CACACAGGAAACAGCTATGACCATGATTACGCCAAGCTCTCGAGATCTAGAAAG
CTTCCCGGGGGGATCTG
SEQ ID NO: 16
pscAAV EF1s synMMAB4 5051
GGCCACTCCCTCTCTGCGCGCTCGCTCGCTCACTGAGGCCGGGCGACCTTTGGTC
GCCCGGCCTCAGTGAGCGAGCGAGCGCGCAGAGAGGGAGTGGCCAACTCCATCA
CTAGGGGTTCCTGGAGGGGTGGAGTCGTGACCTAGGGGCTCCGGTGCCCGTCAG
TGGGCAGAGCGCACATCGCCCACAGTCCCCGAGAAGTTGGGGGGAGGGGTCGGC
AATTGAACCGGTGCCTAGAGAAGGTGGCGCGGGGTAAACTGGGAAAGTGATGTC
GTGTACTGGCTCCGCCTTTTTCCCGAGGGTGGGGGAGAACCGTATATAAGTGCAG
TAGTCGCCGTGAACGTTCTTTTTCGCAACGGGTTTGCCGCCAGAACACAGGTCAG
ATCAGATCTTTGTCGATCCTACCATCCACTCGACACACCCGCCAGCGGCCGCGTT
GGTATCAAGGTTACAAGACAGGTTTAAGGAGACCAATAGAAACTGGGCATGTGG
AGACAGAGAAGACTCTTGGGTTTCTGATAGGCACTGACTCTCTTCCTTTGTCCTGT
TCCCATTTCAGAAGCTTCCGAGCTCTCGAATTAAATAAGAGAGAAAAGAAGAGT
AAGAAGAAATATAAGAGCCACCATGGCCGTTTGTGGCCTGGGCTCTAGACTGGG
ACTGGGCTCCCGGCTCGGACTGAGAGGATGTTTTGGCGCCGCTAGACTGCTGTAC
CCCAGATTCCAGAGCCGGGGCCCTCAGGGCGTGGAAGATGGCGATAGACCTCAA
CCTAGCAGCAAGACCCCTAGAATCCCTAAGATCTACACAAAGACCGGCGATAAG
GGCTTCAGCTCCACCTTCACAGGCGAAAGAAGACCTAAGGACGACCAGGTGTTC
GAGGCCGTGGGCACCACCGACGAGCTGAGCTCCGCTATCGGATTTGCCCTGGAG
CTGGTGACAGAGAAGGGCCACACCTTCGCCGAAGAGCTGCAAAAGATTCAGTGC
ACCCTGCAGGACGTGGGCAGCGCCCTGGCCACCCCATGCAGCAGCGCACGGGAA
GCCCACCTGAAGTACACAACCTTTAAGGCCGGACCTATCCTGGAACTGGAACAG
TGGATCGACAAATACACCAGCCAGCTGCCCCCCCTCACAGCCTTCATCCTGCCAA
GCGGCGGCAAGATCAGCTCTGCTCTGCACTTCTGCAGAGCCGTGTGCCGGCGGG
CCGAGAGAAGGGTGGTCCCCCTGGTGCAGATGGGCGAGACAGACGCCAATGTGG
CCAAGTTCCTGAACCGCCTGTCTGATTACCTGTTCACCCTGGCCAGATACGCCGC
CATGAAGGAAGGCAACCAGGAGAAAATCTATATGAAAAACGACCCTTCTGCTGA
GAGCGAGGGGCTGTGAACTCGAGATAACAGGCCTATTGATTGGAAAGTTTGTCA
ACGAATTGTGGGTCTTTTGGGGTTTGCTGCCCCTTTTACGCAATGTGGATATCCTG
CTTTAATGCCTTTATATGCATGTATACAAGCAAAACAGGCTTTTACTTTCTCGCCA
ACTTACAAGGCCTTTCTCAGTAAACAGTATATGACCCTTTACCCCGTTGCTCGGC
AACGGCCTGGTCTGTGCCAAGTGTTTGCTGACGCAACCCCCACTGGTTGGGGCTT
GGCCATAGGCCATCAGCGCATGCGTGGAACCTTTGTGTCTCCTCTGCCGATCCAT
ACTGCGGAACTCCTAGCCGCTTGTTTTGCTCGCAGCAGGTCTGGAGCAAACCTCA
TCGGGACCGACAATTCTGTCGTACTCTCCCGCAAGTATACATCGTTTCCATGGCT
GCTAGGCTGTGCTGCCAACTGGATCCTGCGCGGGACGTCCTTTGTTTACGTCCCG
TCGGCGCTGAATCCCGCGGACGACCCCTCCCGGGGCCGCTTGGGGCTCTACCGCC
CGCTTCTCCGTCTGCCGTACCGTCCGACCACGGGGCGCACCTCTCTTTACGCGGA
CTCCCCGTCTGTGCCTTCTCATCTGCCGGACCGTGTGCACTTCGCTTCACCTCTGC
ACGTCGCATGGAGGCCACCGTGAACGCCCACCGGAACCTGCCCAAGGTCTTGCA
TAAGAGGACTCTTGGACTTTCAGCAATGTCATCTCCGATCTTTTTCCCTCTGCCAA
AAATTATGGGGACATCATGAAGCCCCTTGAGCATCTGACTTCTGGCTAATAAAGG
AAATTTATTTTCATTGCAATAGTGTGTTGGAATTTTTTGTGTCTCTCACACTAGTC
CACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAG
GGACAGATCCGGGCCCGCATGCGTCGACAATTCACTGGCCGTCGTTTTACAACGT
CGTGACTGGGAAAACCCTGGCGTTACCCAACTTAATCGCCTTGCAGCACATCCCC
CTTTCGCCAGCTGGCGTAATAGCGAAGAGGCCCGCACCGATCGCCCTTCCCAACA
GTTGCGCAGCCTGAATGGCGAATGGCGCCTGATGCGGTATTTTCTCCTTACGCAT
CTGTGCGGTATTTCACACCGCATATGGTGCACTCTCAGTACAATCTGCTCTGATG
CCGCATAGTTAAGCCAGCCCCGACACCCGCCAACACCCGCTGACGCGCCCTGAC
GGGCTTGTCTGCTCCCGGCATCCGCTTACAGACAAGCTGTGACCGTCTCCGGGAG
CTGCATGTGTCAGAGGTTTTCACCGTCATCACCGAAACGCGCGAGACGAAAGGG
CCTCGTGATACGCCTATTTTTATAGGTTAATGTCATGATAATAATGGTTTCTTAGA
CGTCAGGTGGCACTTTTCGGGGAAATGTGCGCGGAACCCCTATTTGTTTATTTTTC
TAAATACATTCAAATATGTATCCGCTCATGAGACAATAACCCTGATAAATGCTTC
AATAATATTGAAAAAGGAAGAGTATGAGTATTCAACATTTCCGTGTCGCCCTTAT
TCCCTTTTTTGCGGCATTTTGCCTTCCTGTTTTTGCTCACCCAGAAACGCTGGTGA
AAGTAAAAGATGCTGAAGATCAGTTGGGTGCACGAGTGGGTTACATCGAACTGG
ATCTCAACAGCGGTAAGATCCTTGAGAGTTTTCGCCCCGAAGAACGTTTTCCAAT
GATGAGCACTTTTAAAGTTCTGCTATGTGGCGCGGTATTATCCCGTATTGACGCC
GGGCAAGAGCAACTCGGTCGCCGCATACACTATTCTCAGAATGACTTGGTTGAGT
ACTCACCAGTCACAGAAAAGCATCTTACGGATGGCATGACAGTAAGAGAATTAT
GCAGTGCTGCCATAACCATGAGTGATAACACTGCGGCCAACTTACTTCTGACAAC
GATCGGAGGACCGAAGGAGCTAACCGCTTTTTTGCACAACATGGGGGATCATGT
AACTCGCCTTGATCGTTGGGAACCGGAGCTGAATGAAGCCATACCAAACGACGA
GCGTGACACCACGATGCCTGTAGCAATGGCAACAACGTTGCGCAAACTATTAAC
TGGCGAACTACTTACTCTAGCTTCCCGGCAACAATTAATAGACTGGATGGAGGCG
GATAAAGTTGCAGGACCACTTCTGCGCTCGGCCCTTCCGGCTGGCTGGTTTATTG
CTGATAAATCTGGAGCCGGTGAGCGTGGGTCTCGCGGTATCATTGCAGCACTGGG
GCCAGATGGTAAGCCCTCCCGTATCGTAGTTATCTACACGACGGGGAGTCAGGC
AACTATGGATGAACGAAATAGACAGATCGCTGAGATAGGTGCCTCACTGATTAA
GCATTGGTAACTGTCAGACCAAGTTTACTCATATATACTTTAGATTGATTTAAAA
CTTCATTTTTAATTTAAAAGGATCTAGGTGAAGATCCTTTTTGATAATCTCATGAC
CAAAATCCCTTAACGTGAGTTTTCGTTCCACTGAGCGTCAGACCCCGTAGAAAAG
ATCAAAGGATCTTCTTGAGATCCTTTTTTTCTGCGCGTAATCTGCTGCTTGCAAAC
AAAAAAACCACCGCTACCAGCGGTGGTTTGTTTGCCGGATCAAGAGCTACCAAC
TCTTTTTCCGAAGGTAACTGGCTTCAGCAGAGCGCAGATACCAAATACTGTTCTT
CTAGTGTAGCCGTAGTTAGGCCACCACTTCAAGAACTCTGTAGCACCGCCTACAT
ACCTCGCTCTGCTAATCCTGTTACCAGTGGCTGCTGCCAGTGGCGATAAGTCGTG
TCTTACCGGGTTGGACTCAAGACGATAGTTACCGGATAAGGCGCAGCGGTCGGG
CTGAACGGGGGGTTCGTGCACACAGCCCAGCTTGGAGCGAACGACCTACACCGA
ACTGAGATACCTACAGCGTGAGCTATGAGAAAGCGCCACGCTTCCCGAAGGGAG
AAAGGCGGACAGGTATCCGGTAAGCGGCAGGGTCGGAACAGGAGAGCGCACGA
GGGAGCTTCCAGGGGGAAACGCCTGGTATCTTTATAGTCCTGTCGGGTTTCGCCA
CCTCTGACTTGAGCGTCGATTTTTGTGATGCTCGTCAGGGGGGCGGAGCCTATGG
AAAAACGCCAGCAACGCGGCCTTTTTACGGTTCCTGGCCTTTTGCTGGCCTTTTGC
TCACATGTTCTTTCCTGCGTTATCCCCTGATTCTGTGGATAACCGTATTACCGCCT
TTGAGTGAGCTGATACCGCTCGCCGCAGCCGAACGACCGAGCGCAGCGAGTCAG
TGAGCGAGGAAGCGGAAGAGCGCCCAATACGCAAACCGCCTCTCCCCGCGCGTT
GGCCGATTCATTAATGCAGCTGGCACGACAGGTTTCCCGACTGGAAAGCGGGCA
GTGAGCGCAACGCAATTAATGTGAGTTAGCTCACTCATTAGGCACCCCAGGCTTT
ACACTTTATGCTTCCGGCTCGTATGTTGTGTGGAATTGTGAGCGGATAACAATTT
CACACAGGAAACAGCTATGACCATGATTACGCCAAGCTCTCGAGATCTAGAAAG
CTTCCCGGGGGGATCTG
SEQ ID NO: 17
pscAAV EF1s synMMAB5 5051
GGCCACTCCCTCTCTGCGCGCTCGCTCGCTCACTGAGGCCGGGCGACCAAAGGTC
GCCCGGCCTCAGTGAGCGAGCGAGCGCGCAGAGAGGGAGTGGCCAACTCCATCA
CTAGGGGTTCCTGGAGGGGTGGAGTCGTGACCTAGGGGCTCCGGTGCCCGTCAG
TGGGCAGAGCGCACATCGCCCACAGTCCCCGAGAAGTTGGGGGGAGGGGTCGGC
AATTGAACCGGTGCCTAGAGAAGGTGGCGCGGGGTAAACTGGGAAAGTGATGTC
GTGTACTGGCTCCGCCTTTTTCCCGAGGGTGGGGGAGAACCGTATATAAGTGCAG
TAGTCGCCGTGAACGTTCTTTTTCGCAACGGGTTTGCCGCCAGAACACAGGTCAG
ATCAGATCTTTGTCGATCCTACCATCCACTCGACACACCCGCCAGCGGCCGCGTT
GGTATCAAGGTTACAAGACAGGTTTAAGGAGACCAATAGAAACTGGGCATGTGG
AGACAGAGAAGACTCTTGGGTTTCTGATAGGCACTGACTCTCTTCCTTTGTCCTGT
TCCCATTTCAGAAGCTTCCGAGCTCTCGAATTAAATAAGAGAGAAAAGAAGAGT
AAGAAGAAATATAAGAGCCACCATGGCCGTGTGTGGACTGGGCAGCAGACTGGG
ACTGGGATCTAGACTGGGACTGAGAGGCTGCTTCGGCGCTGCTAGACTGCTGTAC
CCTAGATTCCAGTCCAGAGGACCCCAAGGCGTCGAGGATGGAGATAGACCCCAG
CCCTCCTCCAAGACCCCTAGAATCCCCAAGATCTACACCAAGACCGGCGACAAG
GGCTTCAGCTCCACCTTTACCGGCGAGAGGAGACCCAAGGACGATCAAGTGTTC
GAGGCCGTGGGAACCACCGACGAGCTGAGCTCCGCTATCGGCTTCGCTCTGGAG
CTGGTGACAGAGAAGGGCCACACCTTTGCCGAGGAGCTGCAGAAGATCCAGTGC
ACACTGCAAGACGTGGGCAGCGCTCTGGCTACACCTTGCAGCAGCGCTAGAGAG
GCCCATCTGAAATACACCACCTTCAAAGCCGGCCCTATTCTGGAGCTGGAACAGT
GGATCGACAAGTACACCAGCCAGCTGCCTCCTCTGACAGCCTTCATTCTGCCCAG
CGGCGGAAAGATCTCCAGCGCTCTGCACTTCTGCAGAGCCGTCTGCAGAAGGGC
TGAGAGAAGGGTGGTGCCTCTGGTGCAGATGGGCGAGACCGATGCCAACGTGGC
CAAGTTCCTCAATAGACTGTCCGATTATCTGTTCACACTGGCTAGATATGCCGCC
ATGAAGGAGGGCAACCAAGAGAAGATCTACATGAAGAACGATCCCAGCGCCGA
GAGCGAGGGCCTCTGAACTCGAGATAACAGGCCTATTGATTGGAAAGTTTGTCA
ACGAATTGTGGGTCTTTTGGGGTTTGCTGCCCCTTTTACGCAATGTGGATATCCTG
CTTTAATGCCTTTATATGCATGTATACAAGCAAAACAGGCTTTTACTTTCTCGCCA
ACTTACAAGGCCTTTCTCAGTAAACAGTATATGACCCTTTACCCCGTTGCTCGGC
AACGGCCTGGTCTGTGCCAAGTGTTTGCTGACGCAACCCCCACTGGTTGGGGCTT
GGCCATAGGCCATCAGCGCATGCGTGGAACCTTTGTGTCTCCTCTGCCGATCCAT
ACTGCGGAACTCCTAGCCGCTTGTTTTGCTCGCAGCAGGTCTGGAGCAAACCTCA
TCGGGACCGACAATTCTGTCGTACTCTCCCGCAAGTATACATCGTTTCCATGGCT
GCTAGGCTGTGCTGCCAACTGGATCCTGCGCGGGACGTCCTTTGTTTACGTCCCG
TCGGCGCTGAATCCCGCGGACGACCCCTCCCGGGGCCGCTTGGGGCTCTACCGCC
CGCTTCTCCGTCTGCCGTACCGTCCGACCACGGGGCGCACCTCTCTTTACGCGGA
CTCCCCGTCTGTGCCTTCTCATCTGCCGGACCGTGTGCACTTCGCTTCACCTCTGC
ACGTCGCATGGAGGCCACCGTGAACGCCCACCGGAACCTGCCCAAGGTCTTGCA
TAAGAGGACTCTTGGACTTTCAGCAATGTCATCTCCGATCTTTTTCCCTCTGCCAA
AAATTATGGGGACATCATGAAGCCCCTTGAGCATCTGACTTCTGGCTAATAAAGG
AAATTTATTTTCATTGCAATAGTGTGTTGGAATTTTTTGTGTCTCTCACACTAGTC
CACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGAGAG
GGACAGATCCGGGCCCGCATGCGTCGACAATTCACTGGCCGTCGTTTTACAACGT
CGTGACTGGGAAAACCCTGGCGTTACCCAACTTAATCGCCTTGCAGCACATCCCC
CTTTCGCCAGCTGGCGTAATAGCGAAGAGGCCCGCACCGATCGCCCTTCCCAACA
GTTGCGCAGCCTGAATGGCGAATGGCGCCTGATGCGGTATTTTCTCCTTACGCAT
CTGTGCGGTATTTCACACCGCATATGGTGCACTCTCAGTACAATCTGCTCTGATG
CCGCATAGTTAAGCCAGCCCCGACACCCGCCAACACCCGCTGACGCGCCCTGAC
GGGCTTGTCTGCTCCCGGCATCCGCTTACAGACAAGCTGTGACCGTCTCCGGGAG
CTGCATGTGTCAGAGGTTTTCACCGTCATCACCGAAACGCGCGAGACGAAAGGG
CCTCGTGATACGCCTATTTTTATAGGTTAATGTCATGATAATAATGGTTTCTTAGA
CGTCAGGTGGCACTTTTCGGGGAAATGTGCGCGGAACCCCTATTTGTTTATTTTTC
TAAATACATTCAAATATGTATCCGCTCATGAGACAATAACCCTGATAAATGCTTC
AATAATATTGAAAAAGGAAGAGTATGAGTATTCAACATTTCCGTGTCGCCCTTAT
TCCCTTTTTTGCGGCATTTTGCCTTCCTGTTTTTGCTCACCCAGAAACGCTGGTGA
AAGTAAAAGATGCTGAAGATCAGTTGGGTGCACGAGTGGGTTACATCGAACTGG
ATCTCAACAGCGGTAAGATCCTTGAGAGTTTTCGCCCCGAAGAACGTTTTCCAAT
GATGAGCACTTTTAAAGTTCTGCTATGTGGCGCGGTATTATCCCGTATTGACGCC
GGGCAAGAGCAACTCGGTCGCCGCATACACTATTCTCAGAATGACTTGGTTGAGT
ACTCACCAGTCACAGAAAAGCATCTTACGGATGGCATGACAGTAAGAGAATTAT
GCAGTGCTGCCATAACCATGAGTGATAACACTGCGGCCAACTTACTTCTGACAAC
GATCGGAGGACCGAAGGAGCTAACCGCTTTTTTGCACAACATGGGGGATCATGT
AACTCGCCTTGATCGTTGGGAACCGGAGCTGAATGAAGCCATACCAAACGACGA
GCGTGACACCACGATGCCTGTAGCAATGGCAACAACGTTGCGCAAACTATTAAC
TGGCGAACTACTTACTCTAGCTTCCCGGCAACAATTAATAGACTGGATGGAGGCG
GATAAAGTTGCAGGACCACTTCTGCGCTCGGCCCTTCCGGCTGGCTGGTTTATTG
CTGATAAATCTGGAGCCGGTGAGCGTGGGTCTCGCGGTATCATTGCAGCACTGGG
GCCAGATGGTAAGCCCTCCCGTATCGTAGTTATCTACACGACGGGGAGTCAGGC
AACTATGGATGAACGAAATAGACAGATCGCTGAGATAGGTGCCTCACTGATTAA
GCATTGGTAACTGTCAGACCAAGTTTACTCATATATACTTTAGATTGATTTAAAA
CTTCATTTTTAATTTAAAAGGATCTAGGTGAAGATCCTTTTTGATAATCTCATGAC
CAAAATCCCTTAACGTGAGTTTTCGTTCCACTGAGCGTCAGACCCCGTAGAAAAG
ATCAAAGGATCTTCTTGAGATCCTTTTTTTCTGCGCGTAATCTGCTGCTTGCAAAC
AAAAAAACCACCGCTACCAGCGGTGGTTTGTTTGCCGGATCAAGAGCTACCAAC
TCTTTTTCCGAAGGTAACTGGCTTCAGCAGAGCGCAGATACCAAATACTGTTCTT
CTAGTGTAGCCGTAGTTAGGCCACCACTTCAAGAACTCTGTAGCACCGCCTACAT
ACCTCGCTCTGCTAATCCTGTTACCAGTGGCTGCTGCCAGTGGCGATAAGTCGTG
TCTTACCGGGTTGGACTCAAGACGATAGTTACCGGATAAGGCGCAGCGGTCGGG
CTGAACGGGGGGTTCGTGCACACAGCCCAGCTTGGAGCGAACGACCTACACCGA
ACTGAGATACCTACAGCGTGAGCTATGAGAAAGCGCCACGCTTCCCGAAGGGAG
AAAGGCGGACAGGTATCCGGTAAGCGGCAGGGTCGGAACAGGAGAGCGCACGA
GGGAGCTTCCAGGGGGAAACGCCTGGTATCTTTATAGTCCTGTCGGGTTTCGCCA
CCTCTGACTTGAGCGTCGATTTTTGTGATGCTCGTCAGGGGGGCGGAGCCTATGG
AAAAACGCCAGCAACGCGGCCTTTTTACGGTTCCTGGCCTTTTGCTGGCCTTTTGC
TCACATGTTCTTTCCTGCGTTATCCCCTGATTCTGTGGATAACCGTATTACCGCCT
TTGAGTGAGCTGATACCGCTCGCCGCAGCCGAACGACCGAGCGCAGCGAGTCAG
TGAGCGAGGAAGCGGAAGAGCGCCCAATACGCAAACCGCCTCTCCCCGCGCGTT
GGCCGATTCATTAATGCAGCTGGCACGACAGGTTTCCCGACTGGAAAGCGGGCA
GTGAGCGCAACGCAATTAATGTGAGTTAGCTCACTCATTAGGCACCCCAGGCTTT
ACACTTTATGCTTCCGGCTCGTATGTTGTGTGGAATTGTGAGCGGATAACAATTT
CACACAGGAAACAGCTATGACCATGATTACGCCAAGCTCTCGAGATCTAGAAAG
CTTCCCGGGGGGATCTG
SEQ ID NO: 18
pscAAV EF1s synMMAB6 5051
GGCCACTCCCTCTCTGCGCGCTCGCTCGCTCACTGAGGCCGGGCGACCAAAGGTC
GCCCGGCCTCAGTGAGCGAGCGAGCGCGCAGAGAGGGAGTGGCCAACTCCATCA
CTAGGGGTTCCTGGAGGGGTGGAGTCGTGACCTAGGGGCTCCGGTGCCCGTCAG
TGGGCAGAGCGCACATCGCCCACAGTCCCCGAGAAGTTGGGGGGAGGGGTCGGC
AATTGAACCGGTGCCTAGAGAAGGTGGCGCGGGGTAAACTGGGAAAGTGATGTC
GTGTACTGGCTCCGCCTTTTTCCCGAGGGTGGGGGAGAACCGTATATAAGTGCAG
TAGTCGCCGTGAACGTTCTTTTTCGCAACGGGTTTGCCGCCAGAACACAGGTCAG
ATCAGATCTTTGTCGATCCTACCATCCACTCGACACACCCGCCAGCGGCCGCGTT
GGTATCAAGGTTACAAGACAGGTTTAAGGAGACCAATAGAAACTGGGCATGTGG
AGACAGAGAAGACTCTTGGGTTTCTGATAGGCACTGACTCTCTTCCTTTGTCCTGT
TCCCATTTCAGAAGCTTCCGAGCTCTCGAATTAAATAAGAGAGAAAAGAAGAGT
AAGAAGAAATATAAGAGCCACCATGGCCGTGTGTGGACTGGGCAGCAGACTGGG
ACTGGGCAGCAGACTGGGACTGAGAGGATGCTTTGGCGCCGCTAGACTGCTGTA
CCCTAGATTCCAGTCTAGAGGCCCTCAAGGCGTGGAGGATGGAGACAGACCCCA
GCCCAGCTCCAAAACCCCTAGAATCCCCAAGATCTACACCAAGACCGGCGACAA
GGGCTTCAGCAGCACCTTCACCGGCGAGAGAAGACCCAAGGATGACCAAGTGTT
CGAGGCCGTGGGCACCACCGACGAACTGTCCAGCGCCATTGGCTTCGCTCTGGA
GCTGGTGACCGAGAAGGGACACACCTTTGCCGAGGAGCTGCAGAAGATTCAGTG
CACACTGCAAGACGTGGGCAGCGCTCTGGCCACCCCTTGCAGCTCCGCTAGGGA
AGCCCATCTGAAGTACACCACCTTCAAGGCCGGCCCTATTCTGGAGCTCGAGCAG
TGGATCGACAAATACACCAGCCAGCTGCCTCCTCTGACCGCCTTCATTCTGCCTT
CCGGAGGCAAGATCTCCAGCGCTCTGCACTTCTGTAGAGCCGTGTGTAGAAGAG
CCGAAAGAAGAGTGGTCCCTCTGGTGCAGATGGGAGAGACCGACGCCAACGTGG
CCAAGTTTCTGAATAGACTGAGCGATTATCTGTTCACACTGGCTAGATACGCCGC
CATGAAGGAGGGCAACCAAGAGAAGATCTACATGAAGAACGACCCCTCCGCCG
AGAGCGAAGGACTGTGAACTCGAGATAACAGGCCTATTGATTGGAAAGTTTGTC
AACGAATTGTGGGTCTTTTGGGGTTTGCTGCCCCTTTTACGCAATGTGGATATCCT
GCTTTAATGCCTTTATATGCATGTATACAAGCAAAACAGGCTTTTACTTTCTCGCC
AACTTACAAGGCCTTTCTCAGTAAACAGTATATGACCCTTTACCCCGTTGCTCGG
CAACGGCCTGGTCTGTGCCAAGTGTTTGCTGACGCAACCCCCACTGGTTGGGGCT
TGGCCATAGGCCATCAGCGCATGCGTGGAACCTTTGTGTCTCCTCTGCCGATCCA
TACTGCGGAACTCCTAGCCGCTTGTTTTGCTCGCAGCAGGTCTGGAGCAAACCTC
ATCGGGACCGACAATTCTGTCGTACTCTCCCGCAAGTATACATCGTTTCCATGGC
TGCTAGGCTGTGCTGCCAACTGGATCCTGCGCGGGACGTCCTTTGTTTACGTCCC
GTCGGCGCTGAATCCCGCGGACGACCCCTCCCGGGGCCGCTTGGGGCTCTACCGC
CCGCTTCTCCGTCTGCCGTACCGTCCGACCACGGGGCGCACCTCTCTTTACGCGG
ACTCCCCGTCTGTGCCTTCTCATCTGCCGGACCGTGTGCACTTCGCTTCACCTCTG
CACGTCGCATGGAGGCCACCGTGAACGCCCACCGGAACCTGCCCAAGGTCTTGC
ATAAGAGGACTCTTGGACTTTCAGCAATGTCATCTCCGATCTTTTTCCCTCTGCCA
AAAATTATGGGGACATCATGAAGCCCCTTGAGCATCTGACTTCTGGCTAATAAAG
GAAATTTATTTTCATTGCAATAGTGTGTTGGAATTTTTTGTGTCTCTCACACTAGT
CCACTCCCTCTCTGCGCGCTCGCTCGCTCACTGAGGCCGGGCGACCAAAGGTCGC
CCGACGCCCGGGCTTTGCCCGGGCGGCCTCAGTGAGCGAGCGAGCGCGCAGAGA
GGGACAGATCCGGGCCCGCATGCGTCGACAATTCACTGGCCGTCGTTTTACAACG
TCGTGACTGGGAAAACCCTGGCGTTACCCAACTTAATCGCCTTGCAGCACATCCC
CCTTTCGCCAGCTGGCGTAATAGCGAAGAGGCCCGCACCGATCGCCCTTCCCAAC
AGTTGCGCAGCCTGAATGGCGAATGGCGCCTGATGCGGTATTTTCTCCTTACGCA
TCTGTGCGGTATTTCACACCGCATATGGTGCACTCTCAGTACAATCTGCTCTGATG
CCGCATAGTTAAGCCAGCCCCGACACCCGCCAACACCCGCTGACGCGCCCTGAC
GGGCTTGTCTGCTCCCGGCATCCGCTTACAGACAAGCTGTGACCGTCTCCGGGAG
CTGCATGTGTCAGAGGTTTTCACCGTCATCACCGAAACGCGCGAGACGAAAGGG
CCTCGTGATACGCCTATTTTTATAGGTTAATGTCATGATAATAATGGTTTCTTAGA
CGTCAGGTGGCACTTTTCGGGGAAATGTGCGCGGAACCCCTATTTGTTTATTTTTC
TAAATACATTCAAATATGTATCCGCTCATGAGACAATAACCCTGATAAATGCTTC
AATAATATTGAAAAAGGAAGAGTATGAGTATTCAACATTTCCGTGTCGCCCTTAT
TCCCTTTTTTGCGGCATTTTGCCTTCCTGTTTTTGCTCACCCAGAAACGCTGGTGA
AAGTAAAAGATGCTGAAGATCAGTTGGGTGCACGAGTGGGTTACATCGAACTGG
ATCTCAACAGCGGTAAGATCCTTGAGAGTTTTCGCCCCGAAGAACGTTTTCCAAT
GATGAGCACTTTTAAAGTTCTGCTATGTGGCGCGGTATTATCCCGTATTGACGCC
GGGCAAGAGCAACTCGGTCGCCGCATACACTATTCTCAGAATGACTTGGTTGAGT
ACTCACCAGTCACAGAAAAGCATCTTACGGATGGCATGACAGTAAGAGAATTAT
GCAGTGCTGCCATAACCATGAGTGATAACACTGCGGCCAACTTACTTCTGACAAC
GATCGGAGGACCGAAGGAGCTAACCGCTTTTTTGCACAACATGGGGGATCATGT
AACTCGCCTTGATCGTTGGGAACCGGAGCTGAATGAAGCCATACCAAACGACGA
GCGTGACACCACGATGCCTGTAGCAATGGCAACAACGTTGCGCAAACTATTAAC
TGGCGAACTACTTACTCTAGCTTCCCGGCAACAATTAATAGACTGGATGGAGGCG
GATAAAGTTGCAGGACCACTTCTGCGCTCGGCCCTTCCGGCTGGCTGGTTTATTG
CTGATAAATCTGGAGCCGGTGAGCGTGGGTCTCGCGGTATCATTGCAGCACTGGG
GCCAGATGGTAAGCCCTCCCGTATCGTAGTTATCTACACGACGGGGAGTCAGGC
AACTATGGATGAACGAAATAGACAGATCGCTGAGATAGGTGCCTCACTGATTAA
GCATTGGTAACTGTCAGACCAAGTTTACTCATATATACTTTAGATTGATTTAAAA
CTTCATTTTTAATTTAAAAGGATCTAGGTGAAGATCCTTTTTGATAATCTCATGAC
CAAAATCCCTTAACGTGAGTTTTCGTTCCACTGAGCGTCAGACCCCGTAGAAAAG
ATCAAAGGATCTTCTTGAGATCCTTTTTTTCTGCGCGTAATCTGCTGCTTGCAAAC
AAAAAAACCACCGCTACCAGCGGTGGTTTGTTTGCCGGATCAAGAGCTACCAAC
TCTTTTTCCGAAGGTAACTGGCTTCAGCAGAGCGCAGATACCAAATACTGTTCTT
CTAGTGTAGCCGTAGTTAGGCCACCACTTCAAGAACTCTGTAGCACCGCCTACAT
ACCTCGCTCTGCTAATCCTGTTACCAGTGGCTGCTGCCAGTGGCGATAAGTCGTG
TCTTACCGGGTTGGACTCAAGACGATAGTTACCGGATAAGGCGCAGCGGTCGGG
CTGAACGGGGGGTTCGTGCACACAGCCCAGCTTGGAGCGAACGACCTACACCGA
ACTGAGATACCTACAGCGTGAGCTATGAGAAAGCGCCACGCTTCCCGAAGGGAG
AAAGGCGGACAGGTATCCGGTAAGCGGCAGGGTCGGAACAGGAGAGCGCACGA
GGGAGCTTCCAGGGGGAAACGCCTGGTATCTTTATAGTCCTGTCGGGTTTCGCCA
CCTCTGACTTGAGCGTCGATTTTTGTGATGCTCGTCAGGGGGGCGGAGCCTATGG
AAAAACGCCAGCAACGCGGCCTTTTTACGGTTCCTGGCCTTTTGCTGGCCTTTTGC
TCACATGTTCTTTCCTGCGTTATCCCCTGATTCTGTGGATAACCGTATTACCGCCT
TTGAGTGAGCTGATACCGCTCGCCGCAGCCGAACGACCGAGCGCAGCGAGTCAG
TGAGCGAGGAAGCGGAAGAGCGCCCAATACGCAAACCGCCTCTCCCCGCGCGTT
GGCCGATTCATTAATGCAGCTGGCACGACAGGTTTCCCGACTGGAAAGCGGGCA
GTGAGCGCAACGCAATTAATGTGAGTTAGCTCACTCATTAGGCACCCCAGGCTTT
ACACTTTATGCTTCCGGCTCGTATGTTGTGTGGAATTGTGAGCGGATAACAATTT
CACACAGGAAACAGCTATGACCATGATTACGCCAAGCTCTCGAGATCTAGAAAG
CTTCCCGGGGGGATCTG

TABLE 5

| | | | | plasmid | |
|---|---|---|---|---|---|
| Plasmid | | Config- | Poly- | size | |
| Number | Plasmid | uration | nucleotide | (bp) | copies/μg |
| 1 | pAAV EF1L | SS | WT | 6124 | 1.591E+11 |
| | MMAB WT | | | | |
| 2 | pAAV EF1L | SS | codon | 6112 | 1.594E+11 |
| | synMMAB1 | | optimized | | |
| 3 | pAA VEF1L | SS | codon | 6112 | 1.594E+11 |
| | synMMAB2 | | optimized | | |
| 4 | pscAAV EF1s | SC | WT | 5216 | 1.868E+11 |
| | MMAB WT BGH | | | | |
| 5 | pscAAV EF1s | SC | WT | 5051 | 1.929E+11 |
| | MMAB WT RBG | | | | |
| 6 | pscAAV EF1s | SC | codon | 5051 | 1.929E+11 |
| | synMMAB1 | | optimized | | |
| 7 | pscAAV EF1s | SC | codon | 5019 | 1.942E+11 |
| | synMMAB2 | | optimized | | |
| 8 | pscAAV EF1s | SC | codon | 5051 | 1.929E+11 |
| | synMMAB3 | | optimized | | |
| 9 | pscAAV EF1s | SC | codon | 5051 | 1.929E+11 |
| | synMMAB4 | | optimized | | |
| 10a | pscAAV EF1s | SC | codon | 5051 | 1.929E+11 |
| | synMMAB5 | | optimized | | |
| 10b | pscAAV EF1s | SC | codon | 5051 | 1.929E+11 |
| | synMMAB5 | | optimized | | |
| 11 | pscAAV EF1s | SC | codon | 5051 | 1.929E+11 |
| | synMMAB6 | | optimized | | |

### Example 4

This example demonstrates the efficacy of the AAV synMMAB vectors at driving expression of MMAB in HEK293 MMAB KO cells.

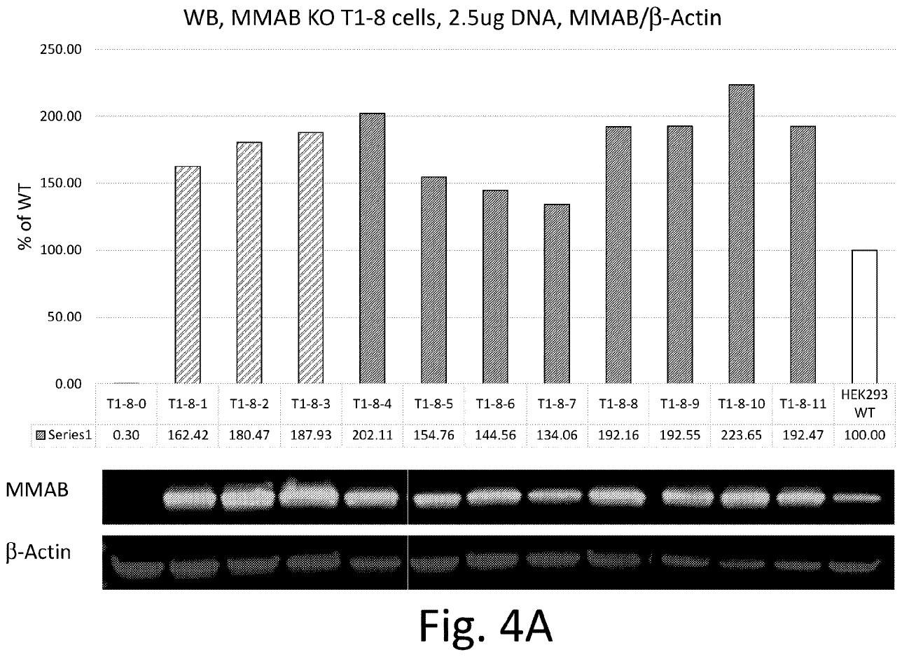

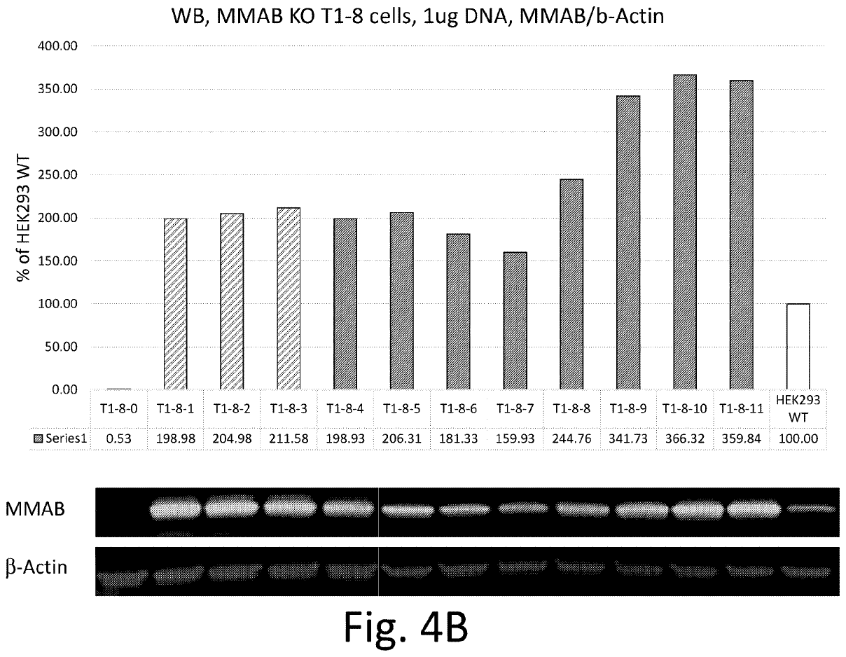

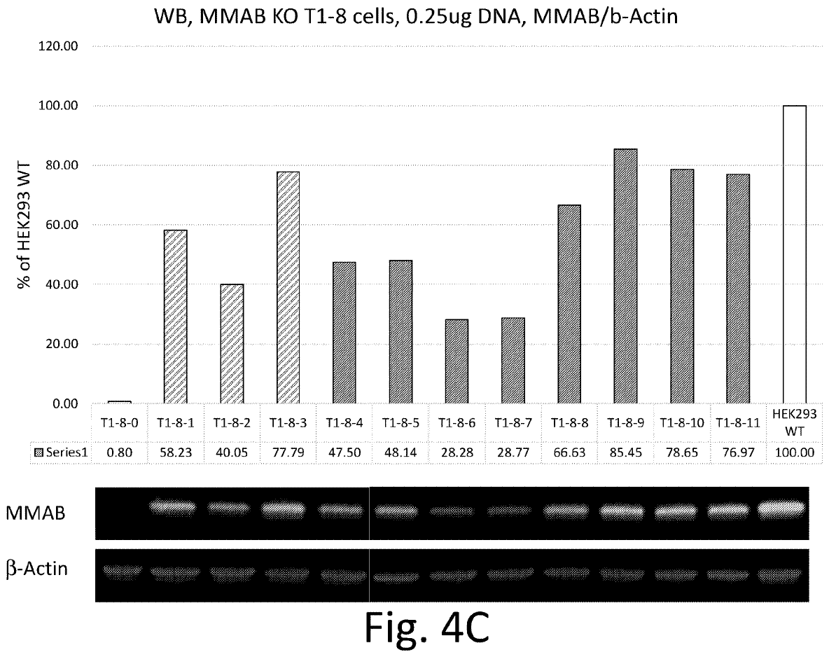

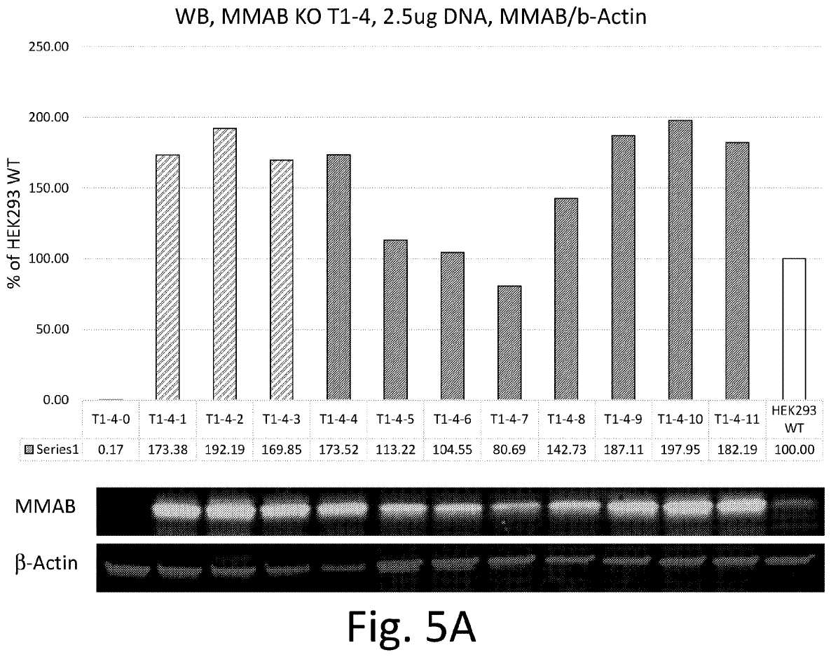

To test the efficacy of plasmids that contain the AAV backbone designed to express synMMAB1 and synMMAB2 transgenes in the single-stranded (ss) configuration, they were transfected into HEK293 cells and tested for expression. As assessed by western blotting, pAAV EF1L synMMAB2 displayed superior expression compared to synMMAB1 or the wild type MMAB sequence at DNA concentrations of 0.5 μg (FIG. 4C), 1 μg (FIG. 4B), and 2.5 μg (FIG. 4C).

Similarly, to test the efficacy of plasmids that contain the AAV backbone designed to express synMMAB1-synMMAB6 AAV transgenes in the self-complementary (sc) configuration, they were transfected into HEK293 cells and tested for expression. As assessed by western blotting, these studies showed that pscAAV EF1s synMMAB5 displayed superior expression compared to other synMMAB polynucleotides or the wild type MMAB sequence across the DNA concentrations of 0.5 μg (FIG. 4C), 1 μg (FIG. 4B), and 2.5 μg (FIG. 4A).

Then western blotting for MMAB expression was performed as previously described on HEK293 MMAB WT cells, HEK 293 MMAB KO T1-8 cells (T1-8-0), and HEK 293 MMAB KO T1-8 cells that were transfected with pAAV EF1L MMAB WT plasmid (T1-8-1), pAAV EF1L synMMAB1 (T1-8-2), pAAV EF1L synMMAB2 plasmid (T1-8-3), pscAAV EF1s MMAB WT BGH plasmid (T1-8-4), pscAAV EF1s MMAB WT RGB plasmid (T1-8-5), pscAAV EF1s synMMAB3 plasmid (T1-8-6), pscAAV EF1s synMMAB2 plasmid (T1-8-7), pscAAV EF1s synMMAB3 plasmid (T1-8-8), pscAAV EF1s synMMAB4 plasmid (T1-8-9), pscAAV EF1s synMMAB5 plasmid (T1-8-10), and pscAAV EF1s synMMAB6 plasmid (T1-8-11) when using 2.5 μg of plasmid (FIG. 4A), 1 μg of plasmid (FIG. 4B), and 0.25 μg of plasmid (FIG. 4C).

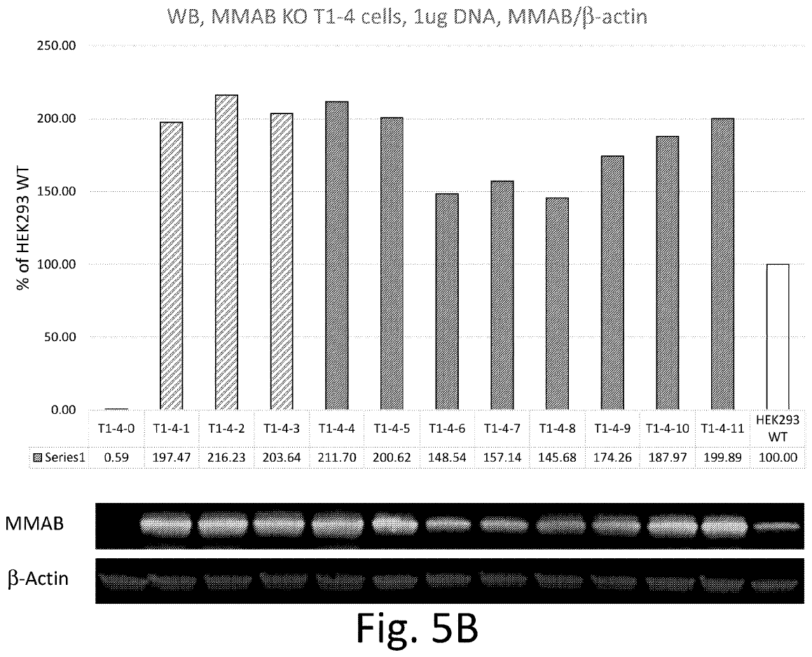

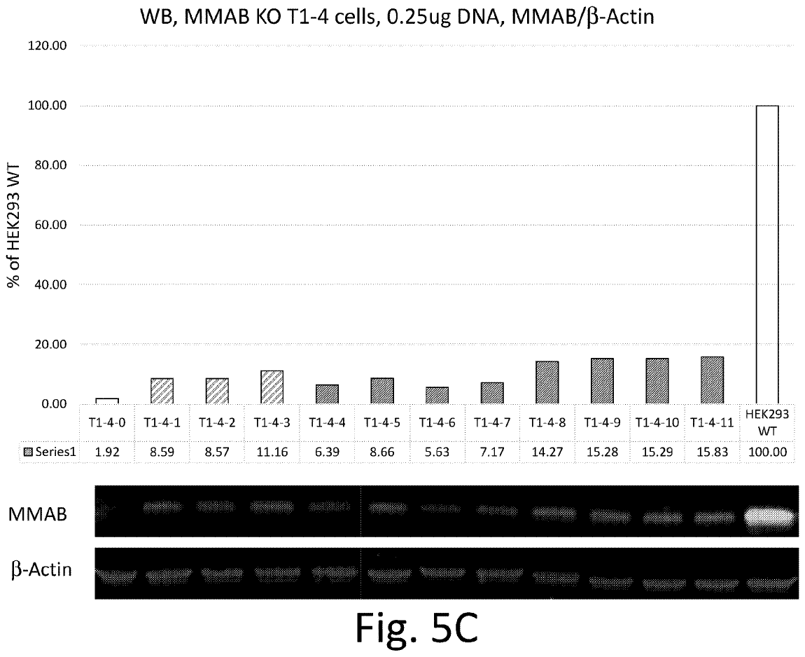

Western blotting for MMAB expression was also performed on HEK293 MMAB WT cells (T1-5), HEK 293 MMAB KO T1-4 cells (T1-4-0), and HEK 293 MMAB KO T1-4 cells that were transfected with pAAV EF1 LMMAB WT plasmid (T1-4-1), pAAV EF1L synMMAB1 (T1-4-2), pAAV EF1L synMMAB2 plasmid (T1-4-3), pscAAV EF1sA AB WT BGH plasmid (T1-4-4), pscAAV EF1s MMAB WT RGB plasmid (T1-4-5), pscAAV EF1s synMMAB1 plasmid (T1-4-6), pscAAV EF1s synMAAB2 plasmid (T1-4-7), pscAAV EF1s synMMAB3 plasmid (T1-4-8), pscAAV EF1s synMMAB4 plasmid (T1-4-9), pscAAV EF1s synMMAB5 plasmid (T1-4-10), and pscAAV EF1s synMMAB6 plasmid (T1-4-11), when using 2.5 μg of plasmid (FIG. 5A), 1 μg of plasmid (FIG. 5B), and 0.25 μg of plasmid (FIG. 5C).

These results suggest that pAAV EF1L synMMAB2 and pAAV EF1s synMMAB5 are the most consistently effective drivers of MMAB expression.

### Example 5

This example demonstrates the efficacy of the AAV synMMAB vectors pseudoserotyped with AAV8 or AAV9 capsids at driving expression of MMAB in HEK293 MMAB KO cells.

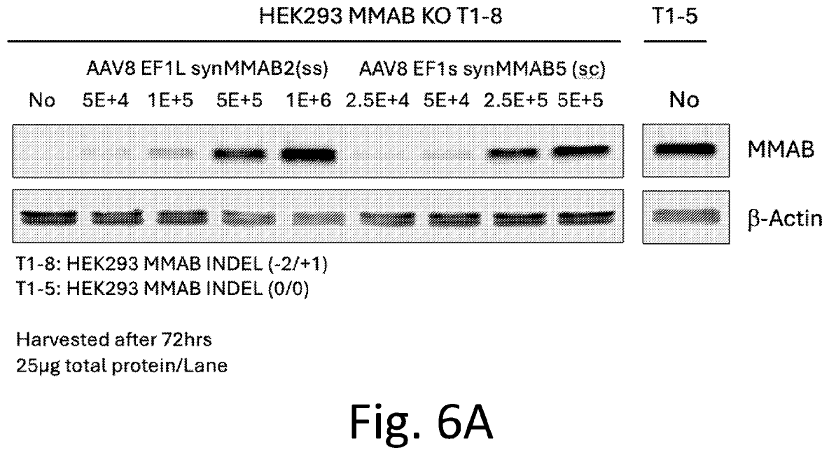

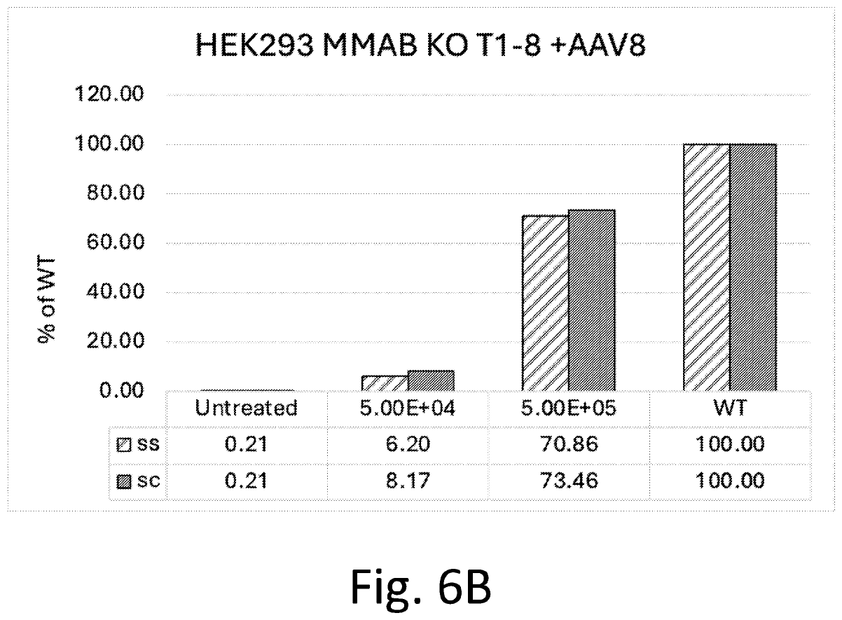

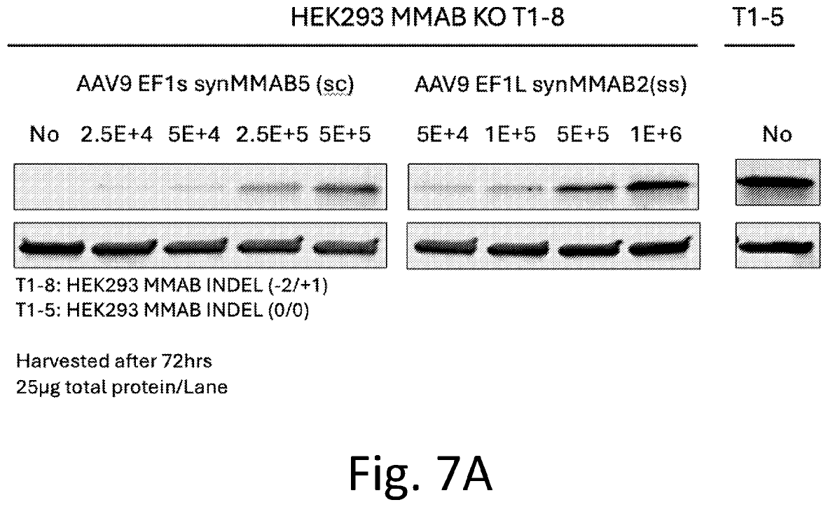

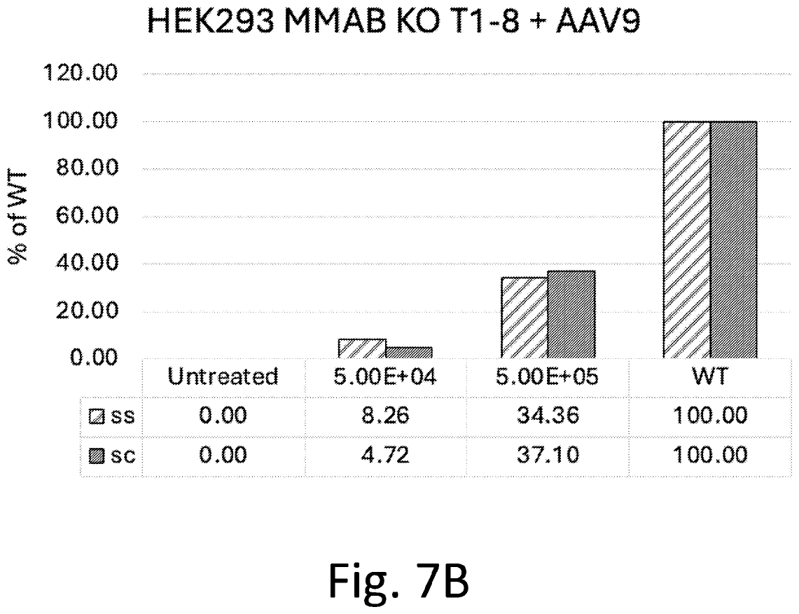

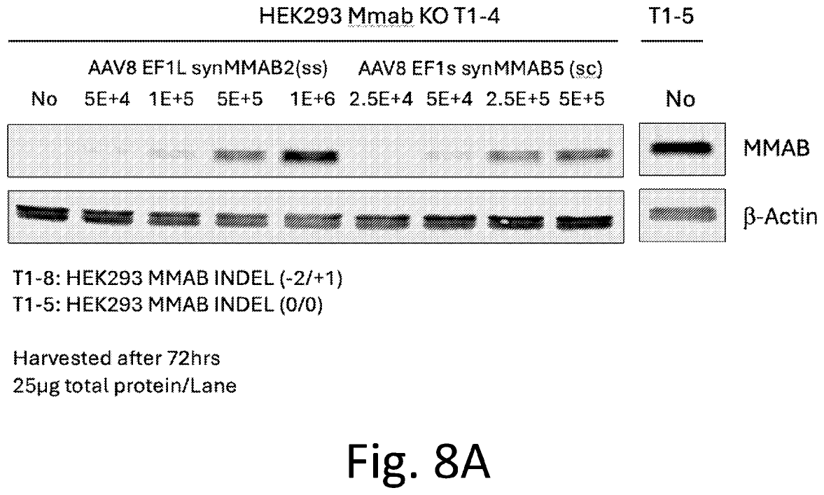

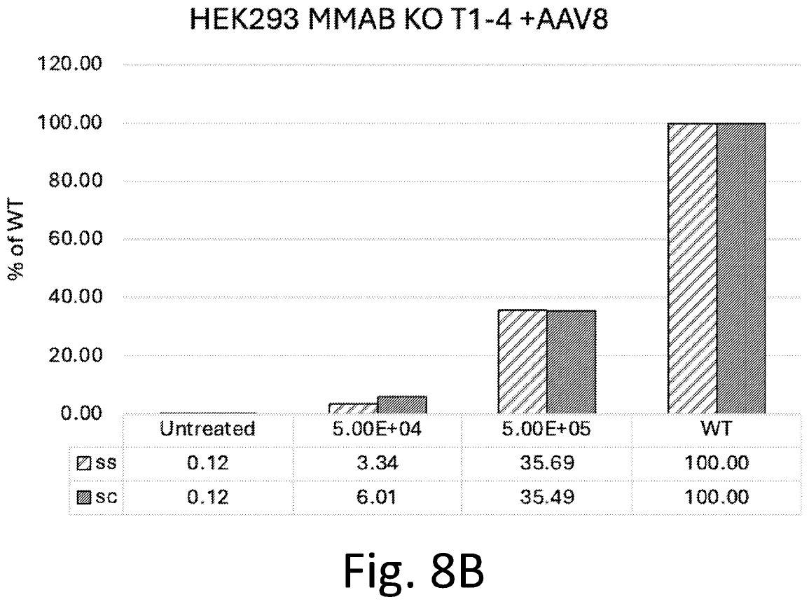

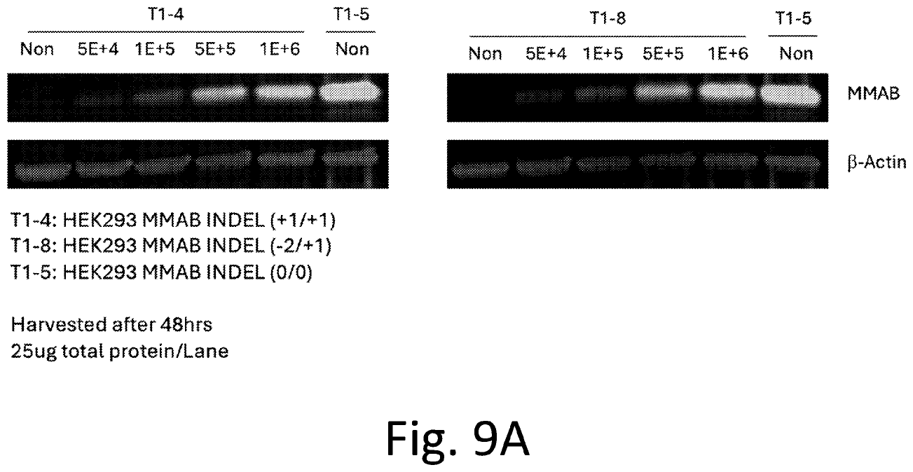

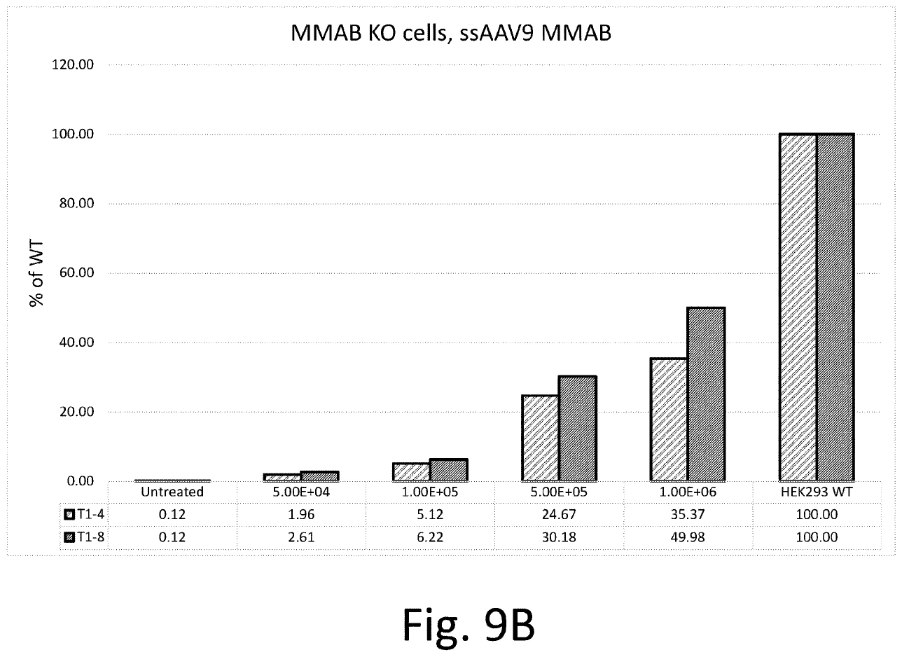

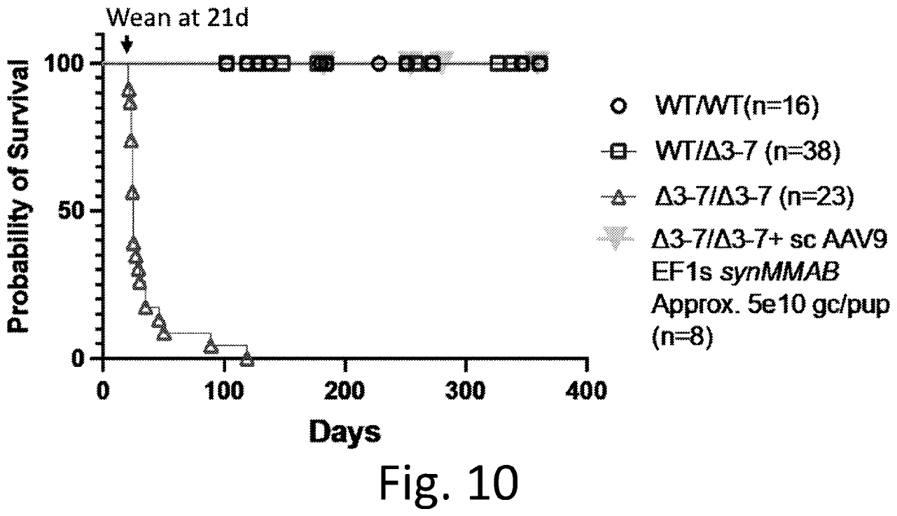

The lead AAV plasmids, packaged as either single-stranded (pAAV EF1L synMMAB2) or self-complementary vectors (pAAV EF1a is syn MMAB5), were pseudoserotyped with an AAV9 or AAV8 capsid and tested for expression in an infection study using T1-4 and T1-8 HEK293 MMAB knock-out cell lines. Using the respective AAV8 or AAV9 viral vectors, the T1-4 and T1-8 MMAB 293 KO cells were infected over a range of doses, or moiety of infection (moi), between 5×10\hat{ }4 and 1×10\hat{ }6 (pAAV EF1L synMMAB2) or between approximately 2.5×10\hat{ }4 and 5×10\hat{ }5(pAAV EF1s synMMAB5). After 48 hours, the infected cells were harvested, extracted, and subjected to western blotting to detect MMAB expression from the AAV vector. T1-8 MMAB 293 KO cells infected with AAV8 vectors showed increasing MMAB expression as moi increased (FIGS. 6A-6B). T1-8 MMAB 293 KO cells infected with AAV9 vectors showed increasing MMAB expression as moi increased (FIGS. 7A-7B). T1-4 MMAB 293 KO cells infected with AAV8 vectors showed increasing MMAB expression as moi increased (FIGS. 8A-8B). T1-4 and T1-8 MMAB 293 KO cells infected with the AAV9 EF1L synMMAB2(ss) vector showed increasing MMAB expression as moi increased, with T1-8 MMAB 293 KO cells having higher expression than T1-4 MMAB 293 KO cells (FIGS. 9A-9B).

These results suggest that these plasmids could direct the expression of functional high titer AAV8 and AAV9 viral vectors in either genomic configuration, but further, that these vectors direct expression of properly processed MMAB.

### Example 6

This example demonstrates the efficacy of the AAV9 single-stranded and self-complementary synMMAB vectors at rescuing isolated methylmalonic acidemia (MMA) mouse models.

To test the efficacy of the single-strand and self-complementary AAV9 MMAB vectors, a series of in vivo gene therapy experiments using newly developed MMAB mouse models were performed. These MMAB mouse models are summarized in Table 6. Two severe mutations, the orthologue of a missense mutation recurrently noted in cb1B patients who received elective liver transplantation (p.Arg186Trp in MMAB in humans, MMAB^{R180W} in mice), and another that removed exons 3-7 (Mmab^{Δ3-7}), were engineered into the mouse germline. Because hepatic metabolism has been recognized as a critical to target for gene therapy in MMA, a germline rescue transgene, designed to restore expression of MMAB in the skeletal muscle under the control of the murine muscle creatine kinase (MCK) promoter, was knocked into the ROSA26 locus (ROSA26^{MCK-Mmab}), and subsequently crossed to the mice carrying Mmab mutations. Mmab^{Δ3-7/Δ3-7} and MMAB^{R180W/R180W} mice displayed lethality and massively increased plasma MMA (pMMA) levels (1150+/−177 uM, 360× WT), very much like patients with severe forms of cb1B MMA.

MMAB^{R180W/R180W};Tg^{ROSA26-MCK-Mmab} mice survived until weaning, but manifested pronounced MMAemia (520+/−79 uM, 200× WT), growth retardation, increased FGF21, and decreased ¹³C propionate oxidation.

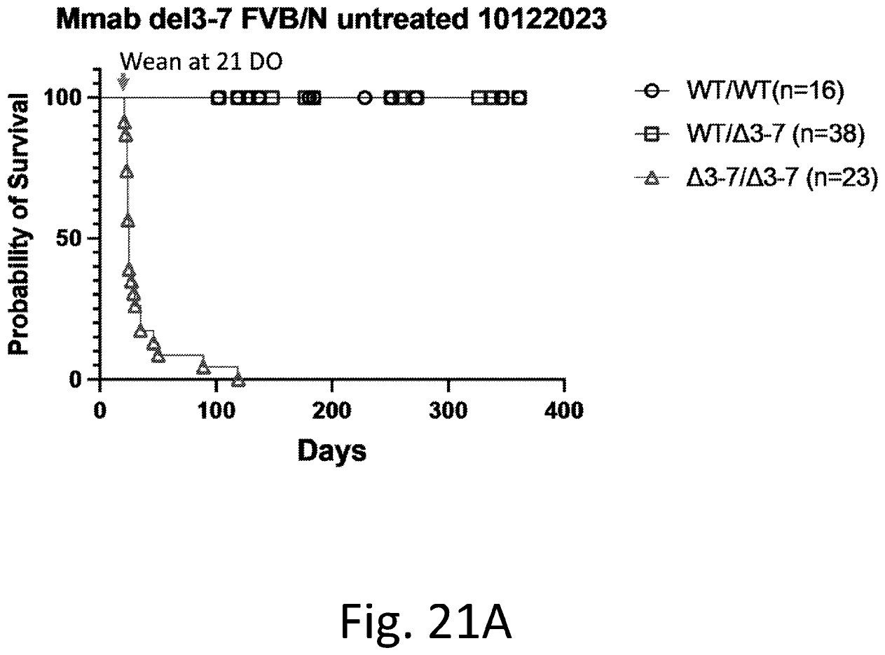

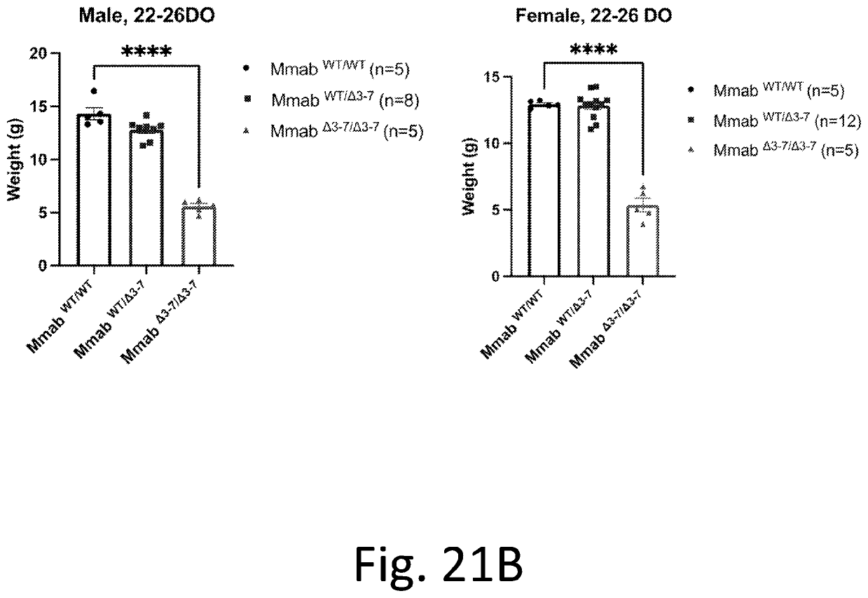

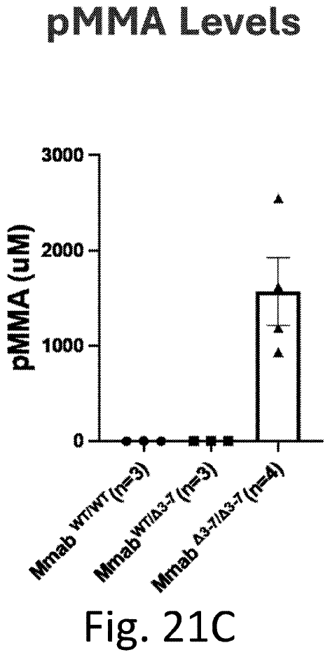

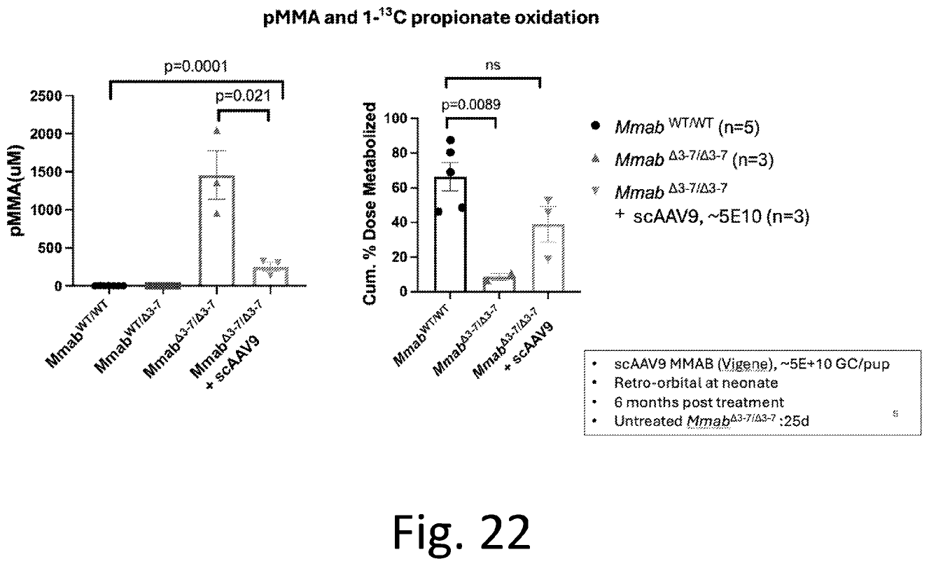

An additional mouse strain containing the Mmab^{Δ3-7} allele was generated using a different genetic background (Mmab^{Δ3-7/Δ3-7} (C57BL/6× FVB/N)). As depicted in FIG. 21A, Mmab^{Δ3-7/Δ3-7} (C57BL/6× FVB/N) mice survived until weaning but then survivability declines markedly. Also, as compared to wild-type mice, Mmab^{Δ3-7/Δ3-7} (C57BL/6× FVB/N) mice display severe runting (FIG. 21). Also, the pMMA levels for Mmab^{Δ3-7/Δ3-7} (C57BL/6× FVB/N) mice significantly exceed those of either heterozygotes or wild type mice (FIG. 21C). Mmab^{Δ3-7/Δ3-7} (C57BL/6× FVB/N) mice also manifest significantly decreased 1-¹³C propionate oxidation as compared to wild-type mice (see FIG. 22).

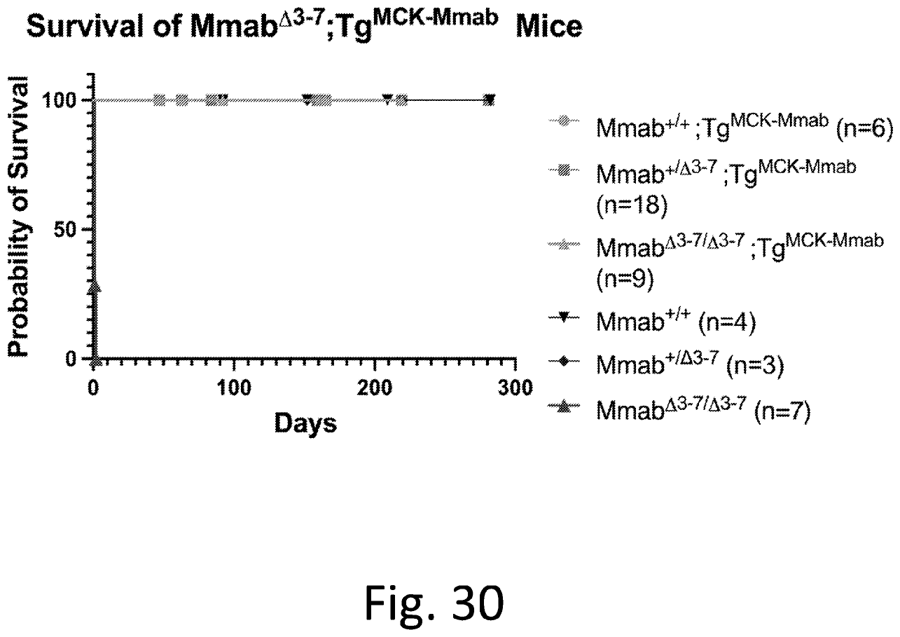

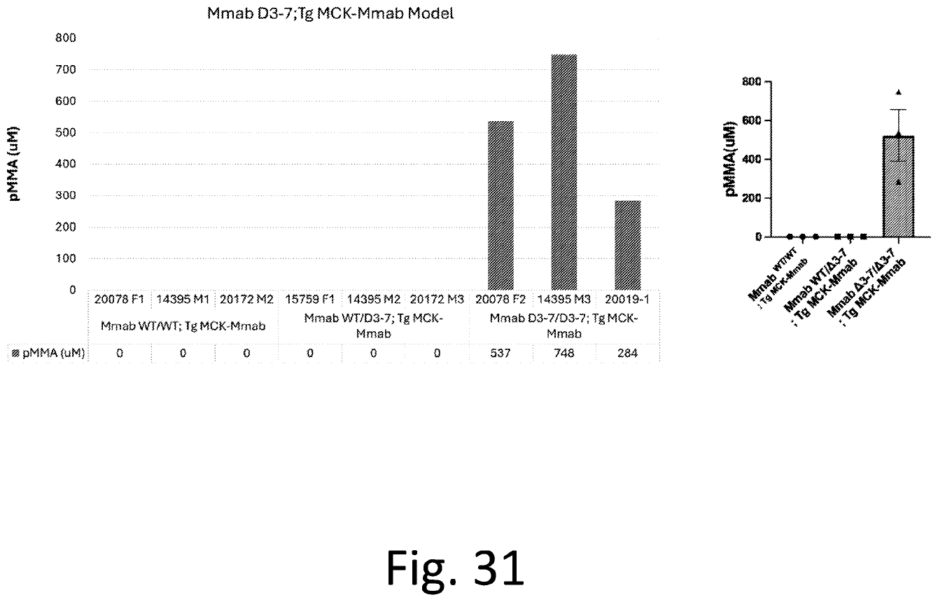

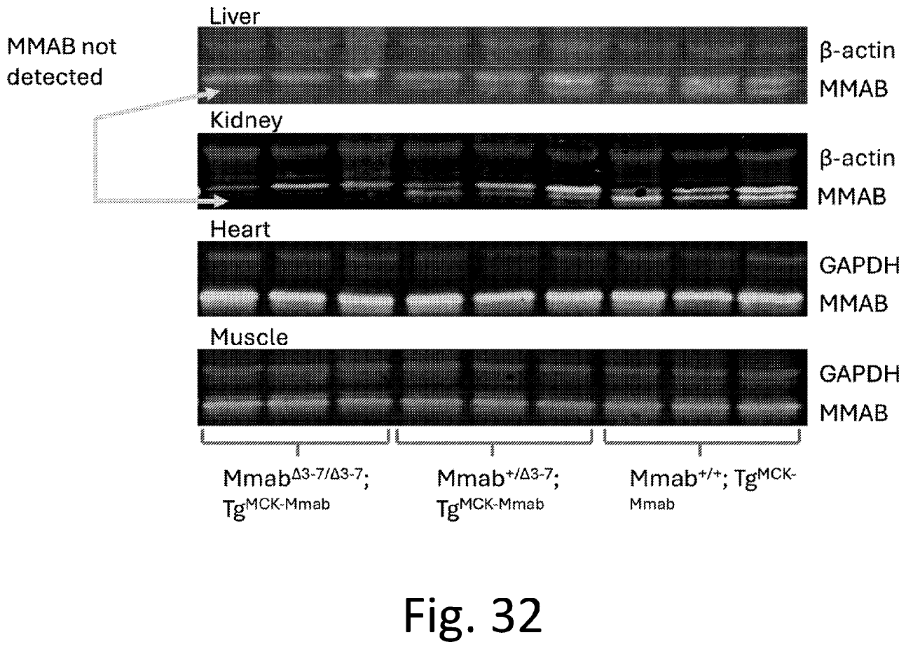

An additional hypomorphic mouse model strain containing the Mmab^{Δ3-7} allele and a transgenic MCK-Mmab construct (Tg^{MCK-Mmab}) was created in a C57BL6 murine background. The Tg^{MCK-Mmab} construct contains, from N- to C-termini, a polyA sequence, mouse Mmab CDs, and a mouse MCK promoter. This allele was introduced into a homozygotic Mmab^{Δ3-7} background to create the Mmab^{Δ3-7/Δ3-7};Tg^{MCK-Mmab} (C57BL/6) strain. This mouse model was assessed for survivability, the results of which are presented in FIG. 30. In comparison with homozygotic Mmab 3-7, which as discussed above is a lethal phenotype, all Mmab^{Δ3-7/Δ3-7};Tg^{MCK-Mmab} (C57BL/6) mice survived well past 200 days. This Mmab^{Δ3-7/Δ3-7};Tg^{MCK-Mmab} (C57BL/6) strain also was assessed for plasma MMA levels (see FIG. 31). In comparison with heterozygous Mmab^{WT/Δ3-7} mice and wild-type mice with respect to the Mmab trait (both also containing the Tg^{MCK-Mmab} allele), homozygotic Mmab^{Δ3-7} mice (i.e., Mmab^{Δ3-7/Δ3-7};Tg^{MCK-Mmab} (C57BL/6)) exhibited markedly high pMMA. Probing the MMAB expression in liver, kidney, heart, and muscle tissue (FIG. 32) revealed no expression of MMAB in Mmab^{Δ3-7/Δ3-7};Tg^{MCK-Mmab} (C57BL/6)) mice in liver and kidney, whereas MMAB expression was detected in heart and muscle (see FIG. 32).

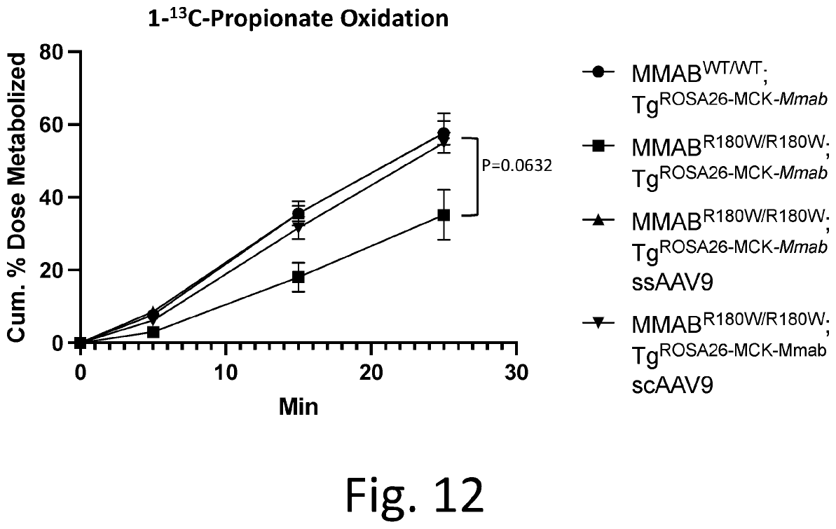

The ssAAV9 EF1L synMMAB2 or scAAV9 EF1s synMMAB5 vectors were assayed for in vivo efficacy in both severe model which display lethality around weaning (Mmab^{Δ3-7/Δ3-7}) and hypomorphic model (MMAB^{R180W/R180W}, Tg^{ROSA26-MCK-Mmab}) Neonatal treatment of Mmab^{Δ3-7/Δ3-7} mice (n=6) with the scAAV9 EF1a is synMMAB5, systemically delivered at a dose of approximately 5e10 GC/pup on DOL1, resulted in complete rescue from lethality, and full phenotypic correction persisting greater than 300 days (FIG. 12). Also, this cohort of Mmab^{Δ3-7/Δ3-7} mice displayed a significant reduction in pMMA as compared to untreated Mmab^{Δ3-7/Δ3-7} mice (FIG. 22, left panel) and an increase in 1-¹³C propionate oxidation to a level not statistically significantly different than wild-type mice (FIG. 22, right panel).

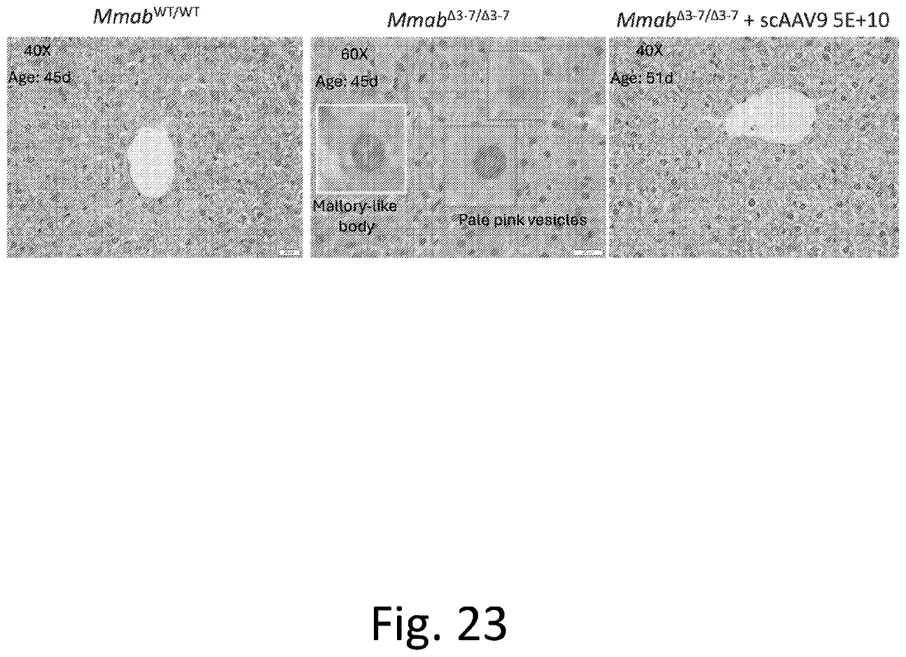

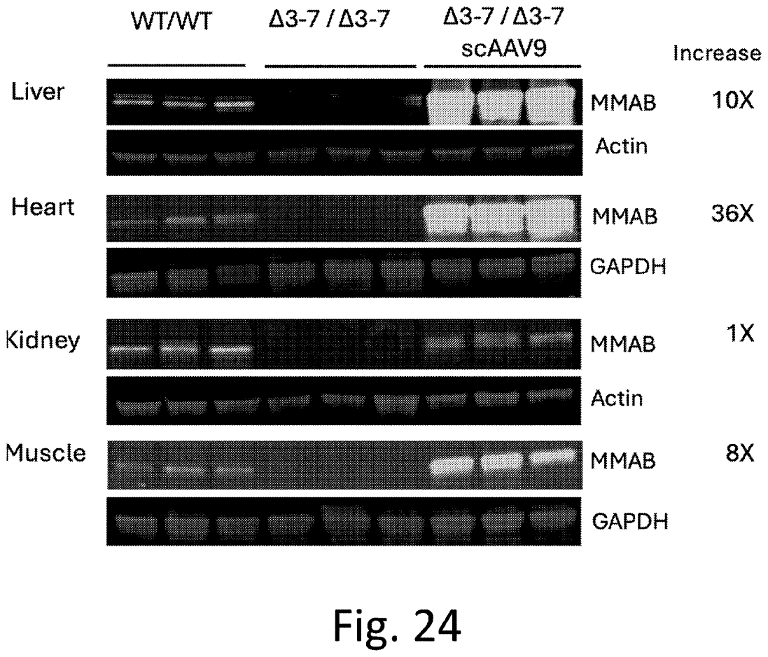

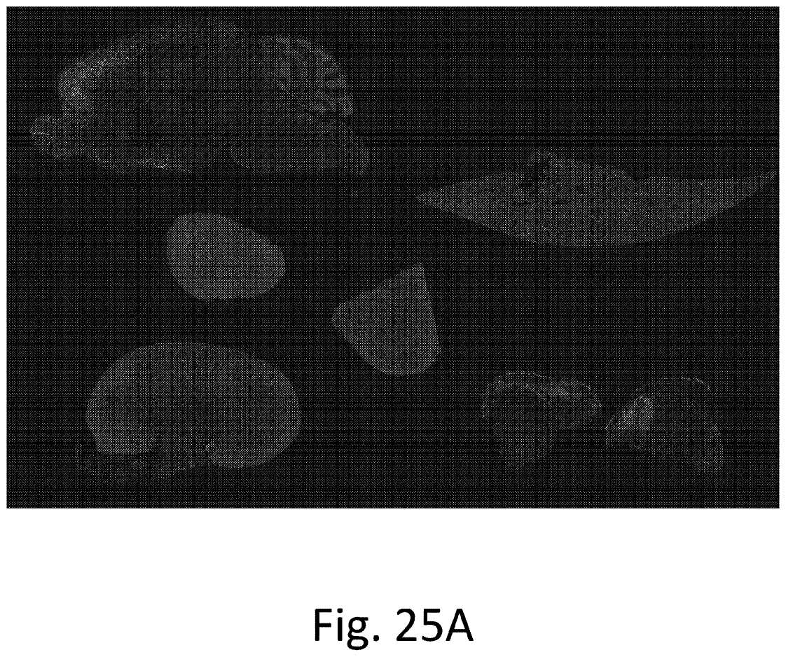

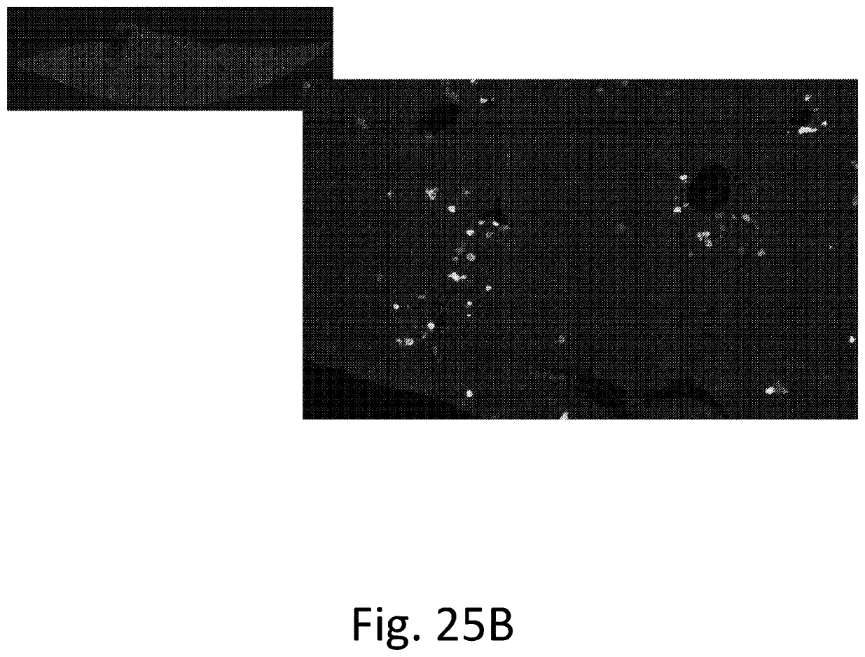

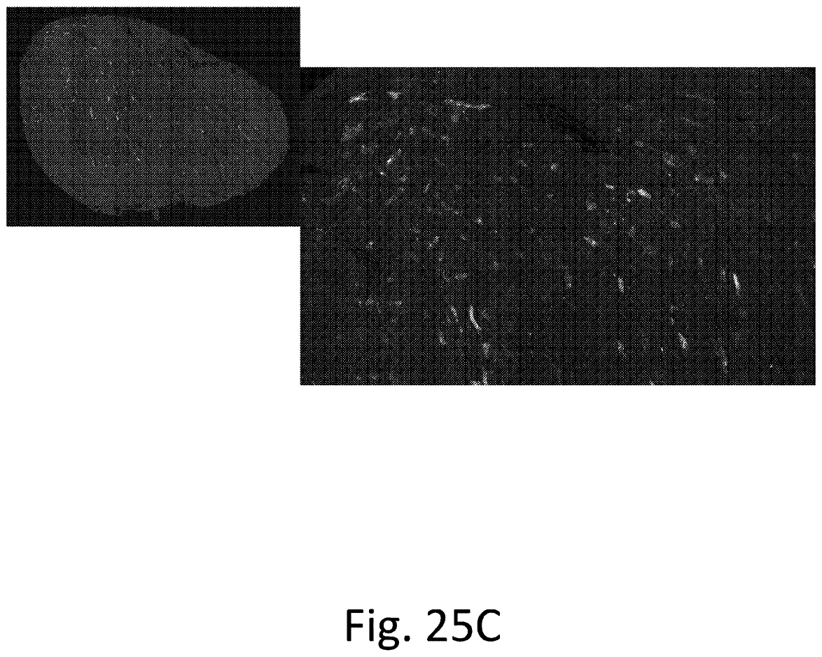

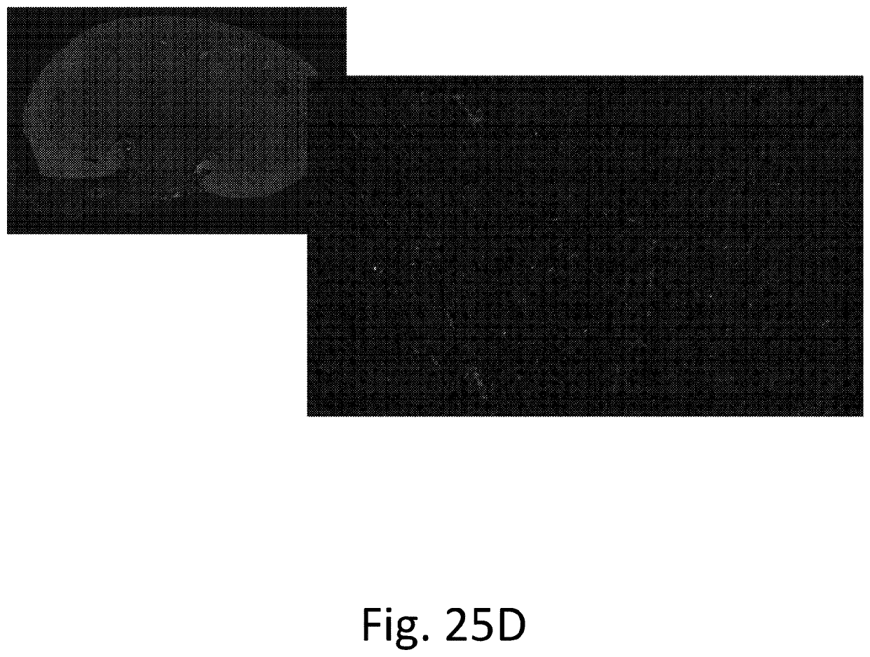

Mmab^{Δ3-7/Δ3-7} mice also were examined histologically to assess phenotype as compared to wild-type mice and to Mmab^{Δ3-7/Δ3-7} mice treated neonatally with scAAV9 EF1s synMMAB5 (see FIG. 23). While untreated Mmab^{Δ3-7/Δ3-7} mice exhibited Mallory-like bodies in liver tissue (FIG. 23, middle panel), that of Mmab^{Δ3-7/Δ3-7} mice treated with scAAV9 EF1a is synMMAB5 resembled liver tissue of wild-type mice (FIG. 23, left and right panels). Also, robust expression of MMAB was observed in liver, heart, kidney, and muscle tissue in Mmab^{Δ3-7/Δ3-7} mice six months following scAAV9 EF1s synMMAB5 (FIG. 24), including a 10-, 36-, and 8-fold increase of expression over wild-type mice for liver, heart, and kidney tissue, respectively. Additional histological data concerning expression of MMAB in tissue obtained from Mmab^{Δ3-7/Δ3-7}(C57BL/6× FVB/N) mice treated with scAAV9 EF1s synMMAB5 (5E10 GC/pup) are presented in FIGS. 25A-25D.

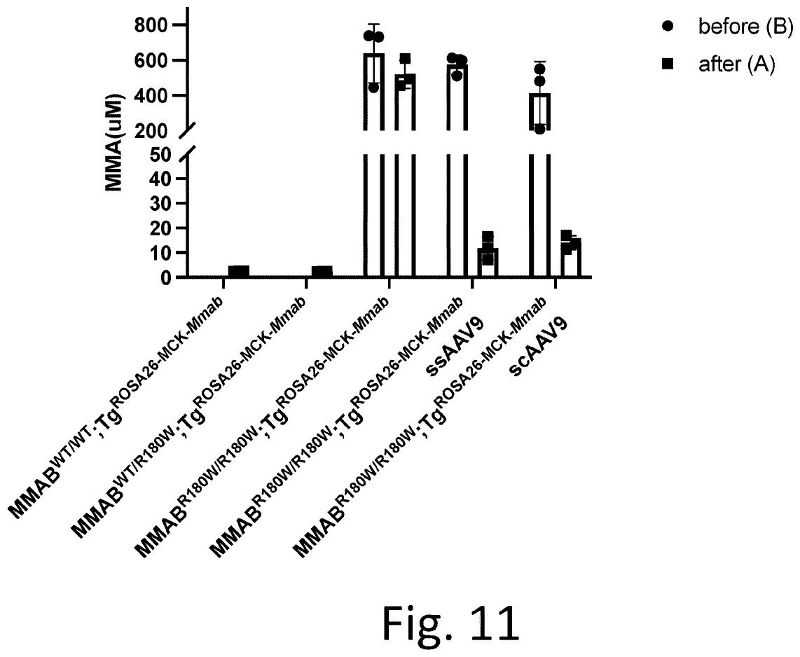

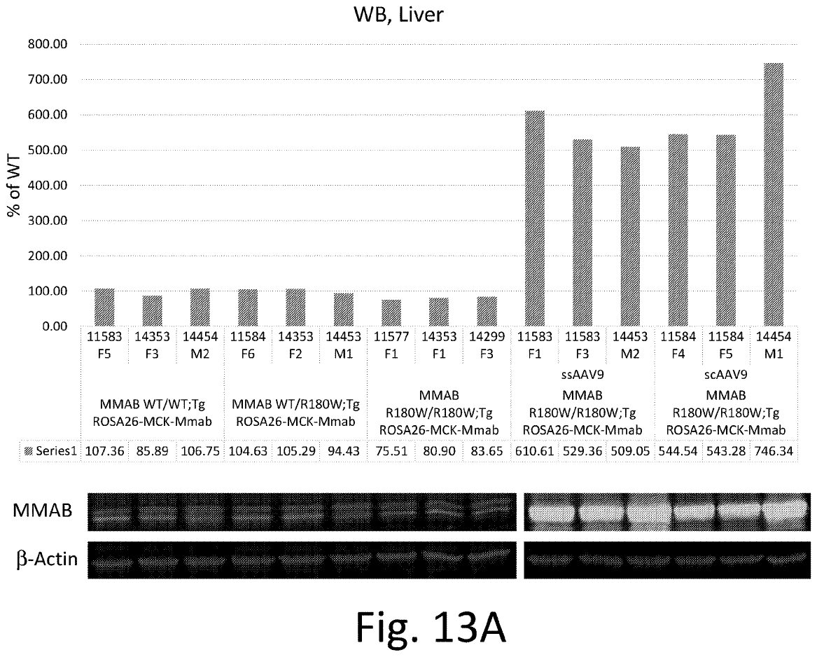

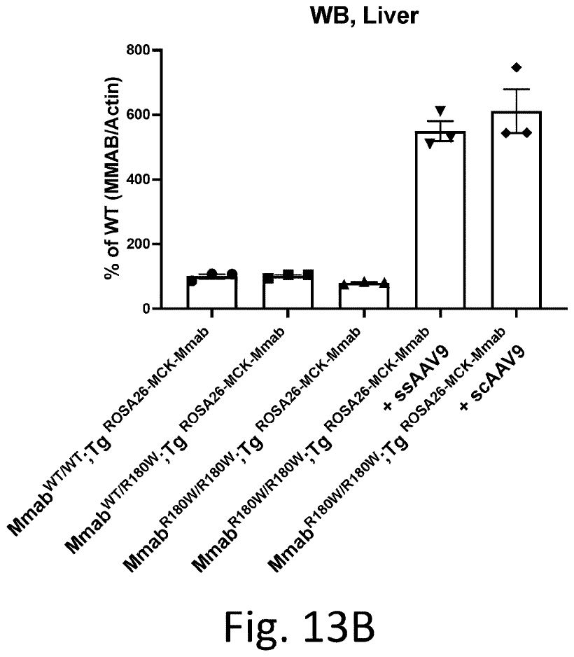

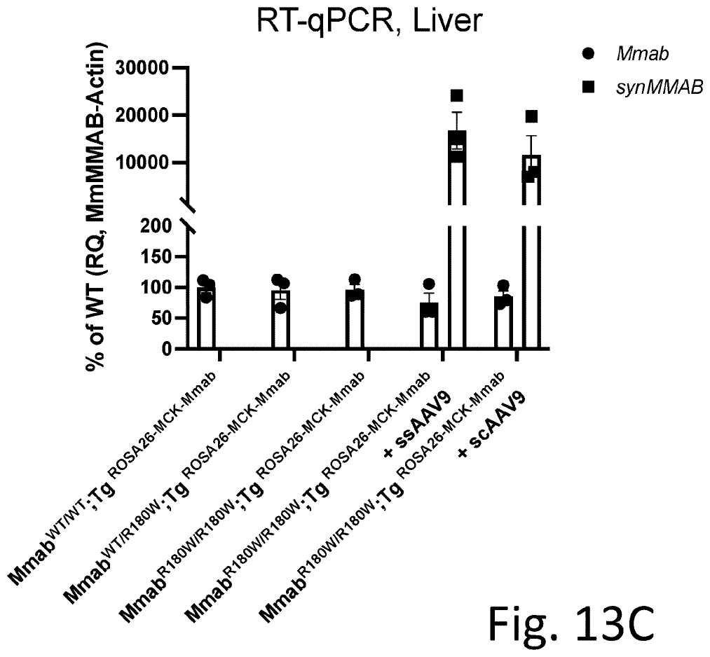

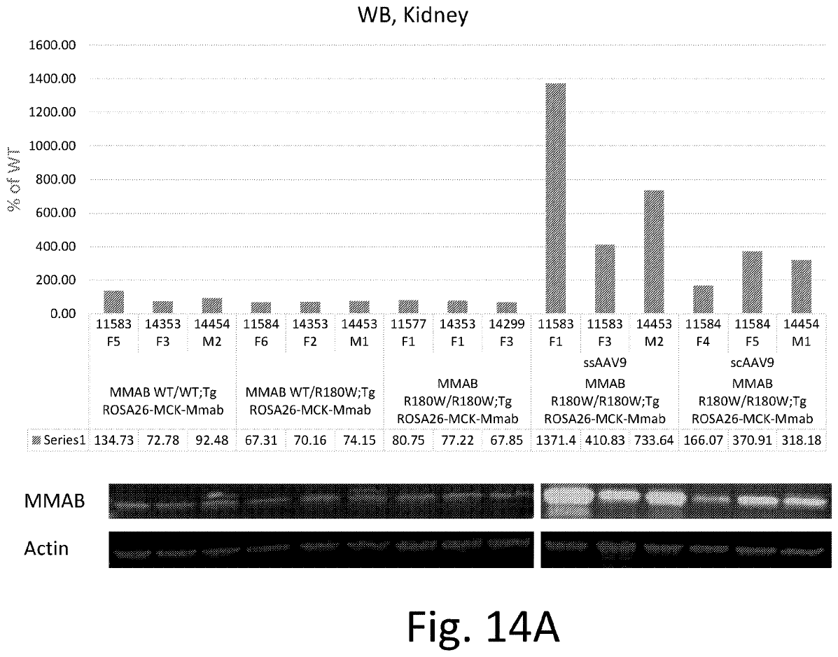

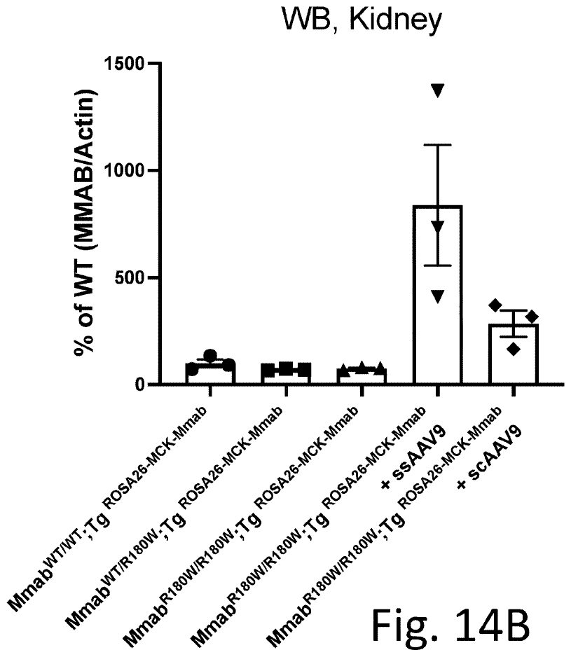

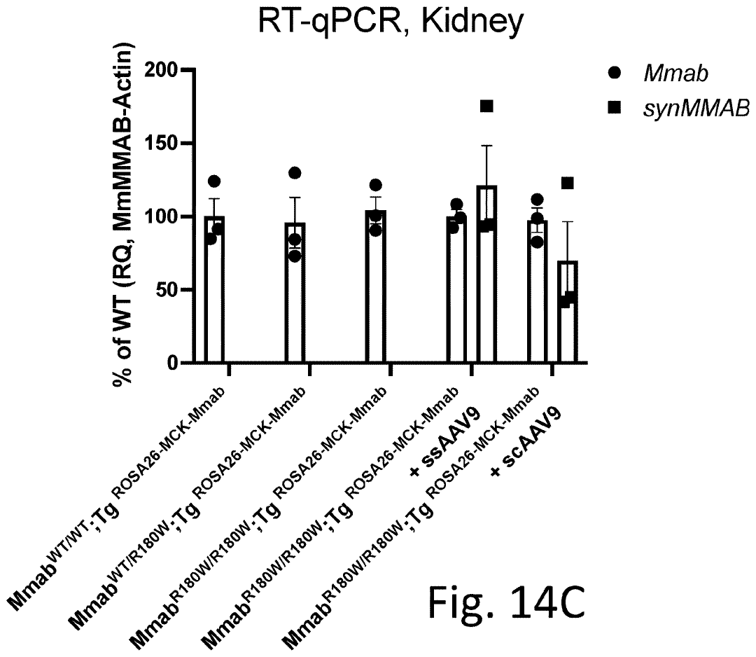

MMAB^{R180W/R180W} Tg^{ROSA26-MCK-Mmab} mice treated with either ssAAV9 EF1L synMMAB2 or scAAV9 EF1a is synMMAB5 vectors as young adults (n=6), given at a dose of 1e13 GC/kg, produced equally robust responses by 2 weeks, with a 38× reduction (12.9+/−3.7 uM) of the circulating plasma methylmalonic acid levels (FIG. 11 and Table 7) and restoration of 1-C¹³ propionate oxidation to wild type levels (FIG. 12). The ssAAV9 EF1L synMMAB2 or scAAV9 EF1s synMMAB5-treated MMAB^{R180W/R180W} Tg^{ROSA26-MCK-Mmab} mice also showed increased transgene expression in the liver as measured by both western blot analysis (FIGS. 13A and 13B) as well as RT-qPCR (FIG. 13C). The ssAAV9 EF1L synMMAB2 or scAAV9 EF1s synMMAB5 treated MMAB^{R180W/R180W} Tg^{ROSA26-MCK-Mmab} mice similarly showed increased transgene expression in the kidney as measured by both western blot analysis (FIGS. 14A and 14B) as well as RT-qPCR (FIG. 14C).

TABLE 6

| Allele | Phenotype | Comment | Uses |
|---|---|---|---|
| MMAB^{R180W} | Severe | Missense common in pts, | Rescue, dose finding, |
| Mmab^{Δ3-7} | Nonviable | CRM + | biomarkers |
| | | Deletion severe | |
| Mmab^{Q228X} | Milder | Common in pts, CRM+ | Dose finding |
| | Viable | | |
| ROSA26-MCK | WT | None | Transgene to rescue |
| Mmab | | | severe alleles from |
| Knock-In | | | germline |
| MMAB^{R180W} | Moderate/severe | Will resemble Mmut | Rescue, dose finding, |
| Mmab^{Δ3-7} | Viable | MCK mice (Manoli et al., | biomarkers |
| PLUS ROSA26- | | JCII 2018) | Hepatic effects of AAV |
| MCK-Mmab | | Stable into ROSA26 | |
| Mmab^{Q228X; Δ3-7} | Compound, | Common in pts, CRM+ | Dose finding |
| | milder | | Resistance to |
| | Viable | | Isoleucine/Valine |
| | | | challenge |

TABLE 7

| | | | After 2 |
|---|---|---|---|
| | | Before | weeks |
| | | pMMA | pMMA |
| Treatment | ID | (μM) | (μM) |
| MMAB^{WT/WT}; Tg ^{ROSA26-MCK-Mmab} | 11583 F5 | not | 2.59 |
| | | determined | |
| | | (ND) | |
| | 14353 F3 | ND | 2.53 |
| | 14454 M2 | ND | 2.68 |
| MMAB^{WT/R180W}; Tg ^{ROSA26-MCK-Mmab} | 11584 F6 | ND | 2.33 |
| | 14353 F2 | ND | 2.54 |
| | 14453 M1 | ND | 2.39 |
| MMAB^{R180W/R180W}, Tg^{ROSA26-MCK-Mmab} | 11577 F1 | 738.21 | 608.4 |
| untreated | 14353 F1 | 445.68 | 456.35 |
| | 14299 F3 | 731.44 | 494.95 |
| MMAB^{R180W/R180W}, Tg^{ROSA26-MCK-Mmab} | 11583 F1 | 511.47 | 7.08 |
| SSAAV9 | 11583 F3 | 612.4 | 11.85 |
| | 14453 M2 | 599.53 | 16.45 |
| MMAB^{R180W/R180W}, Tg^{ROSA26-MCK-Mmab} | 11584 F4 | 209.81 | 11.31 |
| scAAV9 | 11584 F5 | 481.28 | 16.99 |
| | 14454 M1 | 549.42 | 13.72 |

### Example 7

This example demonstrates the optimization of the AAV9 single-stranded and self-complementary synMMAB vectors for human translation.

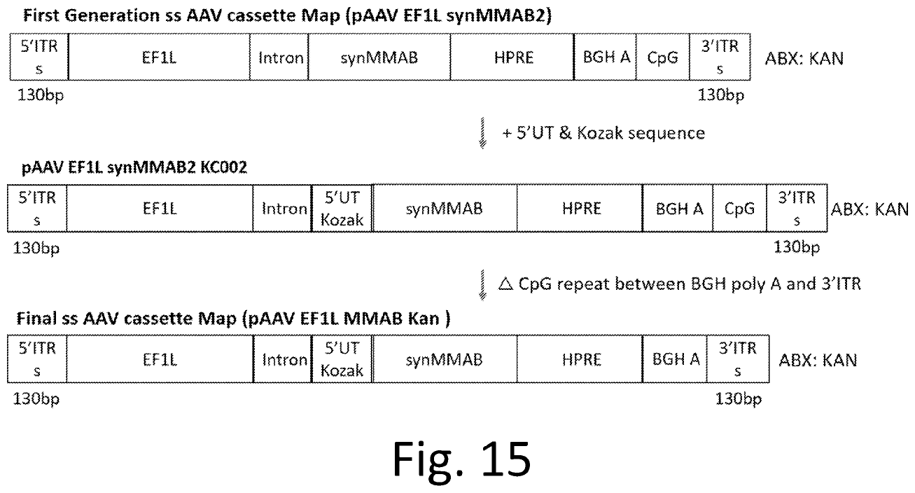

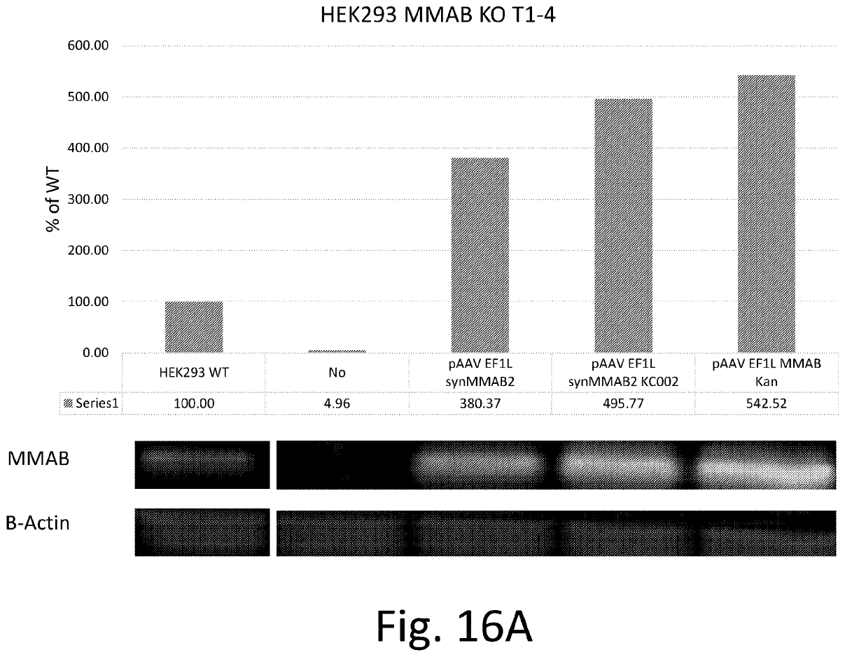

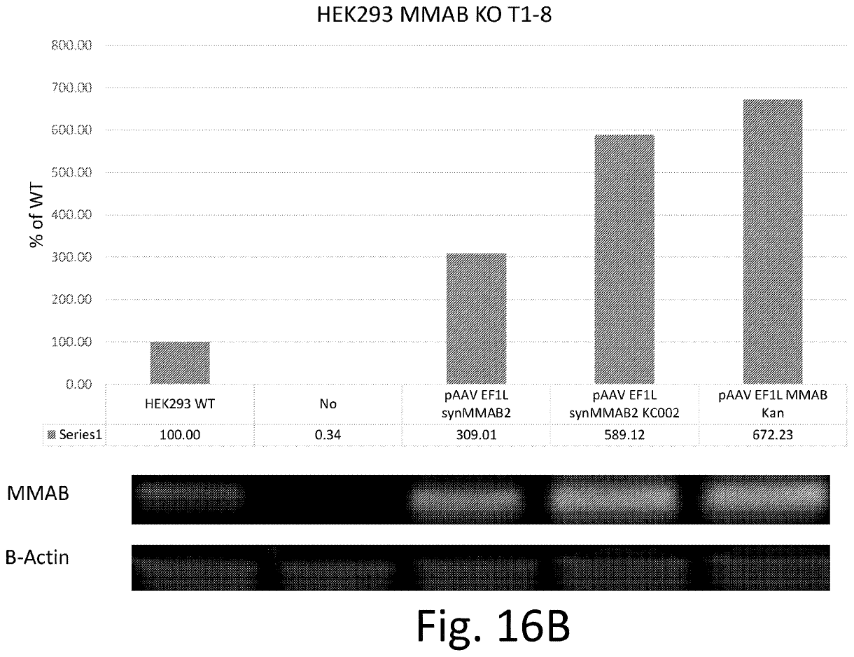

To further optimize the vectors for human translation, several design improvements were performed. For the ssAAV EF1L synMMAB2 vector, an optimized 5′ untranslated region was placed before the MA/AB start codon, and a CpG repeat between the polyA signal and 3′ITR was removed (FIG. 15). The resulting vector, pAAV EF1L MMAB Kan (ss), was transfected into the MA/AB KO cell lines and compared to the parent vector. The modified vector enabled even higher expression of MMAB in both the T1-4 MMAB KO cell line (FIG. 16A) and the T1-8 MMAB KO cell line (FIG. 16B). The sequence of this vector plasmid is presented in Table 8.

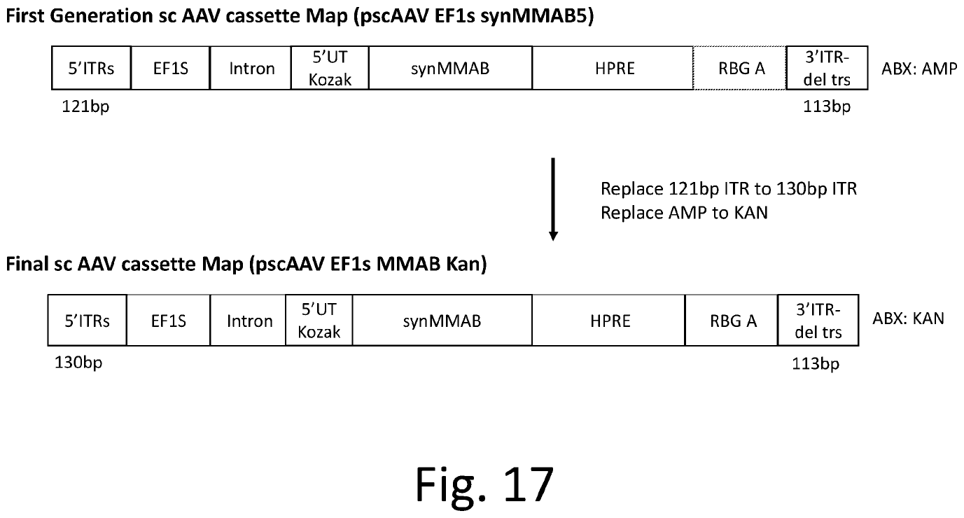

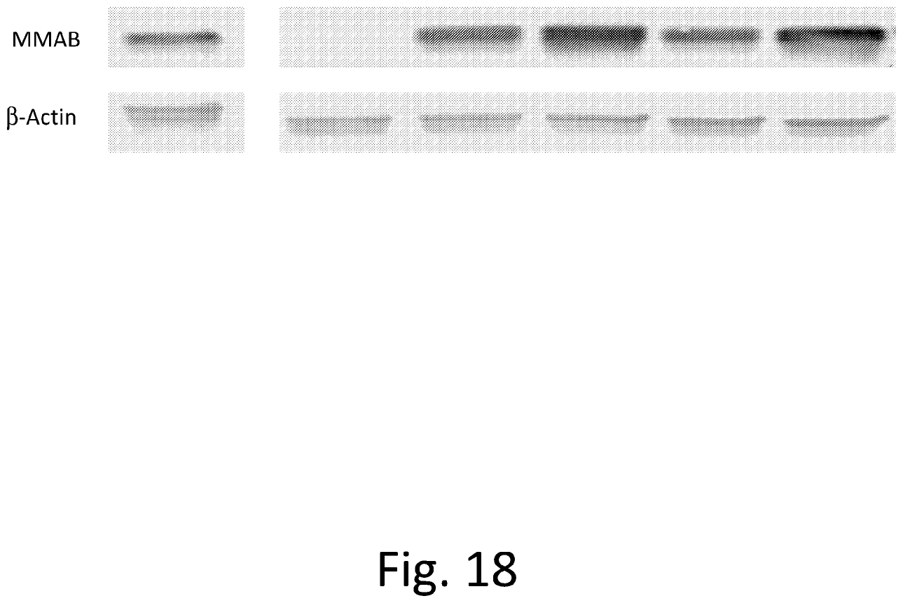

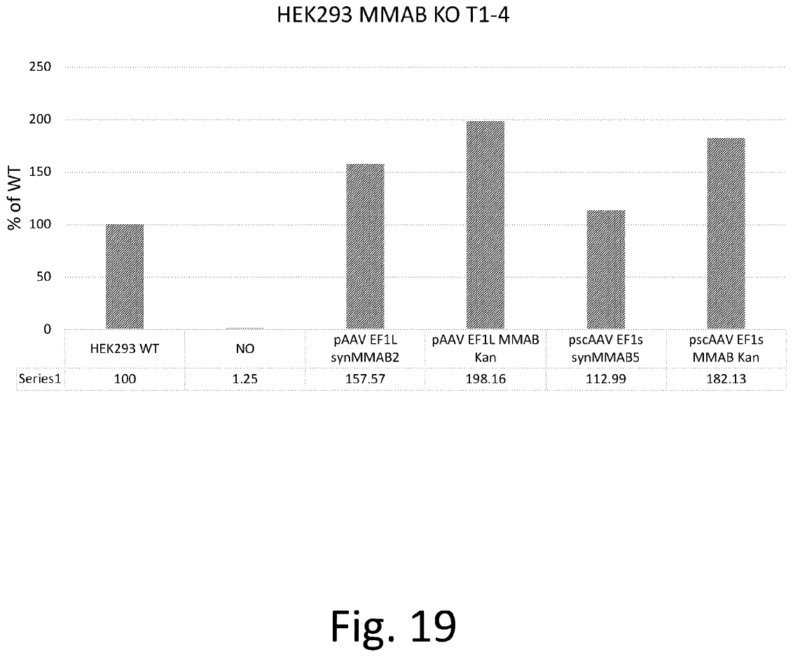

A different modification of the pscAAV EF1a is synMMAB5 vector was pursued. The 121 bp 5′ITR was swapped with a 130 bp version of a WT AAV2 ITR, and the antibiotic backbone was changed from ampicillin to kanamycin (FIG. 17). The sequence of this vector, pscAAV EF1a is MMAB Kan (sc), is presented in Table 9. The modified vector provided even higher expression of MMAB in the T1-4 MMAB KO cell line (FIG. 19).

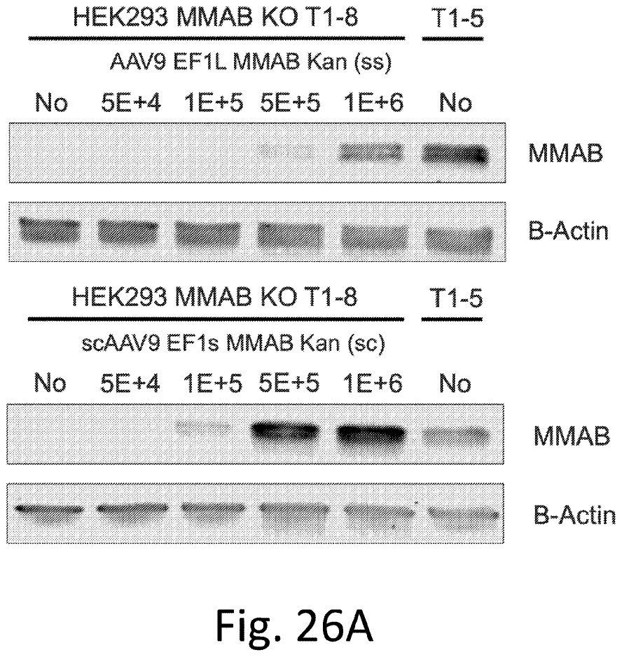

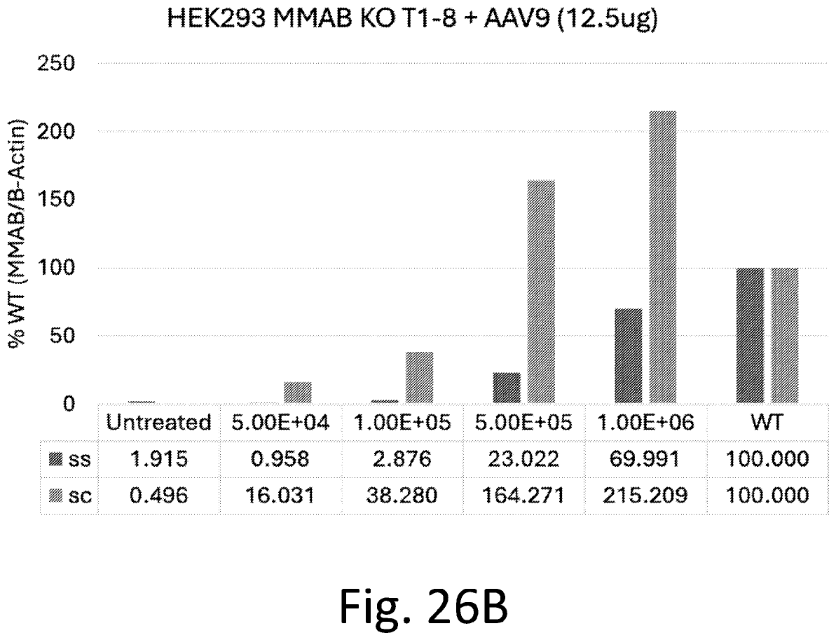

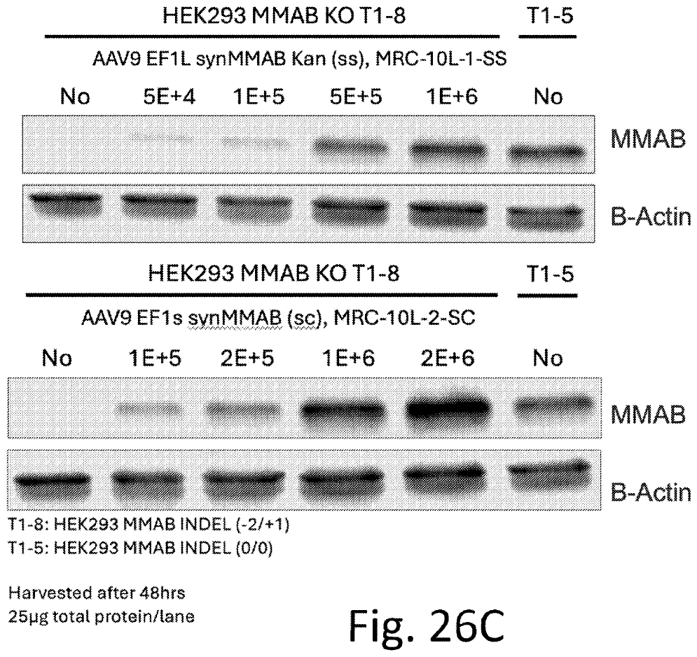

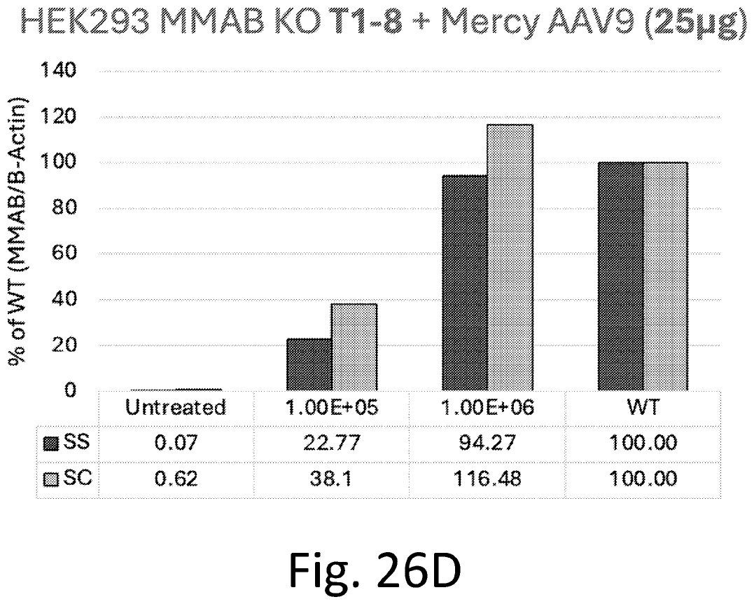

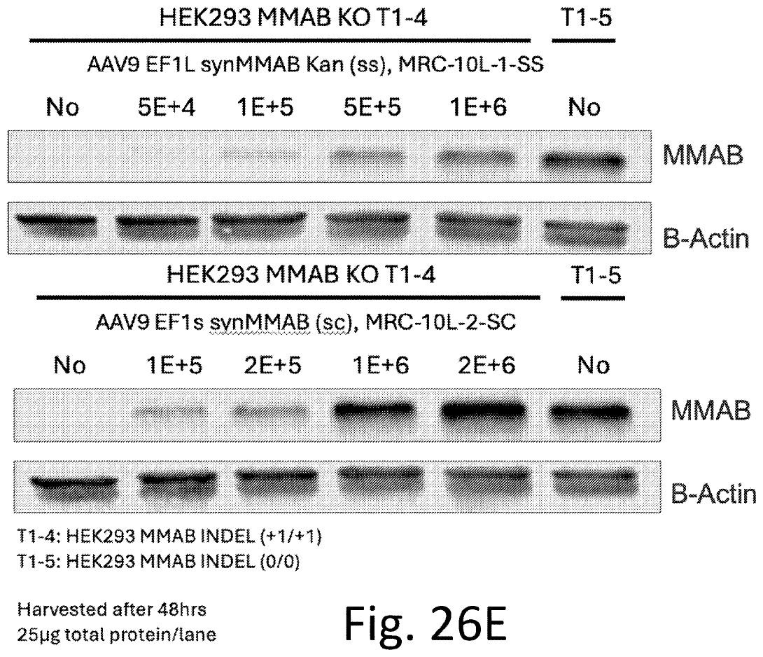

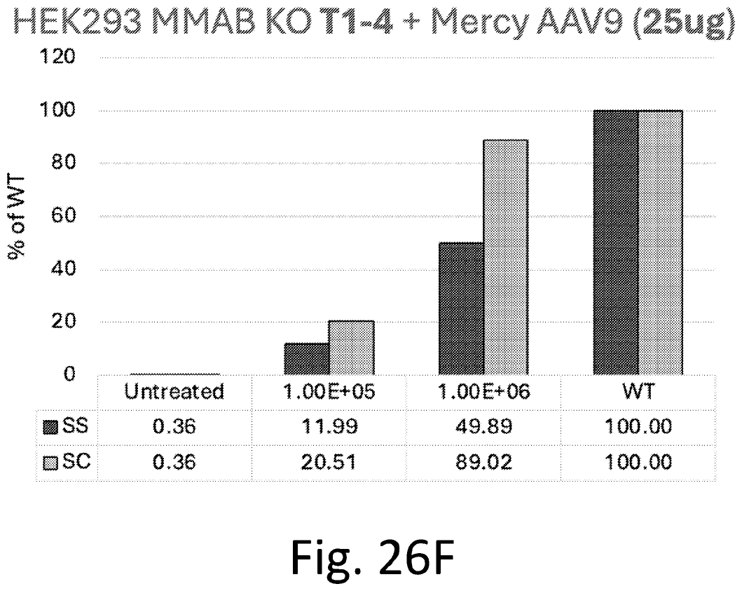

The optimized AAV9 EF1L MMAB Kan (ss) and AAV9 EF1s MMAB Kan (sc)) vectors were tested for their ability to correct the expression of MMAB in an infection study using T1-4 and T1-8 HEK293 A//AB knock-out cell lines, similarly, as described in Example 5 above. The cells were harvested after 48 hours, and for the Western blotting, total protein was loaded at 12.5 μg/lane (FIGS. 26A and 26B) or 25 μg/lane (FIGS. 26C. 26D, 26E or 26F). The results are presented in FIGS. 26A-26F. The data underlying FIGS. 26A and 26B were generated using a vector constructed by a different manufacturing laboratory than those underlying FIGS. 26C-26F, and the consistency of results further reveals the activity of these optimized vectors.

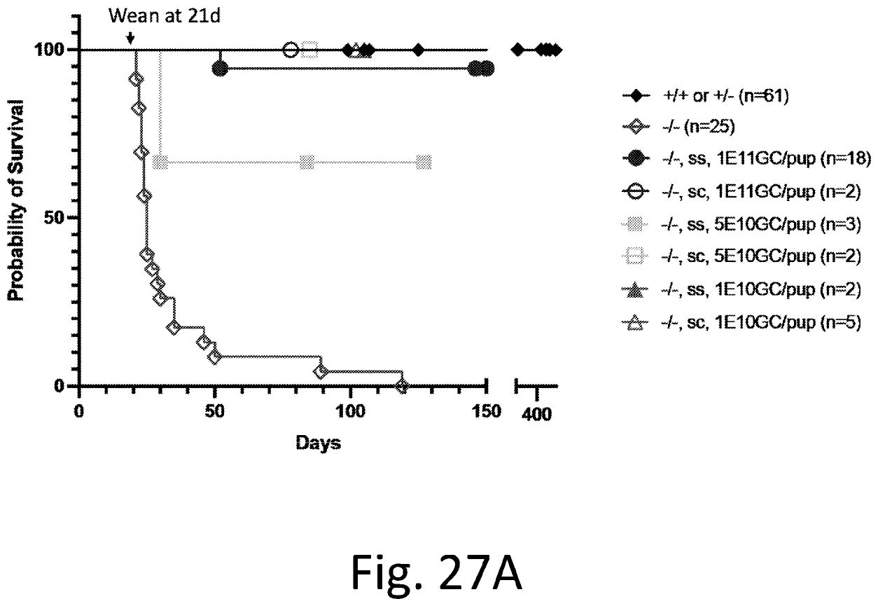

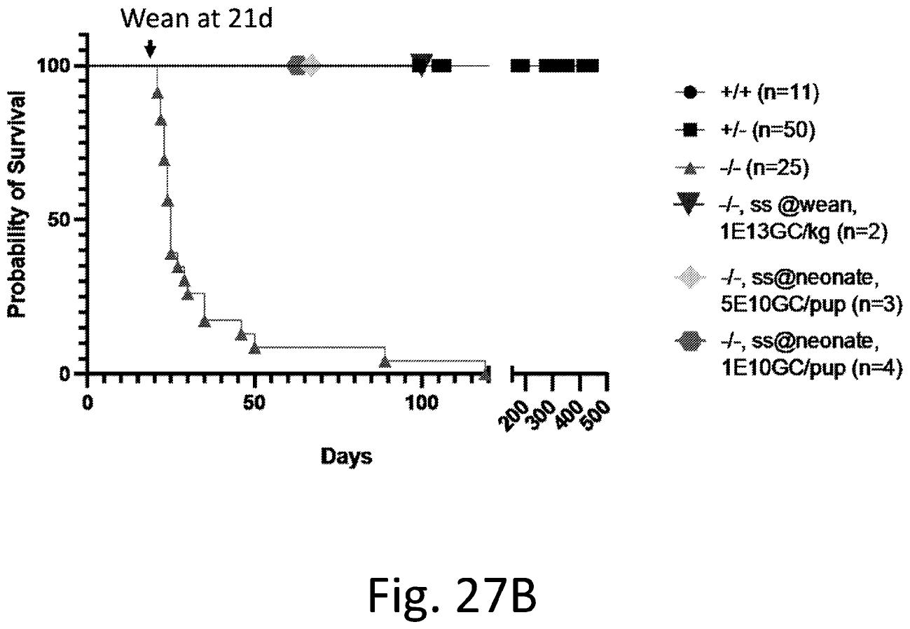

The optimized AAV9 EF1L MMAB Kan (ss) and AAV9 EF1s MMAB Kan (sc) vectors also were tested for their impact on the survival of Mmab^{Δ3-7/Δ3-7} (C57BL/6× FVB/N) mice. Neonatal treatment of Mmab^{Δ3-7/Δ3-7} mice treated with AAV9 EF1L MMAB Kan (ss) and AAV9 EF1s MMAB Kan (sc), systemically delivered at a dose of 1e10, 5e10, 1e11 on DOL1, resulted in rescue from lethality, and phenotypic correction persisting greater than 100 days (FIG. 27A). Treatment of Mmab^{Δ3-7/Δ3-7} mice with AAV9 EF1L MMAB Kan (ss), systemically delivered at a dose of 1E13 on DOL21(@wean), resulted in rescue from lethality, and phenotypic correction (FIG. 27B). The data in FIG. 27A reveal that the sc version of this vector permitted 100% survival rate during the assay period at all doses tested, as do the data in FIG. 27B for the ss version. The vectors employed in the experiment leading to FIG. 27A was constructed independently of that employed for FIGS. 27B, and the consistency of results further reveals the activity of these optimized vectors.

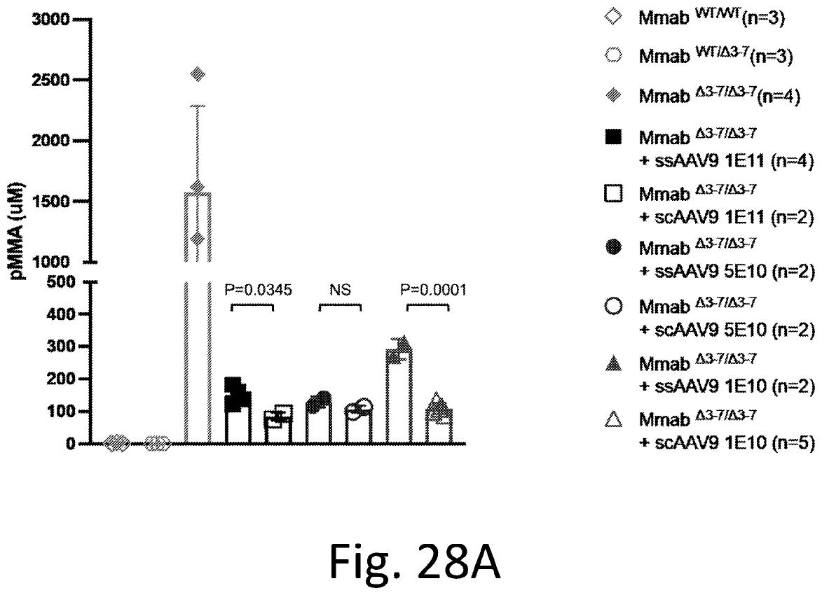

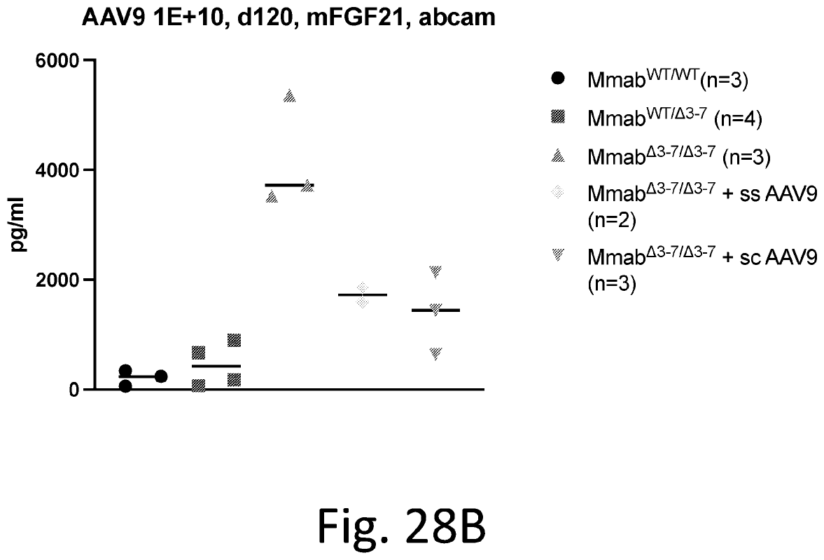

The optimized AAV9 EF1L MMAB Kan (ss) and AAV9 EF1s MMAB Kan (sc) vectors also were tested for their ability to rescue pMMA and for the levels of FGF produced in mice. Between 28 and 36 days following the neonatal DOL1 injection of either vector (dosed at 1e10, 5e10, or 1e11 GC/pup), a drastic reduction of pMMA was observed compared to untreated Mmab^{Δ3-7/Δ3-7} (C57BL/6× FVB/N) mice (FIG. 28A). Also, at DOL120, the FGF levels for the mice treated with the vectors (dosed at 1e10 GC/pup) was drastically reduced in comparison with untreated Mmab^{Δ3-7/Δ3-7} (C57BL/6× FVB/N) mice (FIG. 28B).

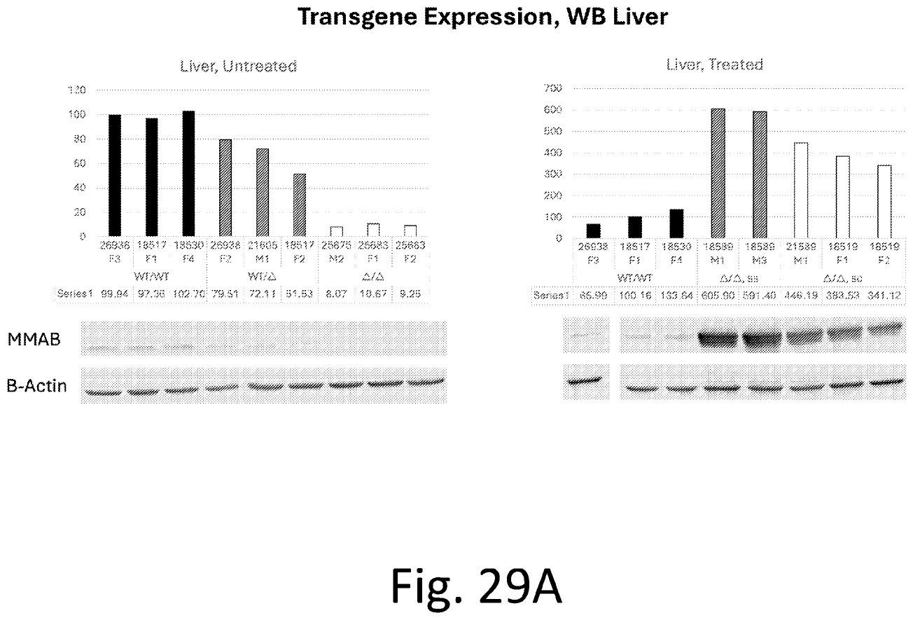

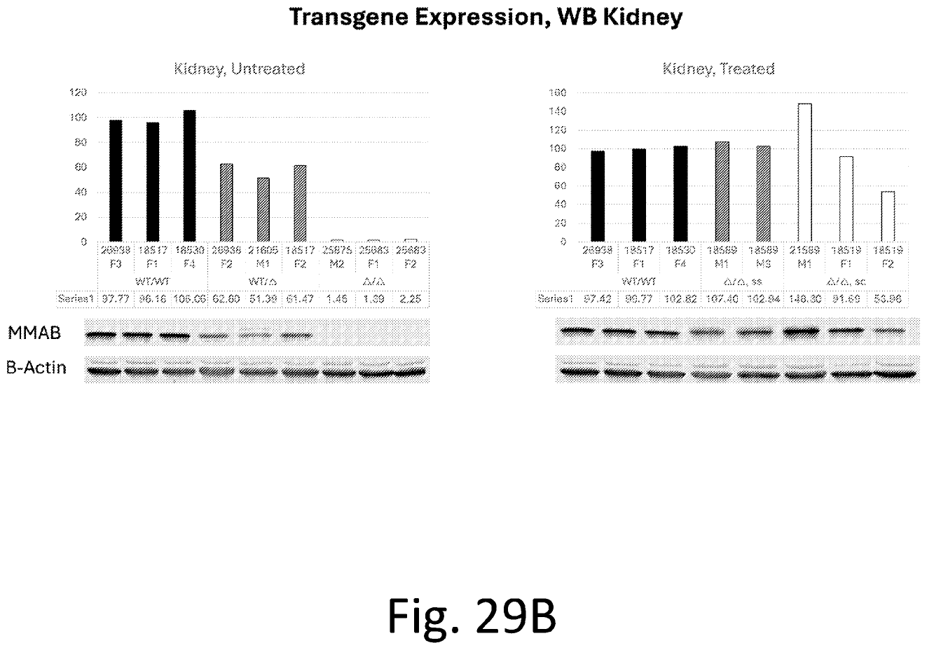

The MMAB transgene expression in liver and kidney following treatment with the AAV9 EF1L MMAB Kan (ss) and AAV9 EF1a is MMAB Kan (sc) vectors also was assayed. Treated or untreated wild-type, heterozygotic, or homozygotic Mmab^{Δ3-7/Δ3-7} (C57BL/6× FVB/N) mice were sacrificed at DOL120 and tissues processed and probed via Western blot analysis. The results (FIGS. 29A and 29B, right panels) reveal that treatment with the optimized V9 EF1L MMAB Kan (ss) and AAV9 EF1s MMAB Kan (sc)) vectors resulted in robust expression of MMAB in both tissues, dramatically exceeding the expression levels observed even for wild-type mice.

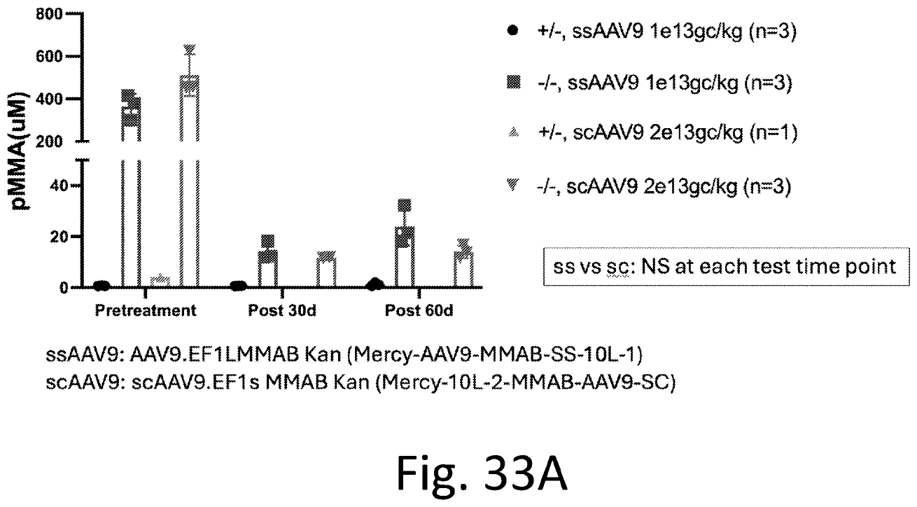

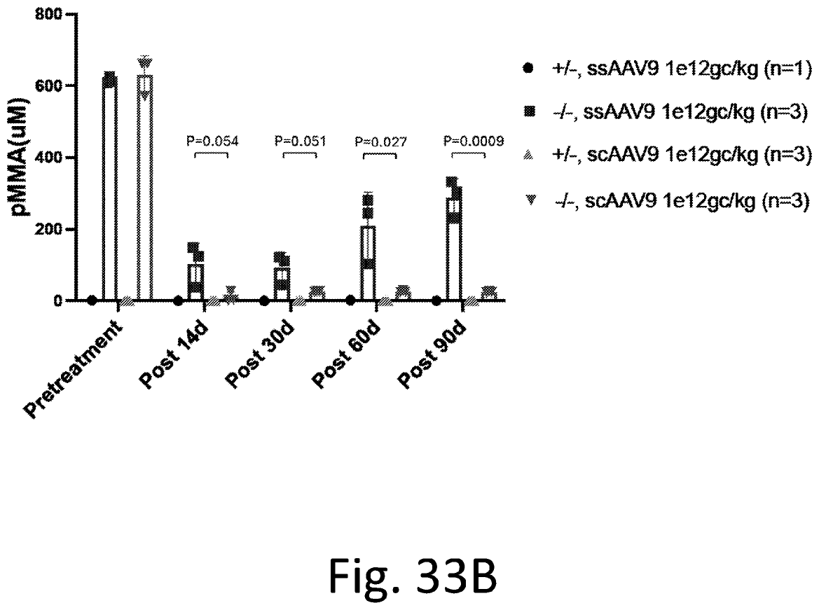

The AAV9 EF1L MMAB Kan (ss) and AAV9 EF1s MMAB Kan (sc)) vectors also were tested in the Mmab^{Δ3-7/Δ3-7};Tg^{MCK-Mmab} (C57BL/6) mouse model. As revealed by the data presented in FIG. 33, treatment with either of the two vectors markedly decreased plasma MMA levels when measured at 30 and 60 days from treatment, as compared with the neonatal cohort (pretreated) control.

In summary, the experiments described in these Examples have shown that a series of synthetic MMAB genes have been developed. It has been demonstrated that they have superior expression in a human MMAB knock out cell line. The new polynucleotides have been used to develop single-strand and self-complementary AAV8 and AAV9 vectors that can be used for neonatal and juvenile gene therapy for MMAB deficiency. The AAV9 vectors are potent in vivo, can produce virus well, and after systemic delivery at doses that approximate those given to humans in other AAV9 indications, mediate robust hepatorenal transgene expression in vivo. These studies provide a new class of vectors and AAV9 gene therapeutics to treat patients with MMAB deficiency, a severe form of MMA, and are suitable for immediate human translation.

TABLE 8
pAAV EFIL MMAB Kan Sequence-SEQ ID NO: 19

CTTTCCTGCGTTATCCCCTGATTCTGTGGATAACCGTATTACCGCCTTTGAGTGAG
CTGATACCGCTCGCCGCAGCCGAACGACCGAGCGCAGCGAGTCAGTGAGCGAGG
AAGCGGAAGAGCGCCCAATACGCAAACCGCCTCTCCCCGCGCGTTGGCCGATTC
ATTAATGCAGCTGGCACGACAGGTTTCCCGACTGGAAAGCGGGCAGTGAGCGCA
ACGCAATTAATACGCGTACCGCTAGCCAGGAAGAGTTTGTAGAAACGCAAAAAG
GCCATCCGTCAGGATGGCCTTCTGCTTAGTTTGATGCCTGGCAGTTTATGGCGGG
CGTCCTGCCCGCCACCCTCCGGGCCGTTGCTTCACAACGTTCAAATCCGCTCCCG
GCGGATTTGTCCTACTCAGGAGAGCGTTCACCGACAAACAACAGATAAAACGAA
AGGCCCAGTCTTCCGACTGAGCCTTTCGTTTTATTTGATGCCTGGCAGTTCCCTAC
TCTCGCGTTAACGCTAGCATGGATGTTTTCCCAGTCACGACGTTGTAAAACGACG
GCCAGTCTTAAGCTCGGGCCCCAAATAATGATTTTATTTTGACTGATAGTGACCT
GTTCGTTGCAACAAATTGATGAGCAATGCTTTTTTATAATGCCAACTTTGTACAA
AAAAGCAGGCTTCTAGACTGCGCGCTCGCTCGCTCACTGAGGCCGCCCGGGCAA
AGCCCGGGCGTCGGGCGACCTTTGGTCGCCCGGCCTCAGTGAGCGAGCGAGCGC
GCAGAGAGGGAGTGGCCAACTCCATCACTAGGGGTTCCTTTAATTAAGGCTCCG
GTGCCCGTCAGTGGGCAGAGCGCACATCGCCCACAGTCCCCGAGAAGTTGGGGG
GAGGGGTCGGCAATTGAACCGGTGCCTAGAGAAGGTGGCGCGGGGTAAACTGGG
AAAGTGATGTCGTGTACTGGCTCCGCCTTTTTCCCGAGGGTGGGGGAGAACCGTA
TATAAGTGCAGTAGTCGCCGTGAACGTTCTTTTTCGCAACGGGTTTGCCGCCAGA
ACACAGGTAAGTGCCGTGTGTGGTTCCCGCGGGCCTGGCCTCTTTACGGGTTATG
GCCCTTGCGTGCCTTGAATTACTTCCACCTGGCTGCAGTACGTGATTCTTGATCCC
GAGCTTCGGGTTGGAAGTGGGTGGGAGAGTTCGAGGCCTTGCGCTTAAGGAGCC
CCTTCGCCTCGTGCTTGAGTTGAGGCCTGGCCTGGGCGCTGGGGCCGCCGCGTGC
GAATCTGGTGGCACCTTCGCGCCTGTCTCGCTGCTTTCGATAAGTCTCTAGCCATT
TAAAATTTTTGATGACCTGCTGCGACGCTTTTTTTCTGGCAAGATAGTCTTGTAAA
TGCGGGCCAAGATCTGCACACTGGTATTTCGGTTTTTGGGGCCGCGGGCGGCGAC
GGGGCCCGTGCGTCCCAGCGCACATGTTCGGCGAGGCGGGGCCTGCGAGCGCGG
CCACCGAGAATCGGACGGGGGTAGTCTCAAGCTGGCCGGCCTGCTCTGGTGCCT
GGCCTCGCGCCGCCGTGTATCGCCCCGCCCTGGGCGGCAAGGCTGGCCCGGTCG
GCACCAGTTGCGTGAGCGGAAAGATGGCCGCTTCCCGGCCCTGCTGCAGGGAGC
TCAAAATGGAGGACGCGGCGCTCGGGAGAGCGGGCGGGTGAGTCACCCACACA
AAGGAAAAGGGCCTTTCCGTCCTCAGCCGTCGCTTCATGTGACTCCACGGAGTAC
CGGGCGCCGTCCAGGCACCTCGATTAGTTCTCGAGCTTTTGGAGTACGTCGTCTT
TAGGTTGGGGGGAGGGGTTTTATGCGATGGAGTTTCCCCACACTGAGTGGGTGG
AGACTGAAGTTAGGCCAGCTTGGCACTTGATGTAATTCTCCTTGGAATTTGCCCT
TTTTGAGTTTGGATCTTGGTTCATTCTCAAGCCTCAGACAGTGGTTCAAAGTTTTT
TTCTTCCATTTCAGGTGTCGTGAGCTAGAGCTTTATTGCGGTAGTTTATCACAGTT
AAATTGCTAACGCAGTCAGTGCTTCTGACACAACAGTCTCGAACTTAAGCTGCAG
AAGTTGGTCGTGAGGCACTGGGCAGGTAAGTATCAAGGTTACAAGACAGGTTTA
AGGAGACCAATAGAAACTGGGCTTGTCGAGACAGAGAAGACTCTTGCGTTTCTG
ATAGGCACCTATTGGTCTTACTGACATCCACTTTGCCTTTCTCTCCACAGGTGTCC
ACTCCCAGTTCAATTACAGCTCTTAAGGCTAGAGTACTGAATTCAAATAAGAGAG
AAAAGAAGAGTAAGAAGAAATATAAGAGCCACCATGGCGGTCTGCGGATTGGGT
TCACGATTGGGCCTTGGGTCTAGGCTCGGCCTGCGAGGTTGTTTCGGAGCTGCTA
GATTGTTGTACCCACGATTCCAAAGCAGGGGACCACAAGGTGTGGAAGACGGTG
ACAGGCCTCAGCCCTCAAGCAAAACCCCTCGGATTCCGAAAATATATACAAAGA
CAGGAGACAAAGGCTTTTCAAGCACGTTTACTGGCGAACGAAGGCCTAAAGACG
ATCAAGTTTTTGAGGCGGTAGGGACAACTGACGAACTTTCATCTGCGATAGGCTT
CGCGCTCGAGTTGGTGACGGAGAAGGGTCATACATTCGCTGAAGAACTTCAGAA
AATTCAGTGTACGCTCCAAGATGTTGGCAGTGCGCTTGCGACACCTTGCAGCTCA
GCAAGGGAAGCGCATTTGAAGTACACCACTTTCAAAGCGGGCCCGATTCTCGAA
CTTGAACAATGGATTGACAAATATACAAGCCAACTGCCGCCTCTTACCGCCTTCA
TCCTCCCTAGTGGTGGCAAAATTTCTTCCGCGCTTCATTTTTGTAGGGCCGTGTGT
CGCCGCGCCGAGAGACGGGTGGTTCCGTTGGTACAGATGGGTGAAACAGATGCA
AACGTGGCAAAGTTCCTTAACCGCCTCAGCGACTACTTGTTCACTCTCGCTAGGT
ATGCCGCCATGAAAGAAGGGAATCAAGAGAAGATCTATATGAAGAATGACCCGA
GCGCTGAAAGCGAGGGACTTTGAGGCCTCGAGATAACAGGCCTATTGATTGGAA
AGTTTGTCAACGAATTGTGGGTCTTTTGGGGTTTGCTGCCCCTTTTACGCAATGTG
GATATCCTGCTTTAATGCCTTTATATGCATGTATACAAGCAAAACAGGCTTTTACT
TTCTCGCCAACTTACAAGGCCTTTCTCAGTAAACAGTATATGACCCTTTACCCCGT
TGCTCGGCAACGGCCTGGTCTGTGCCAAGTGTTTGCTGACGCAACCCCCACTGGT
TGGGGCTTGGCCATAGGCCATCAGCGCATGCGTGGAACCTTTGTGTCTCCTCTGC
CGATCCATACTGCGGAACTCCTAGCCGCTTGTTTTGCTCGCAGCAGGTCTGGAGC
AAACCTCATCGGGACCGACAATTCTGTCGTACTCTCCCGCAAGTATACATCGTTT
CCATGGCTGCTAGGCTGTGCTGCCAACTGGATCCTGCGCGGGACGTCCTTTGTTT
ACGTCCCGTCGGCGCTGAATCCCGCGGACGACCCCTCCCGGGGCCGCTTGGGGCT
CTACCGCCCGCTTCTCCGTCTGCCGTACCGTCCGACCACGGGGCGCACCTCTCTTT
ACGCGGACTCCCCGTCTGTGCCTTCTCATCTGCCGGACCGTGTGCACTTCGCTTCA
CCTCTGCACGTCGCATGGAGGCCACCGTGAACGCCCACCGGAACCTGCCCAAGG
TCTTGCATAAGAGGACTCTTGGACTTTCAGCAATGTCATCGATATCGTCGACTCG
CTGATCAGCCTCGACTGTGCCTTCTAGTTGCCAGCCATCTGTTGTTTGCCCCTCCC
CCGTGCCTTCCTTGACCCTGGAAGGTGCCACTCCCACTGTCCTTTCCTAATAAAAT
GAGGAAATTGCATCGCATTGTCTGAGTAGGTGTCATTCTATTCTGGGGGGTGGGG
TGGGGCAGGACAGCAAGGGGGAGGATTGGGAAGACAATAGCAGGCATGCTGGG
GATGCGGTGGGCTCTATGGCTTCTGAGGCGGAAAGAACCAGCTGGGGCTCGACT
AGACTAGTCCTGCAGGTACCTAGGAACCCCTAGTGATGGAGTTGGCCACTCCCTC
TCTGCGCGCTCGCTCGCTCACTGAGGCCGGGCGACCAAAGGTCGCCCGACGCCC
GGGCTTTGCCCGGGCGGCCTCAGTGAGCGAGCGAGCGCGCAGCATATGACCCAG
CTTTCTTGTACAAAGTTGGCATTATAAGAAAGCATTGCTTATCAATTTGTTGCAAC
GAACAGGTCACTATCAGTCAAAATAAAATCATTATTTGCCATCCAGCTGATATCC
CCTATAGTGAGTCGTATTACATGGTCATAGCTGTTTCCTGGCAGCTCTGGCCCGT
GTCTCAAAATCTCTGATGTTACATTGCACAAGATAAAAATATATCATCATGAACA
ATAAAACTGTCTGCTTACATAAACAGTAATACAAGGGGTGTTATGAGCCATATTC
AACGGGAAACGTCGAGGCCGCGATTAAATTCCAACATGGATGCTGATTTATATG
GGTATAAATGGGCTCGCGATAATGTCGGGCAATCAGGTGCGACAATCTATCGCTT
GTATGGGAAGCCCGATGCGCCAGAGTTGTTTCTGAAACATGGCAAAGGTAGCGT
TGCCAATGATGTTACAGATGAGATGGTCAGACTAAACTGGCTGACGGAATTTATG
CCTCTTCCGACCATCAAGCATTTTATCCGTACTCCTGATGATGCATGGTTACTCAC
CACTGCGATCCCCGGAAAAACAGCATTCCAGGTATTAGAAGAATATCCTGATTCA
GGTGAAAATATTGTTGATGCGCTGGCAGTGTTCCTGCGCCGGTTGCATTCGATTC
CTGTTTGTAATTGTCCTTTTAACAGCGATCGCGTATTTCGTCTCGCTCAGGCGCAA
TCACGAATGAATAACGGTTTGGTTGATGCGAGTGATTTTGATGACGAGCGTAATG
GCTGGCCTGTTGAACAAGTCTGGAAAGAAATGCATAAACTTTTGCCATTCTCACC
GGATTCAGTCGTCACTCATGGTGATTTCTCACTTGATAACCTTATTTTTGACGAGG
GGAAATTAATAGGTTGTATTGATGTTGGACGAGTCGGAATCGCAGACCGATACC
AGGATCTTGCCATCCTATGGAACTGCCTCGGTGAGTTTTCTCCTTCATTACAGAA
ACGGCTTTTTCAAAAATATGGTATTGATAATCCTGATATGAATAAATTGCAGTTT
CATTTGATGCTCGATGAGTTTTTCTAATCAGAATTGGTTAATTGGTTGTAACACTG
GCAGAGCATTACGCTGACTTGACGGGACGGCGCAAGCTCATGACCAAAATCCCT
TAACGTGAGTTACGCGTCGTTCCACTGAGCGTCAGACCCCGTAGAAAAGATCAA
AGGATCTTCTTGAGATCCTTTTTTTCTGCGCGTAATCTGCTGCTTGCAAACAAAAA
AACCACCGCTACCAGCGGTGGTTTGTTTGCCGGATCAAGAGCTACCAACTCTTTT
TCCGAAGGTAACTGGCTTCAGCAGAGCGCAGATACCAAATACTGTTCTTCTAGTG
TAGCCGTAGTTAGGCCACCACTTCAAGAACTCTGTAGCACCGCCTACATACCTCG
CTCTGCTAATCCTGTTACCAGTGGCTGCTGCCAGTGGCGATAAGTCGTGTCTTAC
CGGGTTGGACTCAAGACGATAGTTACCGGATAAGGCGCAGCGGTCGGGCTGAAC
GGGGGGTTCGTGCACACAGCCCAGCTTGGAGCGAACGACCTACACCGAACTGAG
ATACCTACAGCGTGAGCTATGAGAAAGCGCCACGCTTCCCGAAGGGAGAAAGGC
GGACAGGTATCCGGTAAGCGGCAGGGTCGGAACAGGAGAGCGCACGAGGGAGC
TTCCAGGGGGAAACGCCTGGTATCTTTATAGTCCTGTCGGGTTTCGCCACCTCTG
ACTTGAGCGTCGATTTTTGTGATGCTCGTCAGGGGGGCGGAGCCTATGGAAAAAC
GCCAGCAACGCGGCCTTTTTACGGTTCCTGGCCTTTTGCTGGCCTTTTGCTCACAT
GTT

TABLE 9
pscAAV EF1s MMAB Kan Sequence-SEQ ID NO: 20

CTTTCCTGCGTTATCCCCTGATTCTGTGGATAACCGTATTACCGCCTTTGAGTGAG
CTGATACCGCTCGCCGCAGCCGAACGACCGAGCGCAGCGAGTCAGTGAGCGAGG
AAGCGGAAGAGCGCCCAATACGCAAACCGCCTCTCCCCGCGCGTTGGCCGATTC
ATTAATGCAGCTGGCACGACAGGTTTCCCGACTGGAAAGCGGGCAGTGAGCGCA
ACGCAATTAATACGCGTACCGCTAGCCAGGAAGAGTTTGTAGAAACGCAAAAAG
GCCATCCGTCAGGATGGCCTTCTGCTTAGTTTGATGCCTGGCAGTTTATGGCGGG
CGTCCTGCCCGCCACCCTCCGGGCCGTTGCTTCACAACGTTCAAATCCGCTCCCG
GCGGATTTGTCCTACTCAGGAGAGCGTTCACCGACAAACAACAGATAAAACGAA
AGGCCCAGTCTTCCGACTGAGCCTTTCGTTTTATTTGATGCCTGGCAGTTCCCTAC
TCTCGCGTTAACGCTAGCATGGATGTTTTCCCAGTCACGACGTTGTAAAACGACG
GCCAGTCTTAAGCTCGGGCCCCAAATAATGATTTTATTTTGACTGATAGTGACCT
GTTCGTTGCAACAAATTGATGAGCAATGCTTTTTTATAATGCCAACTTTGTACAA
AAAAGCAGGCTTCTAGACTGCGCGCTCGCTCGCTCACTGAGGCCGCCCGGGCAA
AGCCCGGGCGTCGGGCGACCTTTGGTCGCCCGGCCTCAGTGAGCGAGCGAGCGC
GCAGAGAGGGAGTGGCCAACTCCATCACTAGGGGTTCCTTTAATTAAGGCTCCG
GTGCCCGTCAGTGGGCAGAGCGCACATCGCCCACAGTCCCCGAGAAGTTGGGGG
GAGGGGTCGGCAATTGAACCGGTGCCTAGAGAAGGTGGCGCGGGGTAAACTGGG
AAAGTGATGTCGTGTACTGGCTCCGCCTTTTTCCCGAGGGTGGGGGAGAACCGTA
TATAAGTGCAGTAGTCGCCGTGAACGTTCTTTTTCGCAACGGGTTTGCCGCCAGA
ACACAGGTCAGATCAGATCTTTGTCGATCCTACCATCCACTCGACACACCCGCCA
GCGGCCGCGTTGGTATCAAGGTTACAAGACAGGTTTAAGGAGACCAATAGAAAC
TGGGCATGTGGAGACAGAGAAGACTCTTGGGTTTCTGATAGGCACTGACTCTCTT
CCTTTGTCCTGTTCCCATTTCAGAAGCTTCCGAGCTCTCGAATTAAATAAGAGAG
AAAAGAAGAGTAAGAAGAAATATAAGAGCCACCATGGCCGTGTGTGGACTGGG
CAGCAGACTGGGACTGGGATCTAGACTGGGACTGAGAGGCTGCTTCGGCGCTGC
TAGACTGCTGTACCCTAGATTCCAGTCCAGAGGACCCCAAGGCGTCGAGGATGG
AGATAGACCCCAGCCCTCCTCCAAGACCCCTAGAATCCCCAAGATCTACACCAA
GACCGGCGACAAGGGCTTCAGCTCCACCTTTACCGGCGAGAGGAGACCCAAGGA
CGATCAAGTGTTCGAGGCCGTGGGAACCACCGACGAGCTGAGCTCCGCTATCGG
CTTCGCTCTGGAGCTGGTGACAGAGAAGGGCCACACCTTTGCCGAGGAGCTGCA
GAAGATCCAGTGCACACTGCAAGACGTGGGCAGCGCTCTGGCTACACCTTGCAG
CAGCGCTAGAGAGGCCCATCTGAAATACACCACCTTCAAAGCCGGCCCTATTCTG
GAGCTGGAACAGTGGATCGACAAGTACACCAGCCAGCTGCCTCCTCTGACAGCC
TTCATTCTGCCCAGCGGCGGAAAGATCTCCAGCGCTCTGCACTTCTGCAGAGCCG
TCTGCAGAAGGGCTGAGAGAAGGGTGGTGCCTCTGGTGCAGATGGGCGAGACCG
ATGCCAACGTGGCCAAGTTCCTCAATAGACTGTCCGATTATCTGTTCACACTGGC
TAGATATGCCGCCATGAAGGAGGGCAACCAAGAGAAGATCTACATGAAGAACG
ATCCCAGCGCCGAGAGCGAGGGCCTCTGAACTCGAGATAACAGGCCTATTGATT
GGAAAGTTTGTCAACGAATTGTGGGTCTTTTGGGGTTTGCTGCCCCTTTTACGCA
ATGTGGATATCCTGCTTTAATGCCTTTATATGCATGTATACAAGCAAAACAGGCT
TTTACTTTCTCGCCAACTTACAAGGCCTTTCTCAGTAAACAGTATATGACCCTTTA
CCCCGTTGCTCGGCAACGGCCTGGTCTGTGCCAAGTGTTTGCTGACGCAACCCCC
ACTGGTTGGGGCTTGGCCATAGGCCATCAGCGCATGCGTGGAACCTTTGTGTCTC
CTCTGCCGATCCATACTGCGGAACTCCTAGCCGCTTGTTTTGCTCGCAGCAGGTCT
GGAGCAAACCTCATCGGGACCGACAATTCTGTCGTACTCTCCCGCAAGTATACAT
CGTTTCCATGGCTGCTAGGCTGTGCTGCCAACTGGATCCTGCGCGGGACGTCCTT
TGTTTACGTCCCGTCGGCGCTGAATCCCGCGGACGACCCCTCCCGGGGCCGCTTG
GGGCTCTACCGCCCGCTTCTCCGTCTGCCGTACCGTCCGACCACGGGGCGCACCT
CTCTTTACGCGGACTCCCCGTCTGTGCCTTCTCATCTGCCGGACCGTGTGCACTTC
GCTTCACCTCTGCACGTCGCATGGAGGCCACCGTGAACGCCCACCGGAACCTGCC
CAAGGTCTTGCATAAGAGGACTCTTGGACTTTCAGCAATGTCATCTCCGATCTTTT
TCCCTCTGCCAAAAATTATGGGGACATCATGAAGCCCCTTGAGCATCTGACTTCT
GGCTAATAAAGGAAATTTATTTTCATTGCAATAGTGTGTTGGAATTTTTTGTGTCT
CTCACACTAGTCCACTCCCTCTCTGCGCGCTCGCTCGCTCACTGAGGCCGGGCGA
CCAAAGGTCGCCCGACGCCCGGGCTTTGCCCGGGCGGCCTCAGTGAGCGAGCGA
GCGCGCAGAGAGGGACAGATCCGGGCCCGCATGCGTCGACAATTCACTGGCCGT
CGTTTTACAACGTCGTGACTGGGAAAACCCTGGCGTTACCCAACTTAATCGCCTT
GCAGCACATCCCCCTTTCGCCAGCTGGCGTAATAGCGAAGAGGCCCGCACCGAT
CGCCCTTCCCAACAGTTGCGCAGCCTGAATGGCGAATGGCGCCTGATGCGGTATT
TTCTCCTTACGCATCTGTGCGGTATTTCACACCGCATATGACCCAGCTTTCTTGTA
CAAAGTTGGCATTATAAGAAAGCATTGCTTATCAATTTGTTGCAACGAACAGGTC
ACTATCAGTCAAAATAAAATCATTATTTGCCATCCAGCTGATATCCCCTATAGTG
AGTCGTATTACATGGTCATAGCTGTTTCCTGGCAGCTCTGGCCCGTGTCTCAAAA
TCTCTGATGTTACATTGCACAAGATAAAAATATATCATCATGAACAATAAAACTG
TCTGCTTACATAAACAGTAATACAAGGGGTGTTATGAGCCATATTCAACGGGAA
ACGTCGAGGCCGCGATTAAATTCCAACATGGATGCTGATTTATATGGGTATAAAT
GGGCTCGCGATAATGTCGGGCAATCAGGTGCGACAATCTATCGCTTGTATGGGA
AGCCCGATGCGCCAGAGTTGTTTCTGAAACATGGCAAAGGTAGCGTTGCCAATG
ATGTTACAGATGAGATGGTCAGACTAAACTGGCTGACGGAATTTATGCCTCTTCC
GACCATCAAGCATTTTATCCGTACTCCTGATGATGCATGGTTACTCACCACTGCG
ATCCCCGGAAAAACAGCATTCCAGGTATTAGAAGAATATCCTGATTCAGGTGAA
AATATTGTTGATGCGCTGGCAGTGTTCCTGCGCCGGTTGCATTCGATTCCTGTTTG
TAATTGTCCTTTTAACAGCGATCGCGTATTTCGTCTCGCTCAGGCGCAATCACGA
ATGAATAACGGTTTGGTTGATGCGAGTGATTTTGATGACGAGCGTAATGGCTGGC
CTGTTGAACAAGTCTGGAAAGAAATGCATAAACTTTTGCCATTCTCACCGGATTC
AGTCGTCACTCATGGTGATTTCTCACTTGATAACCTTATTTTTGACGAGGGGAAA
TTAATAGGTTGTATTGATGTTGGACGAGTCGGAATCGCAGACCGATACCAGGATC
TTGCCATCCTATGGAACTGCCTCGGTGAGTTTTCTCCTTCATTACAGAAACGGCTT
TTTCAAAAATATGGTATTGATAATCCTGATATGAATAAATTGCAGTTTCATTTGAT
GCTCGATGAGTTTTTCTAATCAGAATTGGTTAATTGGTTGTAACACTGGCAGAGC
ATTACGCTGACTTGACGGGACGGCGCAAGCTCATGACCAAAATCCCTTAACGTG
AGTTACGCGTCGTTCCACTGAGCGTCAGACCCCGTAGAAAAGATCAAAGGATCTT
CTTGAGATCCTTTTTTTCTGCGCGTAATCTGCTGCTTGCAAACAAAAAAACCACC
GCTACCAGCGGTGGTTTGTTTGCCGGATCAAGAGCTACCAACTCTTTTTCCGAAG
GTAACTGGCTTCAGCAGAGCGCAGATACCAAATACTGTTCTTCTAGTGTAGCCGT
AGTTAGGCCACCACTTCAAGAACTCTGTAGCACCGCCTACATACCTCGCTCTGCT
AATCCTGTTACCAGTGGCTGCTGCCAGTGGCGATAAGTCGTGTCTTACCGGGTTG
GACTCAAGACGATAGTTACCGGATAAGGCGCAGCGGTCGGGCTGAACGGGGGGT
TCGTGCACACAGCCCAGCTTGGAGCGAACGACCTACACCGAACTGAGATACCTA
CAGCGTGAGCTATGAGAAAGCGCCACGCTTCCCGAAGGGAGAAAGGCGGACAG
GTATCCGGTAAGCGGCAGGGTCGGAACAGGAGAGCGCACGAGGGAGCTTCCAG
GGGGAAACGCCTGGTATCTTTATAGTCCTGTCGGGTTTCGCCACCTCTGACTTGA
GCGTCGATTTTTGTGATGCTCGTCAGGGGGGCGGAGCCTATGGAAAAACGCCAG
CAACGCGGCCTTTTTACGGTTCCTGGCCTTTTGCTGGCCTTTTGCTCACATGTT

Amino acid sequence of wild type MMAB polypeptide
(NCBI Reference Sequence: NP_443077.1)
SEQ ID NO: 21
MAVCGLGSRLGLGSRLGLRGCFGAARLLYPRFQSRGPQGVEDGDRPQPS
SKTPRIPKIYTKTGDKGFSSTFTGERRPKDDQVFEAVGTTDELSSAIGF
ALELVTEKGHTFAEELQKIQCTLQDVGSALATPCSSAREAHLKYTTFKA
GPILELEQWIDKYTSQLPPLTAFILPSGGKISSALHFCRAVCRRAERRV
VPLVQMGETDANVAKFLNRLSDYLFTLARYAAMKEGNQEKIYMKNDPSA
ESEGL
Amino acid sequence of wild type MMAB mito-
chondrial transit peptide
SEQ ID NO: 22
MAVCGLGSRLGLGSRLGLRGCFGAARLLYPRF
Amino acid sequence of wild type MMAB mature
enzyme
SEQ ID NO: 23
QSRGPQGVEDGDRPQPSSKTPRIPKIYTKTGDKGFSSTFTGERRPKDDQ
VFEAVGTTDELSSAIGFALELVTEKGHTFAEELQKIQCTLQDVGSALAT
PCSSAREAHLKYTTFKAGPILELEQWIDKYTSQLPPLTAFILPSGGKIS
SALHFCRAVCRRAERRVVPLVQMGETDANVAKFLNRLSDYLFTLARYAA
MKEGNQEKIYMKNDPSAESEGL

### Example 8

This example demonstrates resistance to lethality from dietary challenge conferred by systemic delivery of the inventive vector.

Patient mutations were identified in a large cohort of subjects with MMAB deficiency evaluated enrolled in a natural history study (NCT00078078). One mutation was selected for further modeling in mice: the orthologue of a nonsense mutation frequently found in patients (Q234X, Q228X in mouse) was bred to homozygosity (Mmab^{Q228X/Q228X}) and with mice carrying a null allele (Mmab^{Δ3-7/Q228X}). Various knock-in and compound mutants were characterized and challenged with a diet consisting of a 10-fold increase in Isoleucine and Valine while maintaining total caloric intake. MMAB mice also were treated with a systemic retro-orbital injection of 1e+13 genome copies per kilogram of ssAAV9 EF1L MMAB (SEQ ID NO:19) and examined for response to challenge.

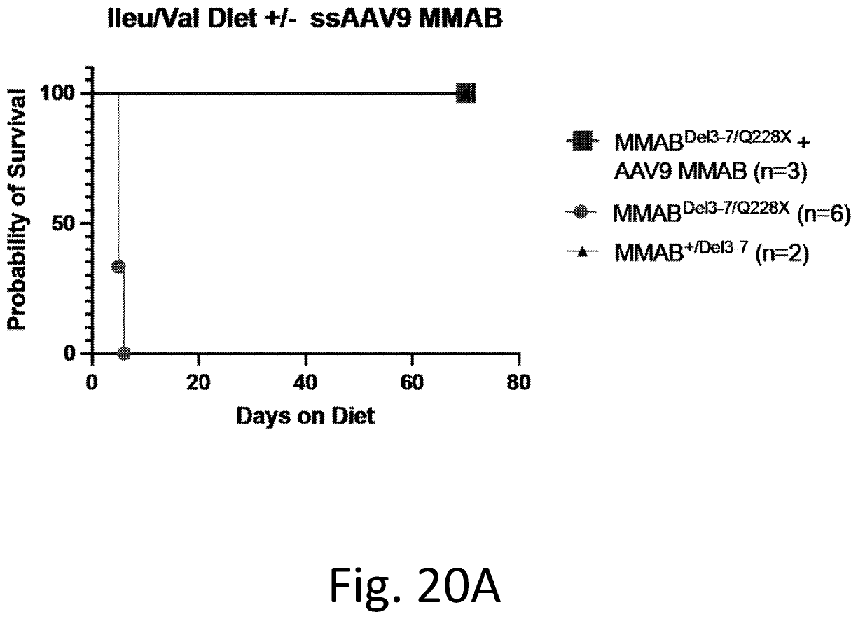

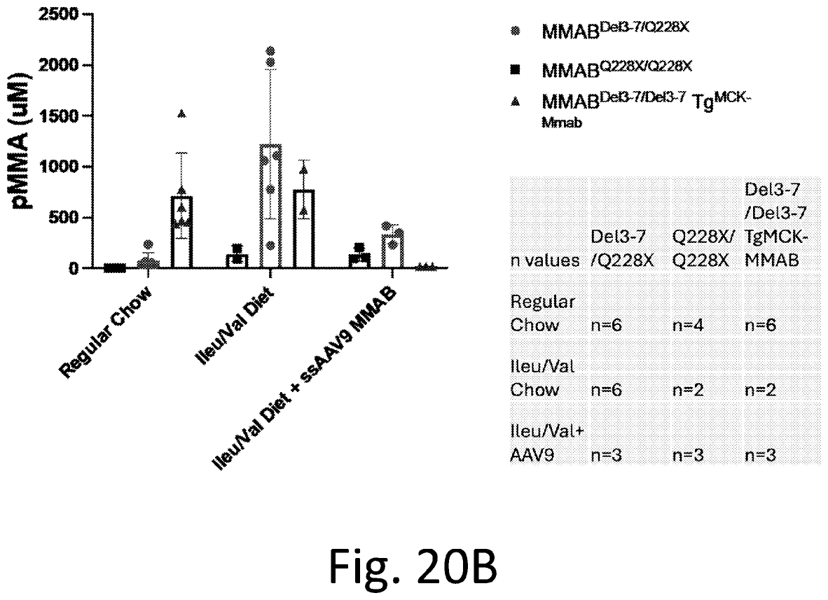

The hypomorphic model, Mmab^{Δ3-7/Q228X}, displayed similar survival and weight gain as compared to wild type controls, while homozygote mutant, Mmab^{Δ3-7/Δ3-7}, showed neonatal lethality. Mmab^{Δ3-7/Q228X} mice displayed mild methylmalonic acidemia [MMA](80 +/−77 μM, ~100× WT), very much like patients with attenuated forms of cb1B MMA. When treated with the isoleucine- and valine-enriched diet, Mmab^{Δ3-7/Q228X} demonstrated significant weight loss (−6.2+/−0.8g n=6 over 4 days) and 100% lethality within 5 days, while controls fed the same diet thrived and gained weight. pMMA levels were drastically increased with the Isoleucine/Valine diet (1223+/−738 μM, ~1500× WT), reflective of severe clinical disease (FIG. 20B). Mmab^{Q228X/Q228X} mice, in contrast, survived the Isoleucine/Valine diet challenge and manifested mild MMAemia (142+/−74 μM) when challenged on the Isoleucine/Valine-enriched diet compared to compound Mmab^{Δ3-7/Q228X} mice.

Treatment of the Mmab^{Δ3-7/Q228X} mice with the ssAAV9.EF1 LMMAB (SEQ ID NO:19), systemically delivered at a dose of 1e13 GC/kg on DOL26, approximately two weeks prior to the high Isoleucine/Valine diet, resulted in complete rescue from lethality (p=0.0081) and reduced weight loss. Additionally, treatment of the Mmab^{Δ3-7/Q228X} mice and Mmab^{Δ3-7/Δ3-7};Tg^{MCK-Mmab} (C57BL/6) mice with the ssAAV9 MMAB: AAV9.EF1 LMMAB Kan vector led to a dramatic reduction in pMMA levels to near those observed for animals not challenged with the high Isoleucine/Valine diet. Data generated by the study reported in this Example are presented in FIGS. 20A and 20B.

These results exemplify the development of a new clinically-relevant murine MMAB mutation and a suite of new murine models to enable preclinical gene therapy studies in adult mice. The Mmab^{Δ3-7/Q228X} mice models can be used in conjunction with an Isoleucine/Valine challenge diet to induce a lethal metabolic crisis, which can be fully rescued from lethality by systemic AAV9 gene therapy using the inventive vectors. This model demonstrates that AAV9 gene therapy can be used to protect against acute lethal metabolic instability, allowing the facile assessment of vector efficacy and dose finding in the development of new gene therapeutics for patients with MMAB deficiency.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A synthetic MMAB (Metabolism Of Cobalamin Associated B) polynucleotide (synMMAB) selected from the group consisting of:
(a) a polynucleotide comprising the nucleic acid sequence of any one of SEQ ID NOs: 1-6;
(b) a polynucleotide having a nucleic acid sequence with at least about 80% identity to the nucleic acid sequence of any one of SEQ ID NOs: 1-6 and encoding a polypeptide having an amino acid sequence at least about 90% identical to SEQ ID NO: 21, and having equivalent or increased expression in a mammalian host cell relative to expression of SEQ ID NO: 7 in the same host cell, wherein the polynucleotide does not have the nucleic acid sequence of SEQ ID NO: 7; and
(c) a polynucleotide having a nucleic acid sequence with at least about 80% identity to the nucleic acid sequence of nucleotides 97-753 of any one of SEQ ID NOs: 1-6 and encoding a polypeptide having an amino acid sequence at least about 90% identical to SEQ ID NO: 23, and having equivalent or increased expression in a mammalian host cell relative to expression of nucleotides 97-753 of SEQ ID NO: 7 in the same host cell, wherein the polynucleotide does not have the nucleic acid sequence of nucleotides 97-753 of SEQ ID NO:
7.

2. The synthetic polynucleotide of claim 1, wherein the polynucleotide has (a) at least about 90% identity to the nucleic acid sequence of any one of SEQ ID NOs: 1-6 or (b) at least about 90% identity to the nucleic acid sequence of nucleotides 97-753 of any one of SEQ ID NOs: 1-6.

3. The synthetic polynucleotide of claim 1, wherein the polynucleotide has (a) at least about 95% identity to the nucleic acid sequence of any one of SEQ ID NOs: 1-6 or (b) at least about 95% identity to the nucleic acid sequence of nucleotides 97-753 of any one of SEQ ID NOs: 1-6.

4. The synthetic polynucleotide of claim 1, wherein the polynucleotide exhibits increased expression in a mammalian host cell relative to the expression SEQ ID NO: 7 or nucleotides 97-753 of SEQ ID NO: 7 in the mammalian host cell.

5. The synthetic polynucleotide of claim 1, wherein the synthetic polynucleotide comprises codons that have been optimized relative to the naturally occurring human MMAB polynucleotide sequence of SEQ ID NO: 7 or nucleotides 97-753 of SEQ ID NO: 7.

6. The synthetic polynucleotide of claim 5, wherein the nucleic acid sequence has at least about 70% of less commonly used codons replaced with more commonly used codons.

7. A recombinant expression vector configured for expression of the synthetic polynucleotide of claim 1.

8. The recombinant expression vector of claim 7, wherein the recombinant expression vector is single stranded.

9. The recombinant expression vector of claim 7, wherein the recombinant expression vector is self-complementary.

10. The recombinant expression vector of claim 7, wherein the recombinant expression vector is a viral vector.

11. The recombinant expression vector of claim 7, wherein the recombinant expression vector is an adeno-associated viral (AAV) vector.

12. The recombinant expression vector of claim 7, wherein the recombinant expression vector is an AAV8 or AAV9 vector.

13. The recombinant expression vector of claim 7, wherein the recombinant expression vector is a non-viral vector.

14. The recombinant expression vector of claim 7, wherein the recombinant expression vector is configured for expression of the synMMAB polynucleotide in one or both of hepatic tissue and renal tissue.

15. The recombinant expression vector of claim 7, wherein the recombinant expression vector comprises the nucleic acid sequence of any one of SEQ ID NOs: 9-10 and 13-20.

16. An isolated or purified host cell comprising the recombinant expression vector of claim 7.

17. An isolated or purified population of cells comprising the host cell of claim 16.

18. A pharmaceutical composition comprising (i) the synthetic polynucleotide of claim 1, a recombinant expression vector configured for expression thereof, a host cell comprising the recombinant expression vector or a population of such cells and (ii) a pharmaceutically acceptable carrier.

19. A method of treating a disease or condition mediated by MMAB (Metabolism Of Cobalamin Associated B) enzyme, comprising administering to a mammal in need thereof a therapeutic amount the pharmaceutical composition of claim 18.

20. A method of treating a disease or condition mediated by MMAB (Metabolism Of Cobalamin Associated B) enzyme, comprising:
producing the MMAB (Metabolism Of Cobalamin Associated B) enzyme by expressing the synthetic polynucleotide of claim 1 by a host cell, and purifying the enzyme from the host cell; and
administering to a mammal in need thereof the purified MMAB (Metabolism Of Cobalamin Associated B) enzyme.

21. A method of treating a disease or condition mediated by MMAB (Metabolism Of Cobalamin Associated B) enzyme, comprising administering to a cell of a mammal in need thereof the polynucleotide of claim 1, wherein the polynucleotide is inserted into the cell of the mammal via genome editing on the cell of the mammal using a nuclease selected from the group of zinc finger nucleases (ZFNs), transcription activator-like effector nucleases (TALENs), the clustered regularly interspaced short palindromic repeats (CRISPR/cas system) and meganuclease re-engineered homing endonucleases on a cell from the mammal.

22. The method of claim 21, wherein the method comprises administering the polynucleotide to an isolated cell of the mammal, and the method further comprises administering the cell to the mammal.

23. The method of claim 21, wherein the method comprises administering the polynucleotide to the cell of the mammal in vivo.

24. The method of claim 19, wherein the disease or condition is methylmalonic acidemia (MMA).

25. The method of claim 19, wherein the disease or condition is MMAB deficiency.

26. A method of detecting the presence of a synthetic MMAB (Metabolism Of Cobalamin Associated B) polynucleotide (synMAB) in a biological sample from a mammal, the method comprising:
(a) obtaining at least one test sample comprising nucleic acid from a biological sample from a mammal;
(b) contacting any of the synthetic polynucleotide of claim 1 with the at least one test sample under conditions allowing for a complex to form between the synthetic polynucleotide and the isolated nucleic acid of the test sample;
(c) detecting the complex; and
(d) comparing a presence of the complex in the at least one test sample with an absence of complex from a negative sample that lacks the synthetic polynucleotide, wherein detection of the complex is indicative of the presence of the synthetic polynucleotide in the biological sample from the mammal.


SEQUENCE LISTING (23 sequences)

SEQ ID NO: 1 - DNA, 753 bp, synthetic construct
  source 1..753: mol_type = other DNA
atggctgtctgcggtctgggcagcagactgggtcttgggtcccgcttgggactccgaggctgctttggtgcagcacgcttgctgtatcctcgcttccagtctagagggccacaaggagtagaggacggtgatcggccccagccaagcagcaaaacaccgaggataccgaaaatttatacaaagacgggagataaggggtttagcagcaccttcacgggcgagcgacggcccaaggacgaccaagtattcgaggcagtcgggacgacggacgaactgtcaagtgctattgggttcgctctggagctggttacggagaagggacatactttcgcggaggagctccaaaaaatacaatgcacccttcaagacgtgggcagtgccctggcgacaccgtgtagcagcgcgcgcgaggcccacttgaaatatacaactttcaaagcaggacctatattggaactcgagcagtggatagacaaatacacgtcccaactgcctcccctgacagcttttatcttgccatctggcggtaagataagttctgctcttcatttctgtagggcagtatgtaggcgagcggaaaggcgggttgtacccctggtgcagatgggtgagactgacgccaatgtggccaagttcttgaaccgcctgtctgattatctgtttacactcgctagatacgccgctatgaaagaaggaaatcaagagaagatatacatgaagaacgatccgtcagcggaatccgaggggctctga

SEQ ID NO: 2 - DNA, 753 bp, synthetic construct
  source 1..753: mol_type = other DNA
atggcggtctgcggattgggttcacgattgggccttgggtctaggctcggcctgcgaggttgtttcggagctgctagattgttgtacccacgattccaaagcaggggaccacaaggtgtggaagacggtgacaggcctcagccctcaagcaaaacccctcggattccgaaaatatatacaaagacaggagacaaaggcttttcaagcacgtttactggcgaacgaaggcctaaagacgatcaagtttttgaggcggtagggacaactgacgaactttcatctgcgataggcttcgcgctcgagttggtgacggagaagggtcatacattcgctgaagaacttcagaaaattcagtgtacgctccaagatgttggcagtgcgcttgcgacaccttgcagctcagcaagggaagcgcatttgaagtacaccactttcaaagcgggcccgattctcgaacttgaacaatggattgacaaatatacaagccaactgccgcctcttaccgccttcatcctccctagtggtggcaaaatttcttccgcgcttcatttttgtagggccgtgtgtcgccgcgccgagagacgggtggttccgttggtacagatgggtgaaacagatgcaaacgtggcaaagttccttaaccgcctcagcgactacttgttcactctcgctaggtatgccgccatgaaagaagggaatcaagagaagatctatatgaagaatgacccgagcgctgaaagcgagggactttga

SEQ ID NO: 3 - DNA, 753 bp, synthetic construct
  source 1..753: mol_type = other DNA
atggccgtgtgcggcctgggcagcagactgggactgggttcccggctgggactgagaggctgcttcggcgccgcaagactgctgtaccccagattccagagcagaggacctcagggcgtggaagatggcgatagacctcaacctagctcaaagacccctagaatccccaagatctacacaaagacaggcgacaagggctttagcagcacctttaccggcgaaagacggcctaaggacgaccaggttttcgaggccgtcggaacaaccgacgagctgagctctgctatcggcttcgccctcgagctcgtgaccgagaagggccacacatttgccgaggaactgcaaaaaattcagtgtaccctgcaggacgtgggatctgctctggccacaccttgcagcagcgctcgggaagcccacctgaagtacaccaccttcaaggccggcccaatcctggaactggaacagtggatcgacaagtacaccagccagctgccacctctgaccgccttcatcctgccttctggcggcaagatcagcagcgccctgcacttctgcagagccgtgtgtagaagggccgagcggcgggtggtgcccctggtgcagatgggcgagacagatgccaacgtggccaagttcctgaatagactgtctgattacctgttcaccctggctcgctacgccgccatgaaggagggcaaccaggagaaaatctatatgaaaaacgacccctccgccgagagcgagggcctgtga

SEQ ID NO: 4 - DNA, 753 bp, synthetic construct
  source 1..753: mol_type = other DNA
atggccgtttgtggcctgggctctagactgggactgggctcccggctcggactgagaggatgttttggcgccgctagactgctgtaccccagattccagagccggggccctcagggcgtggaagatggcgatagacctcaacctagcagcaagacccctagaatccctaagatctacacaaagaccggcgataagggcttcagctccaccttcacaggcgaaagaagacctaaggacgaccaggtgttcgaggccgtgggcaccaccgacgagctgagctccgctatcggatttgccctggagctggtgacagagaagggccacaccttcgccgaagagctgcaaaagattcagtgcaccctgcaggacgtgggcagcgccctggccaccccatgcagcagcgcacgggaagcccacctgaagtacacaacctttaaggccggacctatcctggaactggaacagtggatcgacaaatacaccagccagctgccccccctcacagccttcatcctgccaagcggcggcaagatcagctctgctctgcacttctgcagagccgtgtgccggcgggccgagagaagggtggtccccctggtgcagatgggcgagacagacgccaatgtggccaagttcctgaaccgcctgtctgattacctgttcaccctggccagatacgccgccatgaaggaaggcaaccaggagaaaatctatatgaaaaacgacccttctgctgagagcgaggggctgtga

SEQ ID NO: 5 - DNA, 753 bp, synthetic construct
  source 1..753: mol_type = other DNA
atggccgtgtgtggactgggcagcagactgggactgggatctagactgggactgagaggctgcttcggcgctgctagactgctgtaccctagattccagtccagaggaccccaaggcgtcgaggatggagatagaccccagccctcctccaagacccctagaatccccaagatctacaccaagaccggcgacaagggcttcagctccacctttaccggcgagaggagacccaaggacgatcaagtgttcgaggccgtgggaaccaccgacgagctgagctccgctatcggcttcgctctggagctggtgacagagaagggccacacctttgccgaggagctgcagaagatccagtgcacactgcaagacgtgggcagcgctctggctacaccttgcagcagcgctagagaggcccatctgaaatacaccaccttcaaagccggccctattctggagctggaacagtggatcgacaagtacaccagccagctgcctcctctgacagccttcattctgcccagcggcggaaagatctccagcgctctgcacttctgcagagccgtctgcagaagggctgagagaagggtggtgcctctggtgcagatgggcgagaccgatgccaacgtggccaagttcctcaatagactgtccgattatctgttcacactggctagatatgccgccatgaaggagggcaaccaagagaagatctacatgaagaacgatcccagcgccgagagcgagggcctctga

SEQ ID NO: 6 - DNA, 753 bp, synthetic construct
  source 1..753: mol_type = other DNA
atggccgtgtgtggactgggcagcagactgggactgggcagcagactgggactgagaggatgctttggcgccgctagactgctgtaccctagattccagtctagaggccctcaaggcgtggaggatggagacagaccccagcccagctccaaaacccctagaatccccaagatctacaccaagaccggcgacaagggcttcagcagcaccttcaccggcgagagaagacccaaggatgaccaagtgttcgaggccgtgggcaccaccgacgaactgtccagcgccattggcttcgctctggagctggtgaccgagaagggacacacctttgccgaggagctgcagaagattcagtgcacactgcaagacgtgggcagcgctctggccaccccttgcagctccgctagggaagcccatctgaagtacaccaccttcaaggccggccctattctggagctcgagcagtggatcgacaaatacaccagccagctgcctcctctgaccgccttcattctgccttccggaggcaagatctccagcgctctgcacttctgtagagccgtgtgtagaagagccgaaagaagagtggtccctctggtgcagatgggagagaccgacgccaacgtggccaagtttctgaatagactgagcgattatctgttcacactggctagatacgccgccatgaaggagggcaaccaagagaagatctacatgaagaacgacccctccgccgagagcgaaggactgtga

SEQ ID NO: 7 - DNA, 753 bp, Homo sapiens
  source 1..753: mol_type = genomic DNA
atggctgtgtgcggcctggggagccgtcttggcctggggagccgtcttggcctgcgcgggtgcttcggcgccgccaggctcctgtatccccgtttccagagccgcggccctcagggcgtggaagacggggacaggccacagccttcctcgaagacacccaggatccccaagatttacaccaaaacgggagacaaagggttttctagtaccttcacaggagaaaggagacccaaagatgaccaagtgtttgaagccgtgggaactacagatgaattaagttcagctattgggtttgctctggaattagtcacagaaaagggccatacatttgccgaagagcttcagaaaatccagtgcacattgcaggacgtcggctcggccctggcgacaccatgctcctcggcccgggaggctcacttaaagtataccacgttcaaggcggggcccatcctggagctggagcagtggatcgacaagtacaccagccagctcccaccactcacggccttcatcctgccttcgggaggcaagatcagctcggcgctgcatttctgccgggccgtgtgccgccgggccgagagacgtgtggtgcctcttgtccagatgggagagaccgatgcgaacgtggccaagttcttaaacagactcagtgactatctcttcacgctagccagatatgcagccatgaaggaggggaatcaagagaaaatatacatgaaaaatgacccatcggccgagtctgagggactctga

SEQ ID NO: 8 - DNA, 6124 bp, synthetic construct
  source 1..6124: mol_type = other DNA
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggcttctagactgcgcgctcgctcgctcactgaggccgcccgggcaaagcccgggcgtcgggcgacctttggtcgcccggcctcagtgagcgagcgagcgcgcagagagggagtggccaactccatcactaggggttcctttaattaatacgtaggtaccggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtactgaattcatggctgtgtgcggcctggggagccgtcttggcctggggagccgtcttggcctgcgcgggtgcttcggcgccgccaggctcctgtatccccgtttccagagccgcggccctcagggcgtggaagacggggacaggccacagccttcctcgaagacacccaggatccccaagatttacaccaaaacgggagacaaagggttttctagtaccttcacaggagaaaggagacccaaagatgaccaagtgtttgaagccgtgggaactacagatgaattaagttcagctattgggtttgctctggaattagtcacagaaaagggccatacatttgccgaagagcttcagaaaatccagtgcacattgcaggacgtcggctcggccctggcgacaccatgctcctcggcccgggaggctcacttaaagtataccacgttcaaggcggggcccatcctggagctggagcagtggatcgacaagtacaccagccagctcccaccactcacggccttcatcctgccttcgggaggcaagatcagctcggcgctgcatttctgccgggccgtgtgccgccgggccgagagacgtgtggtgcctcttgtccagatgggagagaccgatgcgaacgtggccaagttcttaaacagactcagtgactatctcttcacgctagccagatatgcagccatgaaggaggggaatcaagagaaaatatacatgaaaaatgacccatcggccgagtctgagggactctgaggcctcgagataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatcgatatcgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagactagtcctgcaggtacgtaagcggccgcggcctaggaacccctagtgatggagttggccactccctctctgcgcgctcgctcgctcactgaggccgggcgaccaaaggtcgcccgacgcccgggctttgcccgggcggcctcagtgagcgagcgagcgcgcagcatatgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 9 - DNA, 6112 bp, synthetic construct
  source 1..6112: mol_type = other DNA
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagctgggtcatatgctgcgcgctcgctcgctcactgaggccgcccgggcaaagcccgggcgtcgggcgacctttggtcgcccggcctcagtgagcgagcgagcgcgcagagagggagtggccaactccatcactaggggttcctttaattaaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtactgaattcatggctgtctgcggtctgggcagcagactgggtcttgggtcccgcttgggactccgaggctgctttggtgcagcacgcttgctgtatcctcgcttccagtctagagggccacaaggagtagaggacggtgatcggccccagccaagcagcaaaacaccgaggataccgaaaatttatacaaagacgggagataaggggtttagcagcaccttcacgggcgagcgacggcccaaggacgaccaagtattcgaggcagtcgggacgacggacgaactgtcaagtgctattgggttcgctctggagctggttacggagaagggacatactttcgcggaggagctccaaaaaatacaatgcacccttcaagacgtgggcagtgccctggcgacaccgtgtagcagcgcgcgcgaggcccacttgaaatatacaactttcaaagcaggacctatattggaactcgagcagtggatagacaaatacacgtcccaactgcctcccctgacagcttttatcttgccatctggcggtaagataagttctgctcttcatttctgtagggcagtatgtaggcgagcggaaaggcgggttgtacccctggtgcagatgggtgagactgacgccaatgtggccaagttcttgaaccgcctgtctgattatctgtttacactcgctagatacgccgctatgaaagaaggaaatcaagagaagatatacatgaagaacgatccgtcagcggaatccgaggggctctgaggcctcgagataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatcgatatcgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagactagtcctgcaggtacgtaagcggccgcggcctaggaacccctagtgatggagttggccactccctctctgcgcgctcgctcgctcactgaggccgggcgaccaaaggtcgcccgacgcccgggctttgcccgggcggcctcagtgagcgagcgagcgcgcagcatatgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 10 - DNA, 6112 bp, synthetic construct
  source 1..6112: mol_type = other DNA
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggcttctagactgcgcgctcgctcgctcactgaggccgcccgggcaaagcccgggcgtcgggcgacctttggtcgcccggcctcagtgagcgagcgagcgcgcagagagggagtggccaactccatcactaggggttcctttaattaaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtactgaattcatggcggtctgcggattgggttcacgattgggccttgggtctaggctcggcctgcgaggttgtttcggagctgctagattgttgtacccacgattccaaagcaggggaccacaaggtgtggaagacggtgacaggcctcagccctcaagcaaaacccctcggattccgaaaatatatacaaagacaggagacaaaggcttttcaagcacgtttactggcgaacgaaggcctaaagacgatcaagtttttgaggcggtagggacaactgacgaactttcatctgcgataggcttcgcgctcgagttggtgacggagaagggtcatacattcgctgaagaacttcagaaaattcagtgtacgctccaagatgttggcagtgcgcttgcgacaccttgcagctcagcaagggaagcgcatttgaagtacaccactttcaaagcgggcccgattctcgaacttgaacaatggattgacaaatatacaagccaactgccgcctcttaccgccttcatcctccctagtggtggcaaaatttcttccgcgcttcatttttgtagggccgtgtgtcgccgcgccgagagacgggtggttccgttggtacagatgggtgaaacagatgcaaacgtggcaaagttccttaaccgcctcagcgactacttgttcactctcgctaggtatgccgccatgaaagaagggaatcaagagaagatctatatgaagaatgacccgagcgctgaaagcgagggactttgaggcctcgagataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatcgatatcgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagactagtcctgcaggtacgtaagcggccgcggcctaggaacccctagtgatggagttggccactccctctctgcgcgctcgctcgctcactgaggccgggcgaccaaaggtcgcccgacgcccgggctttgcccgggcggcctcagtgagcgagcgagcgcgcagcatatgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 11 - DNA, 5216 bp, synthetic construct
  source 1..5216: mol_type = other DNA
ggccactccctctctgcgcgctcgctcgctcactgaggccgggcgaccaaaggtcgcccggcctcagtgagcgagcgagcgcgcagagagggagtggccaactccatcactaggggttcctggaggggtggagtcgtgacctaggggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtcagatcagatctttgtcgatcctaccatccactcgacacacccgccagcggccgcgttggtatcaaggttacaagacaggtttaaggagaccaatagaaactgggcatgtggagacagagaagactcttgggtttctgataggcactgactctcttcctttgtcctgttcccatttcagaagcttccgagctctcgaattaaataagagagaaaagaagagtaagaagaaatataagagccaccatggctgtgtgcggcctggggagccgtcttggcctggggagccgtcttggcctgcgcgggtgcttcggcgccgccaggctcctgtatccccgtttccagagccgcggccctcagggcgtggaagacggggacaggccacagccttcctcgaagacacccaggatccccaagatttacaccaaaacgggagacaaagggttttctagtaccttcacaggagaaaggagacccaaagatgaccaagtgtttgaagccgtgggaactacagatgaattaagttcagctattgggtttgctctggaattagtcacagaaaagggccatacatttgccgaagagcttcagaaaatccagtgcacattgcaggacgtcggctcggccctggcgacaccatgctcctcggcccgggaggctcacttaaagtataccacgttcaaggcggggcccatcctggagctggagcagtggatcgacaagtacaccagccagctcccaccactcacggccttcatcctgccttcgggaggcaagatcagctcggcgctgcatttctgccgggccgtgtgccgccgggccgagagacgtgtggtgcctcttgtccagatgggagagaccgatgcgaacgtggccaagttcttaaacagactcagtgactatctcttcacgctagccagatatgcagccatgaaggaggggaatcaagagaaaatatacatgaaaaatgacccatcggccgagtctgagggactctgaactcgagataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatcgatatcgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagaactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctagaaagcttcccggggggatctg

SEQ ID NO: 12 - DNA, 5051 bp, synthetic construct
  source 1..5051: mol_type = other DNA
ggccactccctctctgcgcgctcgctcgctcactgaggccgggcgaccaaaggtcgcccggcctcagtgagcgagcgagcgcgcagagagggagtggccaactccatcactaggggttcctggaggggtggagtcgtgacctaggggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtcagatcagatctttgtcgatcctaccatccactcgacacacccgccagcggccgcgttggtatcaaggttacaagacaggtttaaggagaccaatagaaactgggcatgtggagacagagaagactcttgggtttctgataggcactgactctcttcctttgtcctgttcccatttcagaagcttccgagctctcgaattaaataagagagaaaagaagagtaagaagaaatataagagccaccatggctgtgtgcggcctggggagccgtcttggcctggggagccgtcttggcctgcgcgggtgcttcggcgccgccaggctcctgtatccccgtttccagagccgcggccctcagggcgtggaagacggggacaggccacagccttcctcgaagacacccaggatccccaagatttacaccaaaacgggagacaaagggttttctagtaccttcacaggagaaaggagacccaaagatgaccaagtgtttgaagccgtgggaactacagatgaattaagttcagctattgggtttgctctggaattagtcacagaaaagggccatacatttgccgaagagcttcagaaaatccagtgcacattgcaggacgtcggctcggccctggcgacaccatgctcctcggcccgggaggctcacttaaagtataccacgttcaaggcggggcccatcctggagctggagcagtggatcgacaagtacaccagccagctcccaccactcacggccttcatcctgccttcgggaggcaagatcagctcggcgctgcatttctgccgggccgtgtgccgccgggccgagagacgtgtggtgcctcttgtccagatgggagagaccgatgcgaacgtggccaagttcttaaacagactcagtgactatctcttcacgctagccagatatgcagccatgaaggaggggaatcaagagaaaatatacatgaaaaatgacccatcggccgagtctgagggactctgaactcgagataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatctccgatctttttccctctgccaaaaattatggggacatcatgaagccccttgagcatctgacttctggctaataaaggaaatttattttcattgcaatagtgtgttggaattttttgtgtctctcacactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctagaaagcttcccggggggatctg

SEQ ID NO: 13 - DNA, 5051 bp, synthetic construct
  source 1..5051: mol_type = other DNA
ggccactccctctctgcgcgctcgctcgctcactgaggccgggcgaccaaaggtcgcccggcctcagtgagcgagcgagcgcgcagagagggagtggccaactccatcactaggggttcctggaggggtggagtcgtgacctaggggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtcagatcagatctttgtcgatcctaccatccactcgacacacccgccagcggccgcgttggtatcaaggttacaagacaggtttaaggagaccaatagaaactgggcatgtggagacagagaagactcttgggtttctgataggcactgactctcttcctttgtcctgttcccatttcagaagcttccgagctctcgaattaaataagagagaaaagaagagtaagaagaaatataagagccaccatggctgtctgcggtctgggcagcagactgggtcttgggtcccgcttgggactccgaggctgctttggtgcagcacgcttgctgtatcctcgcttccagtctagagggccacaaggagtagaggacggtgatcggccccagccaagcagcaaaacaccgaggataccgaaaatttatacaaagacgggagataaggggtttagcagcaccttcacgggcgagcgacggcccaaggacgaccaagtattcgaggcagtcgggacgacggacgaactgtcaagtgctattgggttcgctctggagctggttacggagaagggacatactttcgcggaggagctccaaaaaatacaatgcacccttcaagacgtgggcagtgccctggcgacaccgtgtagcagcgcgcgcgaggcccacttgaaatatacaactttcaaagcaggacctatattggaactcgagcagtggatagacaaatacacgtcccaactgcctcccctgacagcttttatcttgccatctggcggtaagataagttctgctcttcatttctgtagggcagtatgtaggcgagcggaaaggcgggttgtacccctggtgcagatgggtgagactgacgccaatgtggccaagttcttgaaccgcctgtctgattatctgtttacactcgctagatacgccgctatgaaagaaggaaatcaagagaagatatacatgaagaacgatccgtcagcggaatccgaggggctctgaactcgagataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatctccgatctttttccctctgccaaaaattatggggacatcatgaagccccttgagcatctgacttctggctaataaaggaaatttattttcattgcaatagtgtgttggaattttttgtgtctctcacactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctagaaagcttcccggggggatctg

SEQ ID NO: 14 - DNA, 5019 bp, synthetic construct
  source 1..5019: mol_type = other DNA
ggccactccctctctgcgcgctcgcccggcctcagtgagcgagcgagcgcgcagagagggagtggccaactccatcactaggggttcctggaggggtggagtcgtgacctaggggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtcagatcagatctttgtcgatcctaccatccactcgacacacccgccagcggccgcgttggtatcaaggttacaagacaggtttaaggagaccaatagaaactgggcatgtggagacagagaagactcttgggtttctgataggcactgactctcttcctttgtcctgttcccatttcagaagcttccgagctctcgaattaaataagagagaaaagaagagtaagaagaaatataagagccaccatggcggtctgcggattgggttcacgattgggccttgggtctaggctcggcctgcgaggttgtttcggagctgctagattgttgtacccacgattccaaagcaggggaccacaaggtgtggaagacggtgacaggcctcagccctcaagcaaaacccctcggattccgaaaatatatacaaagacaggagacaaaggcttttcaagcacgtttactggcgaacgaaggcctaaagacgatcaagtttttgaggcggtagggacaactgacgaactttcatctgcgataggcttcgcgctcgagttggtgacggagaagggtcatacattcgctgaagaacttcagaaaattcagtgtacgctccaagatgttggcagtgcgcttgcgacaccttgcagctcagcaagggaagcgcatttgaagtacaccactttcaaagcgggcccgattctcgaacttgaacaatggattgacaaatatacaagccaactgccgcctcttaccgccttcatcctccctagtggtggcaaaatttcttccgcgcttcatttttgtagggccgtgtgtcgccgcgccgagagacgggtggttccgttggtacagatgggtgaaacagatgcaaacgtggcaaagttccttaaccgcctcagcgactacttgttcactctcgctaggtatgccgccatgaaagaagggaatcaagagaagatctatatgaagaatgacccgagcgctgaaagcgagggactttgaactcgagataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatctccgatctttttccctctgccaaaaattatggggacatcatgaagccccttgagcatctgacttctggctaataaaggaaatttattttcattgcaatagtgtgttggaattttttgtgtctctcacactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctagaaagcttcccggggggatctg

SEQ ID NO: 15 - DNA, 5051 bp, synthetic construct
  source 1..5051: mol_type = other DNA
ggccactccctctctgcgcgctcgctcgctcactgaggccgggcgaccaaaggtcgcccggcctcagtgagcgagcgagcgcgcagagagggagtggccaactccatcactaggggttcctggaggggtggagtcgtgacctaggggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtcagatcagatctttgtcgatcctaccatccactcgacacacccgccagcggccgcgttggtatcaaggttacaagacaggtttaaggagaccaatagaaactgggcatgtggagacagagaagactcttgggtttctgataggcactgactctcttcctttgtcctgttcccatttcagaagcttccgagctctcgaattaaataagagagaaaagaagagtaagaagaaatataagagccaccatggccgtgtgcggcctgggcagcagactgggactgggttcccggctgggactgagaggctgcttcggcgccgcaagactgctgtaccccagattccagagcagaggacctcagggcgtggaagatggcgatagacctcaacctagctcaaagacccctagaatccccaagatctacacaaagacaggcgacaagggctttagcagcacctttaccggcgaaagacggcctaaggacgaccaggttttcgaggccgtcggaacaaccgacgagctgagctctgctatcggcttcgccctcgagctcgtgaccgagaagggccacacatttgccgaggaactgcaaaaaattcagtgtaccctgcaggacgtgggatctgctctggccacaccttgcagcagcgctcgggaagcccacctgaagtacaccaccttcaaggccggcccaatcctggaactggaacagtggatcgacaagtacaccagccagctgccacctctgaccgccttcatcctgccttctggcggcaagatcagcagcgccctgcacttctgcagagccgtgtgtagaagggccgagcggcgggtggtgcccctggtgcagatgggcgagacagatgccaacgtggccaagttcctgaatagactgtctgattacctgttcaccctggctcgctacgccgccatgaaggagggcaaccaggagaaaatctatatgaaaaacgacccctccgccgagagcgagggcctgtgaactcgagataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatctccgatctttttccctctgccaaaaattatggggacatcatgaagccccttgagcatctgacttctggctaataaaggaaatttattttcattgcaatagtgtgttggaattttttgtgtctctcacactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctagaaagcttcccggggggatctg

SEQ ID NO: 16 - DNA, 5051 bp, synthetic construct
  source 1..5051: mol_type = other DNA
ggccactccctctctgcgcgctcgctcgctcactgaggccgggcgacctttggtcgcccggcctcagtgagcgagcgagcgcgcagagagggagtggccaactccatcactaggggttcctggaggggtggagtcgtgacctaggggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtcagatcagatctttgtcgatcctaccatccactcgacacacccgccagcggccgcgttggtatcaaggttacaagacaggtttaaggagaccaatagaaactgggcatgtggagacagagaagactcttgggtttctgataggcactgactctcttcctttgtcctgttcccatttcagaagcttccgagctctcgaattaaataagagagaaaagaagagtaagaagaaatataagagccaccatggccgtttgtggcctgggctctagactgggactgggctcccggctcggactgagaggatgttttggcgccgctagactgctgtaccccagattccagagccggggccctcagggcgtggaagatggcgatagacctcaacctagcagcaagacccctagaatccctaagatctacacaaagaccggcgataagggcttcagctccaccttcacaggcgaaagaagacctaaggacgaccaggtgttcgaggccgtgggcaccaccgacgagctgagctccgctatcggatttgccctggagctggtgacagagaagggccacaccttcgccgaagagctgcaaaagattcagtgcaccctgcaggacgtgggcagcgccctggccaccccatgcagcagcgcacgggaagcccacctgaagtacacaacctttaaggccggacctatcctggaactggaacagtggatcgacaaatacaccagccagctgccccccctcacagccttcatcctgccaagcggcggcaagatcagctctgctctgcacttctgcagagccgtgtgccggcgggccgagagaagggtggtccccctggtgcagatgggcgagacagacgccaatgtggccaagttcctgaaccgcctgtctgattacctgttcaccctggccagatacgccgccatgaaggaaggcaaccaggagaaaatctatatgaaaaacgacccttctgctgagagcgaggggctgtgaactcgagataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatctccgatctttttccctctgccaaaaattatggggacatcatgaagccccttgagcatctgacttctggctaataaaggaaatttattttcattgcaatagtgtgttggaattttttgtgtctctcacactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctagaaagcttcccggggggatctg

SEQ ID NO: 17 - DNA, 5051 bp, synthetic construct
  source 1..5051: mol_type = other DNA
ggccactccctctctgcgcgctcgctcgctcactgaggccgggcgaccaaaggtcgcccggcctcagtgagcgagcgagcgcgcagagagggagtggccaactccatcactaggggttcctggaggggtggagtcgtgacctaggggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtcagatcagatctttgtcgatcctaccatccactcgacacacccgccagcggccgcgttggtatcaaggttacaagacaggtttaaggagaccaatagaaactgggcatgtggagacagagaagactcttgggtttctgataggcactgactctcttcctttgtcctgttcccatttcagaagcttccgagctctcgaattaaataagagagaaaagaagagtaagaagaaatataagagccaccatggccgtgtgtggactgggcagcagactgggactgggatctagactgggactgagaggctgcttcggcgctgctagactgctgtaccctagattccagtccagaggaccccaaggcgtcgaggatggagatagaccccagccctcctccaagacccctagaatccccaagatctacaccaagaccggcgacaagggcttcagctccacctttaccggcgagaggagacccaaggacgatcaagtgttcgaggccgtgggaaccaccgacgagctgagctccgctatcggcttcgctctggagctggtgacagagaagggccacacctttgccgaggagctgcagaagatccagtgcacactgcaagacgtgggcagcgctctggctacaccttgcagcagcgctagagaggcccatctgaaatacaccaccttcaaagccggccctattctggagctggaacagtggatcgacaagtacaccagccagctgcctcctctgacagccttcattctgcccagcggcggaaagatctccagcgctctgcacttctgcagagccgtctgcagaagggctgagagaagggtggtgcctctggtgcagatgggcgagaccgatgccaacgtggccaagttcctcaatagactgtccgattatctgttcacactggctagatatgccgccatgaaggagggcaaccaagagaagatctacatgaagaacgatcccagcgccgagagcgagggcctctgaactcgagataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatctccgatctttttccctctgccaaaaattatggggacatcatgaagccccttgagcatctgacttctggctaataaaggaaatttattttcattgcaatagtgtgttggaattttttgtgtctctcacactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctagaaagcttcccggggggatctg

SEQ ID NO: 18 - DNA, 5051 bp, synthetic construct
  source 1..5051: mol_type = other DNA
ggccactccctctctgcgcgctcgctcgctcactgaggccgggcgaccaaaggtcgcccggcctcagtgagcgagcgagcgcgcagagagggagtggccaactccatcactaggggttcctggaggggtggagtcgtgacctaggggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtcagatcagatctttgtcgatcctaccatccactcgacacacccgccagcggccgcgttggtatcaaggttacaagacaggtttaaggagaccaatagaaactgggcatgtggagacagagaagactcttgggtttctgataggcactgactctcttcctttgtcctgttcccatttcagaagcttccgagctctcgaattaaataagagagaaaagaagagtaagaagaaatataagagccaccatggccgtgtgtggactgggcagcagactgggactgggcagcagactgggactgagaggatgctttggcgccgctagactgctgtaccctagattccagtctagaggccctcaaggcgtggaggatggagacagaccccagcccagctccaaaacccctagaatccccaagatctacaccaagaccggcgacaagggcttcagcagcaccttcaccggcgagagaagacccaaggatgaccaagtgttcgaggccgtgggcaccaccgacgaactgtccagcgccattggcttcgctctggagctggtgaccgagaagggacacacctttgccgaggagctgcagaagattcagtgcacactgcaagacgtgggcagcgctctggccaccccttgcagctccgctagggaagcccatctgaagtacaccaccttcaaggccggccctattctggagctcgagcagtggatcgacaaatacaccagccagctgcctcctctgaccgccttcattctgccttccggaggcaagatctccagcgctctgcacttctgtagagccgtgtgtagaagagccgaaagaagagtggtccctctggtgcagatgggagagaccgacgccaacgtggccaagtttctgaatagactgagcgattatctgttcacactggctagatacgccgccatgaaggagggcaaccaagagaagatctacatgaagaacgacccctccgccgagagcgaaggactgtgaactcgagataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatctccgatctttttccctctgccaaaaattatggggacatcatgaagccccttgagcatctgacttctggctaataaaggaaatttattttcattgcaatagtgtgttggaattttttgtgtctctcacactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctcgagatctagaaagcttcccggggggatctg

SEQ ID NO: 19 - DNA, 6141 bp, synthetic construct
  source 1..6141: mol_type = other DNA
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggcttctagactgcgcgctcgctcgctcactgaggccgcccgggcaaagcccgggcgtcgggcgacctttggtcgcccggcctcagtgagcgagcgagcgcgcagagagggagtggccaactccatcactaggggttcctttaattaaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtactgaattcaaataagagagaaaagaagagtaagaagaaatataagagccaccatggcggtctgcggattgggttcacgattgggccttgggtctaggctcggcctgcgaggttgtttcggagctgctagattgttgtacccacgattccaaagcaggggaccacaaggtgtggaagacggtgacaggcctcagccctcaagcaaaacccctcggattccgaaaatatatacaaagacaggagacaaaggcttttcaagcacgtttactggcgaacgaaggcctaaagacgatcaagtttttgaggcggtagggacaactgacgaactttcatctgcgataggcttcgcgctcgagttggtgacggagaagggtcatacattcgctgaagaacttcagaaaattcagtgtacgctccaagatgttggcagtgcgcttgcgacaccttgcagctcagcaagggaagcgcatttgaagtacaccactttcaaagcgggcccgattctcgaacttgaacaatggattgacaaatatacaagccaactgccgcctcttaccgccttcatcctccctagtggtggcaaaatttcttccgcgcttcatttttgtagggccgtgtgtcgccgcgccgagagacgggtggttccgttggtacagatgggtgaaacagatgcaaacgtggcaaagttccttaaccgcctcagcgactacttgttcactctcgctaggtatgccgccatgaaagaagggaatcaagagaagatctatatgaagaatgacccgagcgctgaaagcgagggactttgaggcctcgagataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatcgatatcgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagactagtcctgcaggtacctaggaacccctagtgatggagttggccactccctctctgcgcgctcgctcgctcactgaggccgggcgaccaaaggtcgcccgacgcccgggctttgcccgggcggcctcagtgagcgagcgagcgcgcagcatatgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 20 - DNA, 5140 bp, synthetic construct
  source 1..5140: mol_type = other DNA
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggcttctagactgcgcgctcgctcgctcactgaggccgcccgggcaaagcccgggcgtcgggcgacctttggtcgcccggcctcagtgagcgagcgagcgcgcagagagggagtggccaactccatcactaggggttcctttaattaaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtcagatcagatctttgtcgatcctaccatccactcgacacacccgccagcggccgcgttggtatcaaggttacaagacaggtttaaggagaccaatagaaactgggcatgtggagacagagaagactcttgggtttctgataggcactgactctcttcctttgtcctgttcccatttcagaagcttccgagctctcgaattaaataagagagaaaagaagagtaagaagaaatataagagccaccatggccgtgtgtggactgggcagcagactgggactgggatctagactgggactgagaggctgcttcggcgctgctagactgctgtaccctagattccagtccagaggaccccaaggcgtcgaggatggagatagaccccagccctcctccaagacccctagaatccccaagatctacaccaagaccggcgacaagggcttcagctccacctttaccggcgagaggagacccaaggacgatcaagtgttcgaggccgtgggaaccaccgacgagctgagctccgctatcggcttcgctctggagctggtgacagagaagggccacacctttgccgaggagctgcagaagatccagtgcacactgcaagacgtgggcagcgctctggctacaccttgcagcagcgctagagaggcccatctgaaatacaccaccttcaaagccggccctattctggagctggaacagtggatcgacaagtacaccagccagctgcctcctctgacagccttcattctgcccagcggcggaaagatctccagcgctctgcacttctgcagagccgtctgcagaagggctgagagaagggtggtgcctctggtgcagatgggcgagaccgatgccaacgtggccaagttcctcaatagactgtccgattatctgttcacactggctagatatgccgccatgaaggagggcaaccaagagaagatctacatgaagaacgatcccagcgccgagagcgagggcctctgaactcgagataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatctccgatctttttccctctgccaaaaattatggggacatcatgaagccccttgagcatctgacttctggctaataaaggaaatttattttcattgcaatagtgtgttggaattttttgtgtctctcacactagtccactccctctctgcgcgctcgctcgctcactgaggccgggcgaccaaaggtcgcccgacgcccgggctttgcccgggcggcctcagtgagcgagcgagcgcgcagagagggacagatccgggcccgcatgcgtcgacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatgacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 21 - protein, 250 aa, Homo sapiens
MAVCGLGSRLGLGSRLGLRGCFGAARLLYPRFQSRGPQGVEDGDRPQPSSKTPRIPKIYTKTGDKGFSSTFTGERRPKDDQVFEAVGTTDELSSAIGFALELVTEKGHTFAEELQKIQCTLQDVGSALATPCSSAREAHLKYTTFKAGPILELEQWIDKYTSQLPPLTAFILPSGGKISSALHFCRAVCRRAERRVVPLVQMGETDANVAKFLNRLSDYLFTLARYAAMKEGNQEKIYMKNDPSAESEGL

SEQ ID NO: 22 - protein, 32 aa, Homo sapiens
MAVCGLGSRLGLGSRLGLRGCFGAARLLYPRF

SEQ ID NO: 23 - protein, 218 aa, Homo sapiens
QSRGPQGVEDGDRPQPSSKTPRIPKIYTKTGDKGFSSTFTGERRPKDDQVFEAVGTTDELSSAIGFALELVTEKGHTFAEELQKIQCTLQDVGSALATPCSSAREAHLKYTTFKAGPILELEQWIDKYTSQLPPLTAFILPSGGKISSALHFCRAVCRRAERRVVPLVQMGETDANVAKFLNRLSDYLFTLARYAAMKEGNQEKIYMKNDPSAESEGL